SEQUENCE LISTING (16 sequences)

SEQ ID NO: 1 - DNA, 20 bp, Artificial sequence
  Single strand DNA oligonucleotide
tgttcacaggcttgactcca

SEQ ID NO: 2 - DNA, 24 bp, Artificial sequence
  Single strand DNA oligonucleotide
caaactgtataggtgaatggctct

SEQ ID NO: 3 - DNA, 21 bp, Artificial sequence
  Single strand DNA oligonucleotide
gactgggcatgaccctaagtt

SEQ ID NO: 4 - DNA, 20 bp, Artificial sequence
  Single strand DNA oligonucleotide
cgctcctctccagatgggta

SEQ ID NO: 5 - DNA, 22 bp, Artificial sequence
  Single strand DNA oligonucleotide
aatttgccatttaccgatggga

SEQ ID NO: 6 - DNA, 21 bp, Artificial sequence
  Single strand DNA oligonucleotide
agcatccaacaccataggtcc

SEQ ID NO: 7 - DNA, 20 bp, Artificial sequence
  Single strand DNA oligonucleotide
accctaatctagtcccgtcc

SEQ ID NO: 8 - DNA, 20 bp, Artificial sequence
  Single strand DNA oligonucleotide
cagccaaaaccagatgacag

SEQ ID NO: 9 - DNA, 20 bp, Artificial sequence
  Single strand DNA oligonucleotide
cattggtgatggtattgcgc

SEQ ID NO: 10 - DNA, 19 bp, Artificial sequence
  Single strand DNA oligonucleotide
tcccaaacacgacaactcc

SEQ ID NO: 11 - protein, 303 aa, Homo sapiens
MADAASQVLLGSGLTILSQPLMYVKVLIQVGYEPLPPTIGRNIFGRQVCQLPGLFSYAQHIASIDGRRGLFTGLTPRLCSGVLGTVVHGKVLQHYQESDKGEELGPGNVQKEVSSSFDHVIKETTREMIARSAATLITHPFHVITLRSMVQFIGRESKYCGLCDSIITIYREEGILGFFAGLVPRLLGDILSLWLCNSLAYLVNTYALDSGVSTMNEMKSYSQAVTGFFASMLTYPFVLVSNLMAVNNCGLAGGCPPYSPIYTSWIDCWCMLQKEGNMSRGNSLFFRKVPFGKTYCCDLKMLI

SEQ ID NO: 12 - protein, 195 aa, Mus musculus
MDSEVSNGSGLGAKHITDLLVFGFLQSSGCTRQELEVLGRELPVQAYWEADLEDELQTDGSQASRSFNQGRIEPDSESQEEIIHNIARHLAQIGDEMDHNIQPTLVRQLAAQFMNGSLSEEDKRNCLAKALDEVKTAFPRDMENDKAMLIMTMLLAKKVASHAPSLLRDVFHTTVNFINQNLFSYVRNLVRNEMD

SEQ ID NO: 13 - protein, 195 aa, Homo sapiens
MDCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 14 - DNA, 19 bp, Artificial Sequence
  SiRNA sequence that corresponds with human Mtch2
ctcgagagatgatcgctcg

SEQ ID NO: 15 - DNA, 21 bp, Artificial Sequence
  oligonucleotides against MTCH2
tcctcacccttgtcactctcc

SEQ ID NO: 16 - DNA, 2524 bp, Homo sapiens
tcgcttggtggctccgtctgtctgtccgtccgcccgcgggtgccatcatggcggacgcggccagtcaggtgctcctgggctccggtctcaccatcctgtcccagccgctcatgtacgtgaaagtgctcatccaggtgggatatgagcctcttcctccaacaataggacgaaatatttttgggcggcaagtgtgtcagcttcctggtctctttagttatgctcagcacattgccagtatcgatgggaggcgcgggttgttcacaggcttaactccaagactgtgttcgggagtccttggaactgtggtccatggtaaagttttacagcattaccaggagagtgacaagggtgaggagttaggacctggaaatgtacagaaagaagtctcatcttcctttgaccacgttatcaaggagacaactcgagagatgatcgctcgttctgctgctaccctcatcacacatcccttccatgtgatcactctgagatctatggtacagttcattggcagagaatccaagtactgtggactttgtgattccataataaccatctatcgggaagagggcattctaggatttttcgcgggtcttgttcctcgccttctaggtgacatcctttctttgtggctgtgtaactcactggcctacctcgtcaatacctatgcactggacagtggggtttctaccatgaatgaaatgaagagttattctcaagctgtcacaggattttttgcgagtatgttgacctatccctttgtgcttgtctccaatcttatggctgtcaacaactgtggtcttgctggtggatgccctccttactccccaatatatacgtcttggatagactgttggtgcatgctacaaaaagaggggaatatgagccgaggaaatagcttatttttccggaaggtcccctttgggaagacttattgttgtgacctgaaaatgttaatttgaagatgtggggcagggacagtgacatttctgtagtcccagatgcacagaattatgggagagaatgttgatttctatacagtgtggcgcgcttttttaataatcatttaatcttgggaaaattcaggtgtttggtgtctgccttttttgttcttttttccagcacaacataacttaccactgatactccccctttagttattctgaattaggatatttttgctccaaattcttattttacttaaccagaagggaaaaaaagttgtattttcctgaagctacaggcactttgtcatgtgatttttgagtctcaatttaaggctttgtaaaatgaagagtagaattccaagaaaaatgagaaataattttgtaaaacttaacaaaatcactaaattaaactatatgggaggttatgaattactttttcttgggtagaccctaaaatgtcagtagcatgcaccagaatctgactcccattatgcttctaagcacatttcattgaccttgtctctcatacttcaagaaaaggacagtacattgctacattaccctagaaagtctgtgtgaggatctgccccttcagtctgttattgcaaagtaataaaatgtcacctacagggagcctctgagcctactctagttcaagaggctacctgaaaaaaaataaataagataaagggtcagcaacaacaaagaaaaagacaattacagaaaataagcaagatttggaaaggaagtataatggcacttttttcctcaaaggaagttcttgttttcctcaaaggaagttcttgttttcacataaaatatgaaaagcagatcctgcaggagtaacccccttctttaagagccaagtatttgccagtgcttaaattacaccataccgttctaattatatataatcttttgttcttcagttttttgttttgtttcctttttgttattgttgccgaaggtgagtagttttgcatttctgatgacagccttggaaagtatatttgtaactccatgtctggtaatgccaacccaagtcgacatgggtcttaggacactgaccacctcacatgccataccctcagttaagcatgttaacatttataggaggaaaaaaatcactttgggagaaaataaaattcaactcaagcataaagcttctgtttactcaggccttctaaaaagcaggttaaaatgctctaaaatgagaaagcctgtggtttcacttatttatataactcactgggacattgccaaatgagtaagcacttaattcgctgcttctgagacttctctgtcaaaacagccccactgataatattagacagaacgagaatgcaggggtctcttccctcccctggggtttaggaagctcatgaggagctcggcttaaaatgtctttgatgtctcttcctttgtctcaaaaagtaatgtcaattttatatactatttcaatattactatctgcatttgttttaatataaaaatgtttgctgcctacctttttctcccaaaaaatctttaagtaaagatgatctgggaaaatgtaaaaaaaaaaaaaaaaataaaaaaaa

(19) United States
(12) Patent Application Publication
(10) Pub. No.: US 2018/0042994 A1
(43) Pub. Date: Feb. 15, 2018

(54) METHODS OF TREATING DISEASES RELATED TO MITOCHONDRIAL FUNCTION

(71) Applicant: Yeda Research and Development Co. Ltd., Rehovot (IL)

(72) Inventors: Atan GROSS, Nes Ziona (IL); Yehudit ZALTSMAN, Rehovot (IL); Liat PINKAS, Rehovot (IL); Maria MARYANOVICH, Rehovot (IL); Liat BUZAGLO, Rehovot (IL); Antonella RUGGIERO, Rehovot (IL); Amir BAHAT, Rehovot (IL); Andres GOLDMAN, Rehovot (IL); Michael MULLOKANDOV, Rehovot (IL)

(21) Appl. No.: 15/525,618

(22) PCT Filed: Nov. 17, 2015
(86) PCT No.: PCT/IL2015/051108

§ 371 Date: May 10, 2017

Related U.S. Application Data
(60) Provisional application No. 62/080,518, filed on Nov. 17, 2014.

(51) Int. Cl. A61K 38/17; C12N 5/00

(52) U.S. Cl. CPC A61K 38/1761; C12N 5/0018

(57) ABSTRACT

Uses of agent which downregulate an activity or expression of a component participating in the mitochondrial apoptotic pathway are disclosed.

### FIELD AND BACKGROUND OF THE INVENTION

The present invention, in some embodiments thereof, relates to methods of treating diseases related to mitochondrial function.

Programmed cell death or apoptosis is critical for both the development and maintenance of tissues. Caspases, a family of cysteine proteases, are the major executioners of the apoptotic process, whereas the Bcl-2 family members are the major regulators of this process. The Bcl-2 family is composed of both pro-apoptotic and anti-apoptotic proteins. A subset of the pro-apoptotic factors are the BH3-only proteins, which act as sentinels of internal damage.

The BH3-only BID protein is a critical activator of the mitochondrial apoptotic program. Activation of the tumor necrosis factor (TNF)/Fas death receptors leads to caspase-8-mediated cleavage of BID to the truncated form, tBID, that translocates to the mitochondria to activate Bax and Bak, resulting in mitochondrial outer membrane permeabilization (MOMP). The requirement for BID in the Fas death pathway was demonstrated in BID-deficient mice, which are resistant to Fas-induced hepatocellular apoptosis; the requirement for Bax and Bak was demonstrated in Bax/Bak double-knockout cells, which are resistant to multiple apoptotic stimuli as well as to tBID and several other BH3-only molecules.

It was previously shown that in TNF-α-activated haematopoietic FL5.12 cells, tBID becomes part of a roughly 45 kDa crosslinkable mitochondrial complex that comprises mitochondrial carrier homologue 2 (MTCH2 (Grinberg, M. et al. Mol. Cell. Biol. 25, 4579-4590 (2005); Gross, A. J. Bioenerg. Biomembr. 37, 113-119 (2005)); also named Met-induced mitochondrial protein (MIMP)). MTCH2/MIMP is a 33 kDa protein related to the mitochondrial carrier (MC) protein family. It was further shown that that MTCH2/MIMP is an outer mitochondrial membrane protein with a critical function in the mitochondrial outer membrane permeabilization process by facilitating the recruitment of tBID to mitochondria.

Background art includes Zaltsman et al., Nature Cell Biology, Volume 12, No. 6, 2010, pages 553-562; Bauer et al., Am J Clin Nutr 2009; 90:951-9: WO 2010/116375 and U.S. Patent Application No. 20090124564.

### SUMMARY OF THE INVENTION

According to an aspect of some embodiments of the present invention there is provided a method of increasing mobilization of hematopoietic precursors from the bone marrow to the peripheral blood in a subject in need thereof, the method comprising:
(a) administering to the subject an agent which downregulates an activity and/or expression of a component participating in the mitochondrial apoptotic pathway; and
(b) harvesting the hematopoietic precursors from the peripheral blood.

According to an aspect of some embodiments of the present invention there is provided an agent which downregulates an activity and/or expression of a component participating in the mitochondrial apoptotic pathway thereof for use in the manufacture of a medicament for increasing mobilization of hematopoietic precursors in a subject.

According to an aspect of some embodiments of the present invention there is provided a kit comprising an agent which downregulates an activity and/or expression of a component participating in the mitochondrial apoptotic pathway and a mobilization factor selected from the group consisting of a growth factor, a cytokine, a chemokine and a polysaccharide.

According to an aspect of some embodiments of the present invention there is provided a method of propagating stem cells comprising culturing the stem cells in a culture medium comprising an agent that upregulates an activity and/or expression of a component participating in the mitochondrial apoptotic pathway under conditions that allow propagation of the stem cells, but do not allow differentiation of the stem cells, thereby propagating the stem cells.

According to an aspect of some embodiments of the present invention there is provided a culture medium comprising an agent that upregulates an activity and/or expression of a component participating in the mitochondrial apoptotic pathway.

According to an aspect of some embodiments of the present invention there is provided a method of treating a mitochondrial related disease or condition in a subject in need thereof comprising administering to the subject a therapeutically effective amount of an agent which regulates an amount and/or activity of mitochondrial carrier homologue 2 (MTCH2) or an agonist thereof, thereby treating the mitochondrial related disease or condition.

According to an aspect of some embodiments of the present invention there is provided an agent which regulates an amount and/or activity of MTCH2 or an agonist thereof, for use in treating a mitochondrial related disease or condition.

According to some embodiments of the invention, the subject is a human subject.

According to some embodiments of the invention, the subject is a donor subject.

According to some embodiments of the invention, the subject is a recipient subject.

According to some embodiments of the invention, the recipient subject is in need of an organ transplantation.

According to some embodiments of the invention, the organ transplantation comprises stem cell transplantation.

According to some embodiments of the invention, the stem cell transplantation is allogeneic with respect to the recipient.

According to some embodiments of the invention, the stem cell transplantation is autologous with respect to the recipient.

According to some embodiments of the invention, the subject has an immune deficiency.

According to some embodiments of the invention, the immune deficiency comprises a hematologic disorder or condition.

According to some embodiments of the invention, the hematologic disorder or condition is a hematologic cancer.

According to some embodiments of the invention, the method further comprises administering a mobilization factor selected from the group consisting of a growth factor, a cytokine, a chemokine and a polysaccharide.

According to some embodiments of the invention, the mobilization factor is G-CSF.

According to some embodiments of the invention, the at least one component participating in the mitochondrial-related apoptotic pathway is selected from the group consisting of mitochondrial carrier homologue 2 (MTCH2), BID, Caspase-8, Bax and Bak.

According to some embodiments of the invention, the at least one component participating in the mitochondrial-related apoptotic pathway is MTCH2.

According to some embodiments of the invention, the culture medium further comprises stem cells.

According to some embodiments of the invention, the stem cells comprise pluripotent stem cells.

According to some embodiments of the invention, the pluripotent stem cells comprise embryonic stem cells.

According to some embodiments of the invention, the stem cells comprise hemapoietic stem cells.

According to some embodiments of the invention, the at least one component participating in the mitochondrial-related apoptotic pathway is selected from the group consisting of mitochondrial carrier homologue 2 (MTCH2), BID, Caspase-8, Bax and Bak.

According to some embodiments of the invention, the at least one component participating in the mitochondrial-related apoptotic pathway is MTCH2.

According to some embodiments of the invention, the pluripotent stem cells retain the ability to differentiate into each of the three germ layers.

According to some embodiments of the invention, the culture medium further comprises serum or serum replacement.

According to some embodiments of the invention, the agonist comprises BH3-interacting domain death agonist (BID).

According to some embodiments of the invention, the regulates is down-regulates.

According to some embodiments of the invention, the disease or disorder is radiation injury.

According to some embodiments of the invention, the disease is a fat-related disease.

According to some embodiments of the invention, the condition is reduced exercise endurance.

According to some embodiments of the invention, the fat-related disease is selected from the group consisting of diabetes, metabolic syndrome and obesity.

According to some embodiments of the invention, the disease is a cardiac disease.

According to some embodiments of the invention, the agent is an oligonucleotide directed to an endogenous nucleic acid sequence expressing the MTCH2 or the agonist thereof.

According to some embodiments of the invention, the regulates is up-regulates.

According to some embodiments of the invention, the disease or disorder is selected from the group consisting of retarded growth, hematological cancer, hyperactivity, learning and memory disability and male infertility.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G: MTCH2^{F/F} Vavl-Cre⁺ HSCs are less quiescent and demonstrate a competitive disadvantage in bone marrow repopulation.

(1A) MTCH2-deletion driven by Vav1-Cre results in haematopoietic-specific MTCH2 knockout. Relative MTCH2 gene expression was evaluated in freshly isolated cells from the indicated tissues by quantitative real-time PCR and normalized to the hypoxanthine phosphoribosyltransferase (HPRT) housekeeping gene. Results are presented as mean relative quantity±s.d. (*p≦0.05, n=4 mice).

(1B) Reduced HSC frequency and enhanced lymphoid biased differentiation upon MTCH2 deletion. Mice were sacrificed and their total bone marrow cells were evaluated for the size of HSCs (CD150⁺CD48⁻CD41⁻lin⁻Sca-1⁺c-kit⁺), multipotent progenitors (MPPs; CD150⁻CD48⁻CD41⁻lin⁻Sca-1⁺c-kit⁺), common lymphoid progenitor (CLPs; IL7R⁺lin⁻Sca-1⁻c-kit⁺) and common myeloid progenitor (CMPs; FCγR⁻lin⁻Sca-1⁻c-kit⁺) populations. Results are presented as mean percent±s.d. (**p<0.005, n=5 mice).

(1C) MTCH2^{F/F} Vav1-Cre⁺ HSCs are less quiescent. The portion of quiescent HSCs (Ki-67⁻DAPI^{low}lin⁻Sca-1⁺c-kit⁺) was evaluated by staining of freshly isolated bone marrow cells. Results are presented as means±s.d. (*p<0.05, n=4 mice).

(1D) Enhanced proliferation in MTCH2-deficient HSCs and committed progenitors. Mice were administered BrdU by three daily i.p. injections and 2 hours following the last administration the levels of BrdU incorporation in HSCs, LSKs (lin⁻Sca- 1⁺c-kit⁺), and restricted progenitors (lin⁻c-kit⁺) were evaluated as described in the Methods. Results are presented as means±s.d. (*p≦0.05; **p≦0.005, n=4 mice).

(1E) MTCH2-deficient bone marrow cells show reduced colony formation ability. Mice were sacrificed and 100,000 bone marrow mono-nuclei cells (MNCs) were plated in methyl-cellulose media supplemented with selected cytokines. Results are presented as means±s.d. (*p≦0.05, n=3 mice).

(1F) MTCH2-deficient cells show competitive disadvantage in bone marrow repopulation. Total bone marrow cells were transplanted at a 1:1 ratio into lethally irradiated hosts as described in the Methods. The peripheral blood engraftment (left panel) and bone marrow engraftment 20 weeks following transplantation (right panel) results are presented as means±s.d. of donor derived cells (**p<0.005, n=5 mice).

(1G) MTCH2-deficient HSPCs show increased mobilization to peripheral blood. Peripheral blood of mice was evaluated for the presence of LSK HSPCs and lin⁻c-kit⁺ restricted progenitors. Results are presented as means±s.d. (**p<0.005, n=8 mice).

FIGS. 2A-F: Loss of MTCH2 primes mitochondrial oxidative metabolism.

(2A) Increased mitochondrial respiration in MTCH2^{F/F} Vavl-Cre⁺ HSPCs. Freshly isolated bone marrow cells from untreated mice were enriched for c-kit⁺ cells by magnetic cell sorting. Left panel: Oxygen consumption rates (OCR) were measured using the Seahorse XF24 analyzer under basal conditions and in response to 0.5 μM oligomycin (complex V inhibitor), 0.5 μM fluoro-carbonyl cyanide phenylhydrazone (FCCP; Uncoupler) or 1 μM Antimycin A and Rotenone (Complex III and I inhibitors, respectively). Middle panel (average of results shown in left panel): Basel OCR and Maximum OCR (as indicated by OCR after injection of oligomycin and subsequent FCCP). Right panel (average of results shown in left panel): Spare Respiratory Capacity (SRC), indicated by maximum OCR calculated as percentage of baseline OCR. A representative tracing of a typical experiment of four is shown. The data represent mean±s.d. (*p≦0.05, n=8 mice).

(2B) Reduced mitochondrial NADH levels in MTCH2-deficient HSPCs. Freshly isolated total bone marrow cells were labeled for HSPC markers (lin⁻Sca-1⁺c-kit⁺) and NADH autofluorescence was measured by flow cytometry. NADH redox index was calculated as described in the Methods. Results are represented as mean percent±s.d. (**p≦0.005, n=4 mice).

(2C) Increased gene expression of nuclear-encoded subunits of the mitochondrial respiratory complexes in MTCH2-deficient HSPCs. Freshly isolated bone marrow cells from untreated mice were enriched for c-kit⁺ cells by magnetic cell sorting and relative gene expression was evaluated by quantitative real-time PCR and normalized to HPRT expression. Results are presented as mean relative quantity±s.d. (*p≦0.05, n=4 mice).

(2D) Elevated mitochondrial membrane potential in MTCH2^{F/F} Vavl-Cre⁺ HSCs. Freshly isolated total bone marrow cells were labeled for HSC markers (CD150⁺CD48⁻CD41⁻lin⁻Sca-1⁺c-kit⁺) and stained with Tetramethylrhodamine (TMRE) and Mito Tracker Green (MTG), to evaluate mitochondrial membrane potential relative to total mitochondrial mass, respectively. Results are presented as ratio of TMRE to MTG fluorescence±s.d. (*p<0.05, n=4 mice).

(2E) Moderate increase in ROS levels in MTCH2-deficient HSCs. Freshly isolated total bone marrow cells were labeled for HSC markers (CD150⁺CD48⁻CD41⁻lin⁻Sca- 1⁺c-kit⁺) and stained with either hydroethidine (DHE) to assess intracellular ROS levels or MitoSOX to assess mitochondrial ROS levels. Results are presented as fold change in ROS levels relative to MTCH2^{FF} cells±s.d. (*p<0.05, n=4 mice).

(2F) MTCH2-deficient HSPC mitochondria are significantly enlarged. Freshly isolated bone marrow cells from untreated mice were enriched for c-kit⁺ cells by magnetic cell sorting and prepared for electron microscopy (EM) analysis as described in the Methods. Left panel: Representative EM images are presented (4800× and 23000× magnification and scale bars 1 μm and 200 nm for the entire cell and enlarged mitochondria images, respectively). Middle and Right panels: Analysis of mitochondria area relative to cell area and number of mitochondria per cell.

FIGS. 3A-J: MTCH2^{F/F} Vav1-Cre⁺ HSPCs possess higher levels of ATP and are resistant to irradiation-induced apoptosis in vivo.

(3A) MTCH2-deficient HSPCs are resistant to TBI-induced death. Mice were either left untreated or subjected to 6.5 Gy TBI. Three hours post exposure, mice were sacrificed and the amount of LSK cells in the bone marrow was quantified. Results are presented as means±s.d. (**p≦0.005, n=6 mice).

(3B) MTCH2-deficient bone marrow cells exhibit unperturbed colony formation in culture following irradiation. Freshly isolated total bone marrow MNCs were excised and 100,000 cells were plated in methylcellulose culture supplemented with cytokines. Cultures were then either left untreated or exposed to 2.5 Gy ionizing radiation and left to grow for seven days. Results are presented as percent survival relative to non-treated cultures of each genotype±s.d. (*p<0.05, n=3 mice).

(3C) MTCH2-deficient HSPCs are resistant to TBI-induced caspase-3 activation. Mice were either left untreated or subjected to 6.5 Gy TBI. Half an hour post exposure, mice were sacrificed and the amount of caspase-3 activity in c-kit⁺ enriched cells was quantified. Results are presented as means±s.d. (**p<0.005, n=3 mice).

(3D) MTCH2-deficient HSPCs are resistant to TBI-induced caspase-3 cleavage. Mice were treated as in (3C), the c-kit⁺ enriched cells were lysed, separated by SDS-PAGE and analyzed by Western blot using an anti-cleaved caspase-3 antibody (top panel). Actin is used as an internal standard (lower panel). Results are presented for six mice from each genotype.

(3E) MTCH2-deficient HSPCs possess higher levels of ATP. Mice were either left untreated or subjected to 6.5 Gy TBI. Half an hour post exposure, mice were sacrificed and the amount of ATP in c-kit⁺ enriched cells was quantified as described in the Methods. Results are presented as means±s.d. (**p<0.005, n=3 mice).

(3F) Schematic representation of the MTCH2 pathway repressing mitochondria OxMetab to regulate haematopoietic stem cell fate. Inhibition of MTCH2 leads to an increase in mitochondrial OXPHOS, which results in an increase in ATP production and in ROS levels. ROS supplies the signal for the rapid transition from stem to progenitor cell and the increase in ATP sustains stem/progenitor cell viability.

(3G-I) FACS analysis for GFP positive cells; (G) Blood test of mouse transplanted with MTCH2 knockout pre-leukemic cells, (H) Bone marrow and (I) Spleen of mouse transplanted with MTCH2 knockout pre-leukemic cells (two month post-transplantation).

(3J) Graph illustrating the survival proportion for mice transplanted with MTCH2^{F/F} (green) and MTCH2^{F/F} Vavl-cre+(red) derived pre-leukemic cells.

FIG. 4 illustrates representative blot of lysates of various tissues of the MKO and WT mice. The Western blot analysis using anti-MTCH2 antibodies demonstrates a specific absence of MTCH2 from the skeletal muscle and heart in the MKO mice.

FIGS. 5A-B illustrate that MKO mice eat and drink more. (A) Metabolic cage study of 12-14 weeks old male MKO and WT mice (n=16 for each genotype). Average 72 h basal food intake. (B). Average 72 h water intake. Data are presented as mean±SEM. *P<0.05.

FIGS. 6A-B illustrate that MKO mice are less sensitive to HFD feeding. (A) Percentage of weight gain of MKO mice and their WT littermates on NCD, LFD (10% fat) and HFD (45% fat) for 13 weeks. Two way ANOVA shows significance of *P<0.05. (B) Mice at the end of the 13 weeks HFD were analyzed for their % of lean and fat using the EchoMRI device. On HFD, MKO mice have less % fat mass. Data are the means±SEM.

FIGS. 7A-D illustrate that knockout of MTCH2 in Myofibers increases Mitochondrial respiration and glucose uptake. (A) Myotubes were examined for the basal and maximal Oxygen Respiration Rate (OCR) using XF Analysis instrument. Maximal respiration was induced using 4 μM FCCP. Both basal and maximal respiration of the KO myofibers were elevated versus WT myofibers. This effect was observed in glucose metabolism using 25mM D-Glucose (B), and in Fatty acid metabolism using 200 mM Palmitate (C). Data are presented as mean±STD of four independent experiments. **, P<0.01. (D) Glucose uptake was measured using 50 μM of 2-NBDG. Muscle fibers were subjected overnight in low glucose (5 mM). 2-NBDG was added for 30 minute and after FACS devise was used for quantification of glucose uptake (n=9).

FIGS. 8A-B illustrate that knocking out MTCH2 leads to a significant increase in mitochondrial size in gastrocnemius skeletal muscle fibers in vivo. (A) Representative EM micrographs (2900× magnification) of skeletal muscle from WT and MKO mice. (B) Average area of mitochondria measured from digitized images (n=3 per genotype). Data are presented as mean±SEM. **, P<0.01.

FIGS. 9A-C illustrate that MKO mice are better at endurance exercise than WT mice. (A) Diagram of the endurance profile. (B) WT and MKO mice underwent exhaustion protocol on the treadmill. MKO demonstrated better endurance since the distance achieved prior the exhaustion was higher (n=15 for each genotype). (C) Metabolic cages were used to measure 02 consumption during the mice exercise applying the exhaustion profile. Two way ANOVA test revealed significantly that MKO consume more O₂ during the exercise (n=16 for each genotype). Data are presented as mean±SEM. *, P<0.05.

FIGS. 9D-E illustrate that MTCH2^{FIF} MCK-Cre⁺ mice show an increase in energy expenditure. (9D) Heat production in absolute values (kcal/24 h) controlled for lean body weight as a covariant. (9E) MTCH2^{F/F} MCK-Cre⁺ mice demonstrate increase in heat production. ANCOVA was performed using univariate general linear model module in SPSS. (*p<0.05, n=8 mice).

FIGS. 9F-K illustrate that MTCH2^{FIF} MCK-Cre⁺ mice show increase in heart function. Echocardiographic analysis of control and MTCH2^{F/F} MCK-Cre⁺ mice show similar heart size measurements: (9F) diameter (9G) systole and (9H) diastole left ventricular anterior wall (LVAW) and left ventricular posterior wall (LVPW). Simultaneously, MTCH2^{F/F} MCK-Cre⁺ mice show increase in heart function measurements: (9I) ejection fraction (EF) (9J) stroke volume and (9K) fractional shortening (FS). The data represent mean ±SEM (*p≦0.05, n=5 mice).

FIG. 10 illustrates specific depletion of MTCH2 in the muscle of MyKO mice. A blot of lysates of various tissues of the MyKO and WT mice. The Western blot analysis using anti-MTCH2 antibodies demonstrates the absence of MTCH2 from the skeletal muscle in MyKO mice, while an increased amount of MTCH2 is observed in the liver, heart and the brain tissues (n=1).

FIGS. 11A-E illustrate that MyKO mice are smaller than WT mice upon birth. (A) A representative picture of two 4 day-old MyKO mice (on the left) and one WT mouse (on the right). (B) Weight of females mice before weaning. Females MyKO mice are significantly smaller (n=15 for each genotype). (C) Weight of females MyKO and WT mice during 8 week. Two ANOVA test shows that females MyKO mice have significantly less weight (n=15 for each genotype). (D) Weight of male mice before weaning. Males MyKO mice are significantly smaller (n=15 for each genotype). (E) Weight of males MyKO and WT mice during 8 week. Two ANOVA test shows that males MyKO mice have significantly less weight (n=15 for each genotype). Data are presented as mean±SEM. **, P<0.01.*, P<0.05.

FIGS. 12A-B illustrate Cre expression pattern and MTCH2 depletion in adult brain. (A) Sagittal floating section of adult Rosa26-CamKIIa cre mouse brain was stained for β-galactosidase activity. (B) Western blot analysis on purified mitochondria extracted from the different brain regions of WT and BKO (brain knock-out).

FIG. 13 illustrates that BKO female gain 5 to 10% less weight compared to the WT littermates. Data are presented as mean±s.e.m. t-test: *=p<0,05. (n=30mice/genotype).

FIGS. 14A-D illustrate that BKO female are hyperactive during night cycle, produce more heat and eat more, especially after o.n. starvation. Females matched for body weight were monitored using TSE Labmaster/ Phenomaster system. (A) Oxygen consumption; (B) Food intake in steady state and after an overnight starvation; (C) Heat production; (D) Home cage locomotor activity. Data are presented as mean±s.e.m. t-test: *=p<0,05.

FIGS. 15A-B illustrate hormone levels in BKO mice. (A,B) Insulin and leptin levels were measured by ELISA kit on serum samples. Interestingly, no differences in insulin levels were observed.

FIGS. 16A-F illustrate behavioral characterization of BKO females. (A) Daily wheel running activity in females was monitor using the running wheel system. BKO females cover significantly more distance compare to WT littermates. T-test: **=p<0,01 (B) Locomotor activity was measured in an open field arena for 40 minutes in low illumination condition (n=10 mice/genotype). (C) Latency to fall from an accelerated rotarod in 5 consecutive trials. BKO female performed worst compare to the control (n=23mice/genotype). (D,E) Morris Water Maze results show that BKO females have impaired spatial memory, learning and cognitive mapping (n=12 mice/genotype). Data are presented as mean±s.e.m. Statistic: Two-Way Anova-Repeated Measurements *=p<0.05; **=p<0.01. (F) Locomotor activity was monitored in 10 week old females (4 animals/genotype) in the TSE Labmaster/ Phenomaster system. BKO females were more active than the wild-type littermates (MTCH2^{F/F} vs BKO injected with saline; p=0.034; 2way ANOVA- Multiple comparison). The same mice receive 2 mg/kg intraperitoneal injection of ritalin (Novartis), notably the BKO treated with ritalin behave like the control group (BKO saline vs BKO 2 mg/Kg Ritalin; p=0.03; 2way ANOVA- Multiple comparison).

FIGS. 17A-B illustrate that BKO males are less fertile compared to the WT littermates and their spermatozoa motility is dramatically reduced. (A) 8 week-old MTCH2^{f/f} (n=1) and BKO (n=5) males were mated with 2 C57BL6J females for a period of 4 months. The total number of pups per male was recorded at birth. In the breeding cage with the MTCH2^{f/f} male, 71 litters were counted, 46% females and 54% males. Only in one of the 5 cages with the BKO males, 4 pups were found. The pups died on the same day they were born. (B) A sperm aliquot was placed on a pre-warmed 100 μm deep counting chamber slide (Hamilton Thorne Research, Danvers, Mass.) and analyzed using a computer-assisted semen analysis (CASA) (Leja, Nieuw-Vennet, Netherlands). Parameters assessed were those recommended by the manufacturer for mouse sperm, and they are: total motility, progressive motility, rapid motility, VAP, average path velocity (μm/sec); VSL, straight line velocity (μm/sec); VCL, continuous line velocity (μm/sec); ALH, amplitude of lateral head displacement (μm); BCF, beat cross frequency (Hz); STR, straightness (ratio of VSL/VAP); LIN, linearity (ratio of VSL/VCL). Among the 50% motile spermatozoa in the wild-type, 25% of them are hyperactive. BKO sperm, on the other hand, have severe deficit in movement.

FIGS. 18A-I illustrate that BKO mice have severe testis degeneration. A-C: MTCH2f/f, D-I: BKO. A, B, D-G: ×20, C, H and I: ×40. HE stain. (A-C) MTCH2f/f: Normal testicular parenchyma. Many seminiferous tubules contain relatively mature stages of spermatozoa—seen as thin, oval nuclei close to the lumen (some are circled). Note the thickness of the layer composed of spermatogenic cells (double-headed arrows), irrespective of the specific maturation stage. Leydig cells located between the ST (asterisk). (D-I) BKO: Degenerative changes in seminiferous tubules. Relatively mature stages, with elongate dark nuclei are rare (circled in D). Multifocally, the thickness of the spermatogenic cells lining the ST is markedly reduced (arrows). Macrophages are present in the lumen (arrowheads) and there are scattered necrotic/apoptotic cells (curved arrows in G and H). The number of Leydig cells (asterisks) is normal.

FIGS. 19A-H illustrate that the number of mature spermatozoa is dramatically reduced in BKO males. A, B, E and F: MTCH2^{f/f} C, D, G and H: BKO. A, C, E and G: ×10. B, D, F and H: ×40. (A+B): MTCH2^{f/f}: Normal histologic features of the head of the epididymis. The tubules are lined by tall columnar cells. In the lumen there is a low number of mature spermatozoa (circled). (C+D): BKO: Most tubules are empty. A few contain rare spermatozoa (arrowheads) and cellular debris (arrows). MTCH2f/f: Normal histologic features of the tail of the epididymis. The tubules are lined by low columnar to flattened cuboidal cells. The lumen contains a large number of mature spermatozoa (circled) and rare presumptive macrophages (arrow). (G+H): BKO. The tubules are expanded by abundant eosinophilic fibrillar and granular debris, many presumptive macrophages (arrows) and a very low number of nuclei of mature spermatozoa (arrowheads).

FIG. 20 illustrates that LKO mitochondrial respiration rate is accelerated using complex II substrate. Mouse liver mitochondria (0.5 mg) were incubated in Clarke-type electrode chamber containing lml experimental buffer. To induce “state 3” respiration succinate+rotenone (10 mM and 20 μM, respectively) and 200 μM ADP were added to the chamber. To induce “state 4” respiration, 1 μM oligomycin was added following ADP withdrawal. Data are presented as mean±SEM of three independent experiments. *, P<0.05 versus WT control.

FIGS. 21A-B illustrate that liver knock-out (LKO) hepatocytes show an elevated rate of β-oxidation. (A) Schematic representation of the β-oxidation assay; FA oxidation was measured by incubation of primary hepatocytes isolated from LKO and WT mice with [9,10-³H]Myristate or [9,10-³H]Palmitate by measuring the amount of ³H₂O production. (B) LKO primary hepatocytes had higher beta oxidation rate both on [9,10-³H]Myristate and [9,10-³H]Palmitate FAs. Data are presented as mean±SEM of three independent experiments. *, P<0.05 versus WT control.

FIG. 22 illustrates that MTCH2 deletion causes an elevation of ATP content in mouse liver homogenates. Data are presented as mean±SD of four independent experiments. *, P<0.05 versus control.

FIGS. 23A-C illustrate that LKO mice are less sensitive to HFD feeding. (A) Average weight gain percentage of LKO mice and their WT littermates on ND (10% fat) and HFD (45% fat) for 23 weeks. Data are the means±S.E.M. (B) Individual mouse body weight gain 15 weeks following ND or HFD feeding. Data are the means±S.E.M, *P<0.05, by student t-test, indicating a significant difference between WT mice fed on ND versus HFD. (C) Serum TG levels of overnight fasted WT and LKO mice before and 14 weeks following HFD. Data are the means±S.E.M, *P<0.05, **P>0.005 by student t-test, indicating a significant difference between WT and LKO mice.

FIG. 24 illustrates shotgun lipidomic analysis of lipids from lipid extracts of mouse liver samples. Lipid extracts of hepatic samples from LKO (white bars) and WT mice (black bars) were prepared by using a modified Bligh and Dyer procedure. The molecular species of each lipid class in the lipid extracts were identified and quantified with an accuracy of 5-10% using an MDMS-SL technology as previously described. The total content of each lipid class is the summary of these quantified results.

FIGS. 25A-G illustrate that MTCH2 knockout results in an isolated growth defect without perturbing mESC differentiation and pluripotency. (A) MTCH2^{-/-} mESC fail to proliferate properly in vitro. 10⁵ cells from MTCH2^{F/F} and MTCH2^{-/-} clones were seeded on gelatin in 2i/LIF and were counted every 2 days. Results are presented as means±s.d. (n=3), *P<0.05. (B) MTCH2^{-/-} mESC fail to proliferate properly in vivo. Generating teratomas were done by injecting immune-compromised mice with MTCH2^{F/F} and MTCH2^{-/-} mESC clones (each clone into a separate leg of the same mouse). For both MTCH2^{F/F} and MTCH2^{-/-} clones, 5×10⁶ cells were injected side by side subcutaneously (S.C.) and were monitored for teratoma generation. The tumors were removed, weighed and photographed. Results are presented as means±s.d. (n=2), *P<0.05. (C)MTCH2^{-/-} mESC differentiate properly contributing to all 3 germ layers in vivo. After removal, the tumors were fixed and stained with H&E for histology analysis to detect cells representing all 3 germ layers. (D)MTCH2^{-/-} mESC secrete higher levels of lactate. Lactate secretion to the medium was measured using the NOVA Biomedical biotech analyzer. Results are presented as means ±s.d. (n=3), **P<0.005. (E) Schematics of labeled U-¹³C-glutamine metabolism by the TCA cycle using GC-MS. Abbreviations: Ac-CoA, acetyl-CoA; OAA, oxaloacetate; Gln, glutamine; Glu, glutamate; αKG, α-ketoglutarate; Succ-CoA, succinyl-CoA; Fum, fumarate; Mal, malate; PDH, pyruvate dehydrogenase; ACL, ATP-citrate lyase; IDH, isocitrate dehydrogenase; αKGDH, α-ketoglutarate dehydrogenase. (F) MTCH2^{-/-} mESC generate higher lactate levels (M+3) arising from uniformly labeled ¹³C-glucose. GC/MS analysis of lactate in cells cultured with L[U-¹³C]glucose for 6 hours is shown. Results are presented as means±s.d. (n=3), *P<0.05. (G) MTCH2^{-/-} mESC generate higher intermediary labeled metabolites arising from U-¹³Cs glutamine. Mass Isotopomer quantification of TCA cycle labeled intermediates in cells cultured with uniformly labeled ¹³C-glutamine is shown. Results are presented as means±s.d. (n=3), *P<0.05, **P<0.005.

### DESCRIPTION OF SPECIFIC EMBODIMENTS OF THE INVENTION

The present invention, in some embodiments thereof, relates to the treatment of diseases by regulating Mitochondrial carrier homologue 2 (MTCH2).

The principles and operation of the method of treating diseases according to the present invention may be better understood with reference to the Figures and accompanying descriptions. Mitochondrial carrier homologue 2 (MTCH2) is a 33 kDa protein belonging to the mitochondrial carrier (MC) protein family. It is an outer mitochondrial membrane protein with a critical function in the mitochondrial outer membrane permeabilization process by facilitating the recruitment of tBID to mitochondria.

Using haematopoietic-specific MTCH2 knockouts, the present inventors have surprisingly found that MTCH2 affects both stem cell mobilization and proliferation.

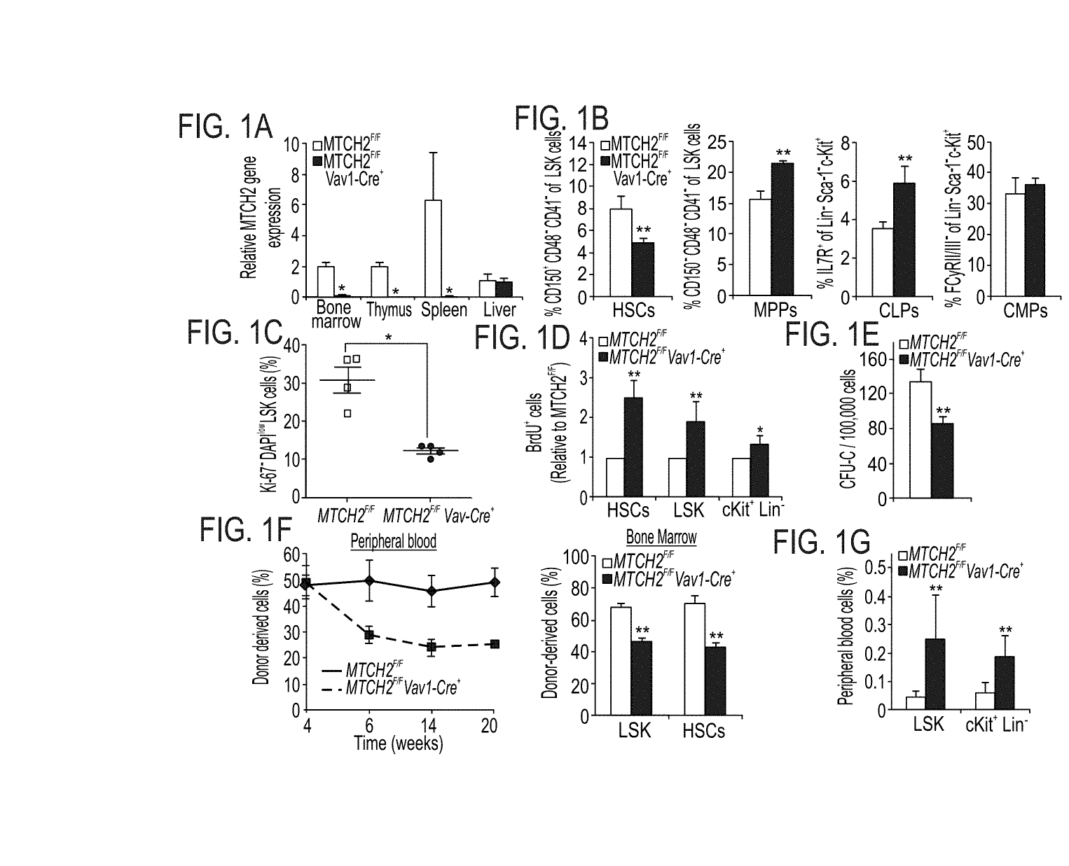

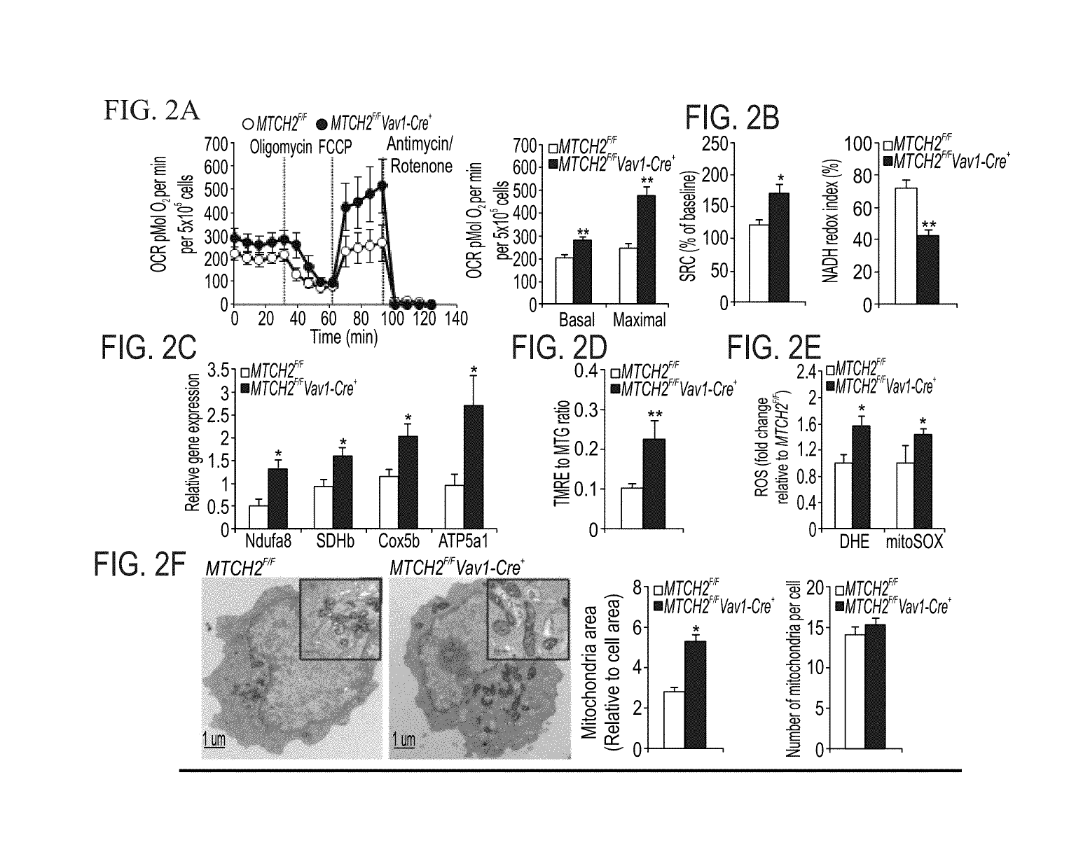

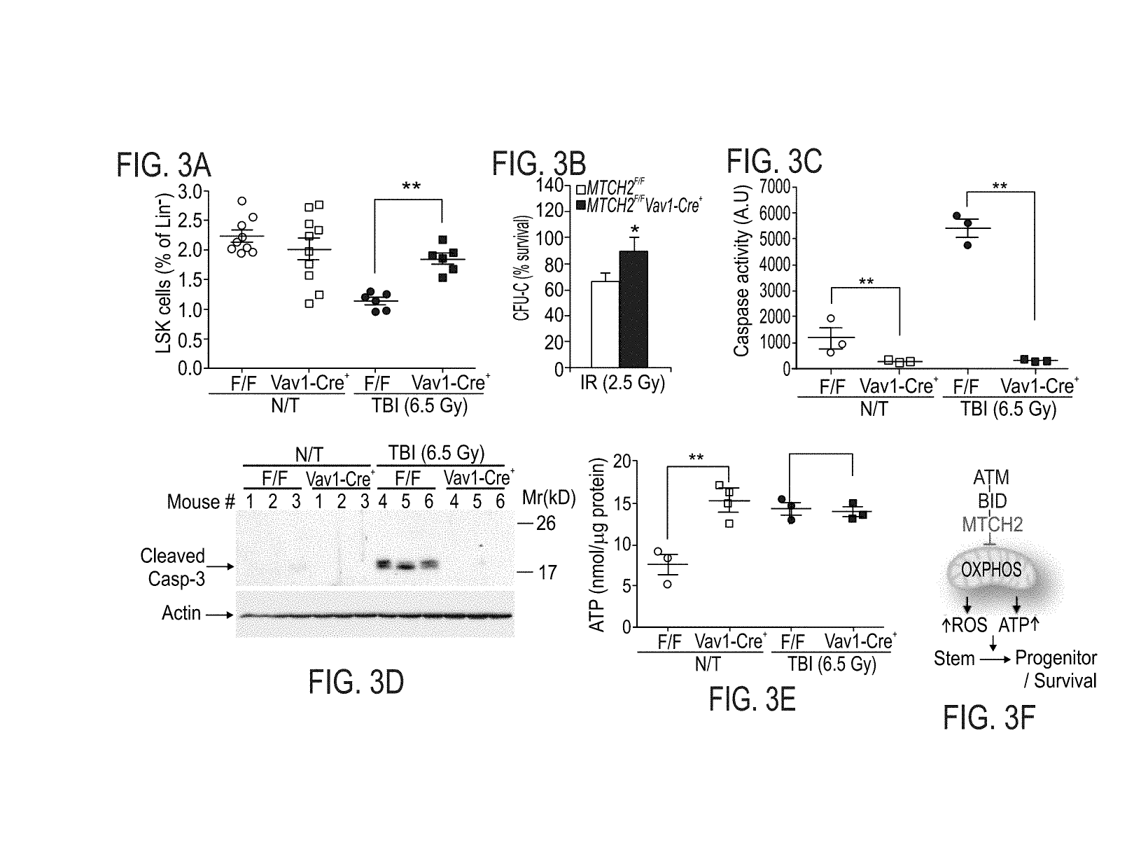

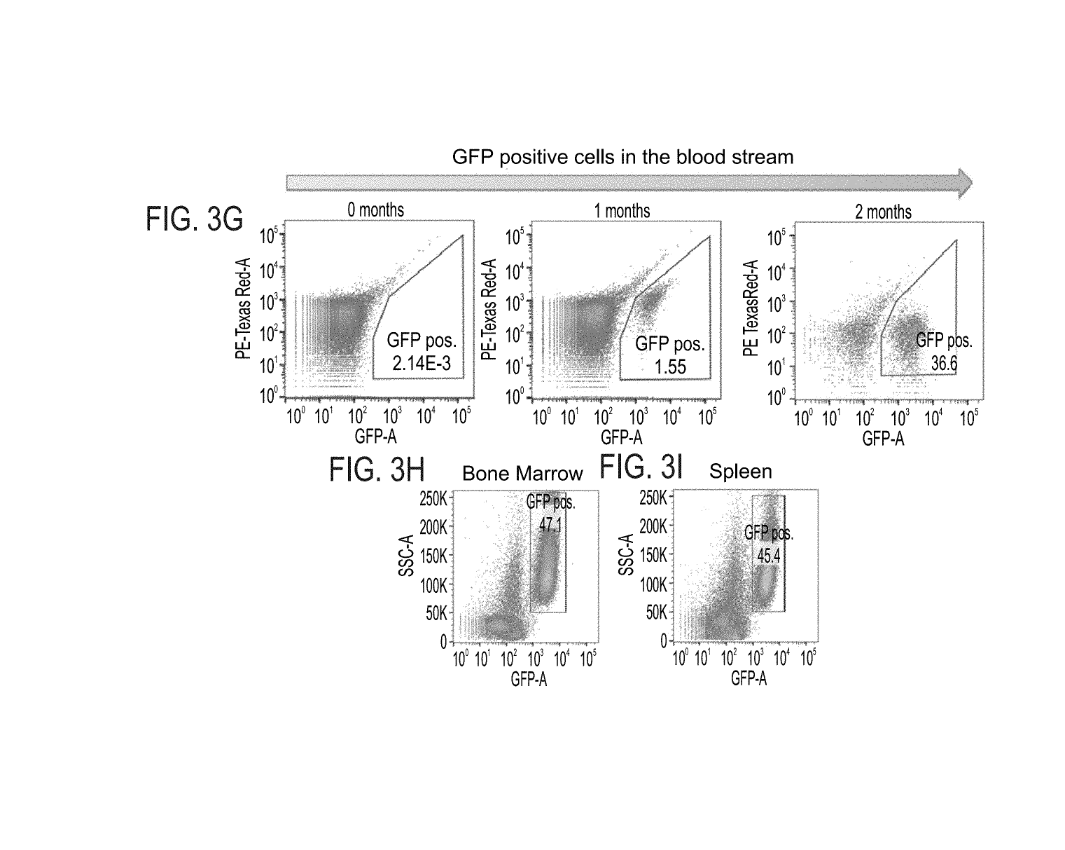

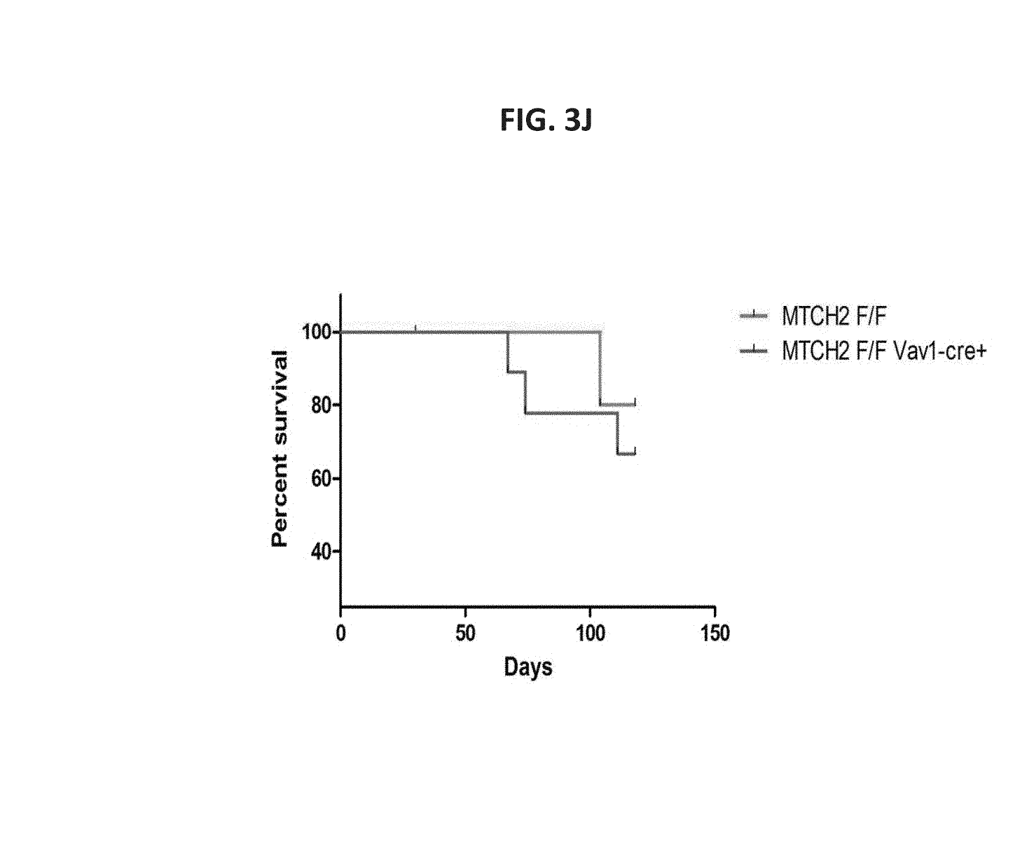

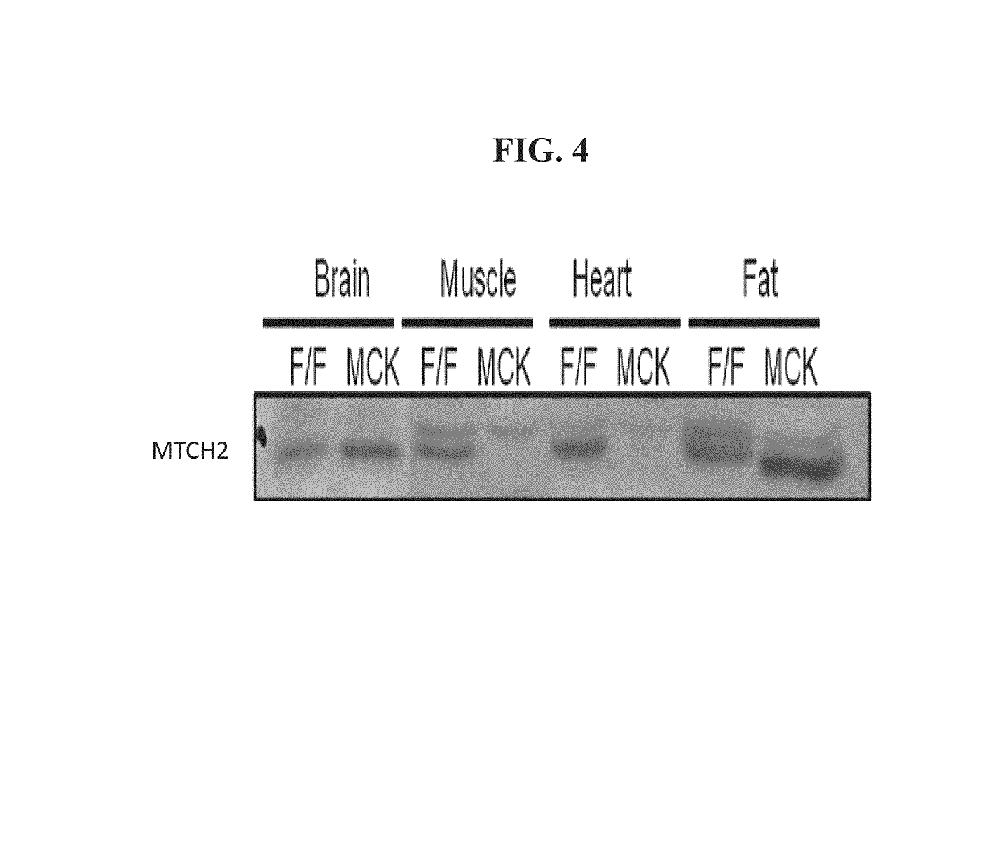

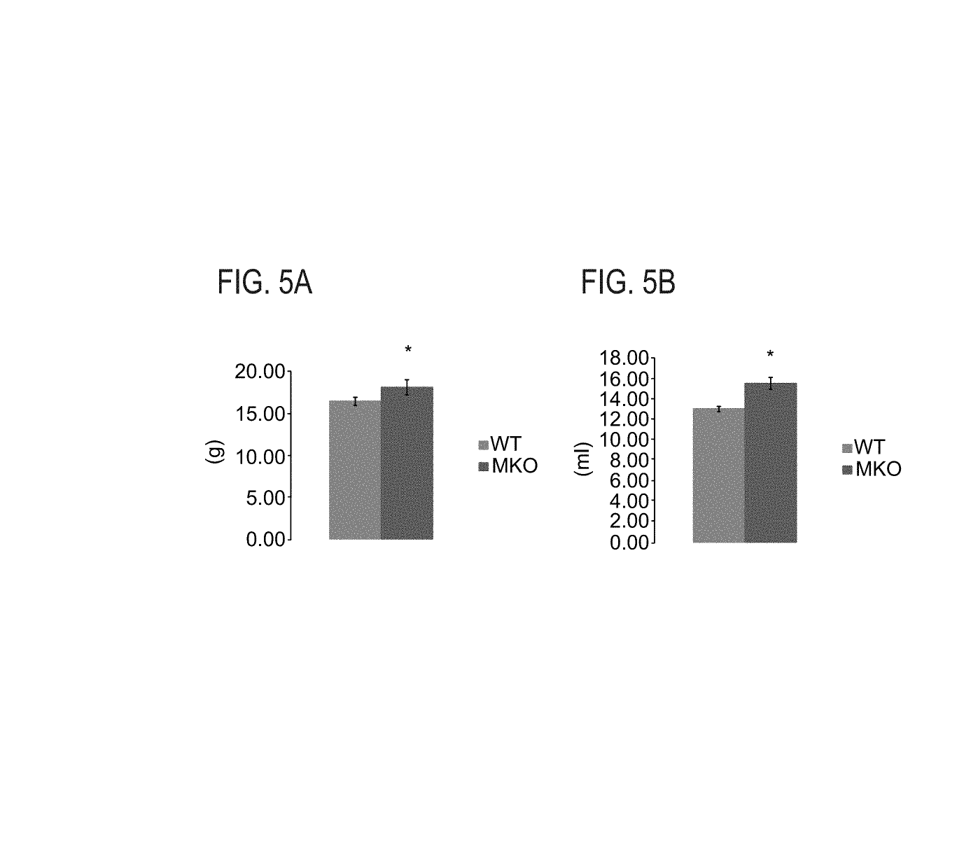

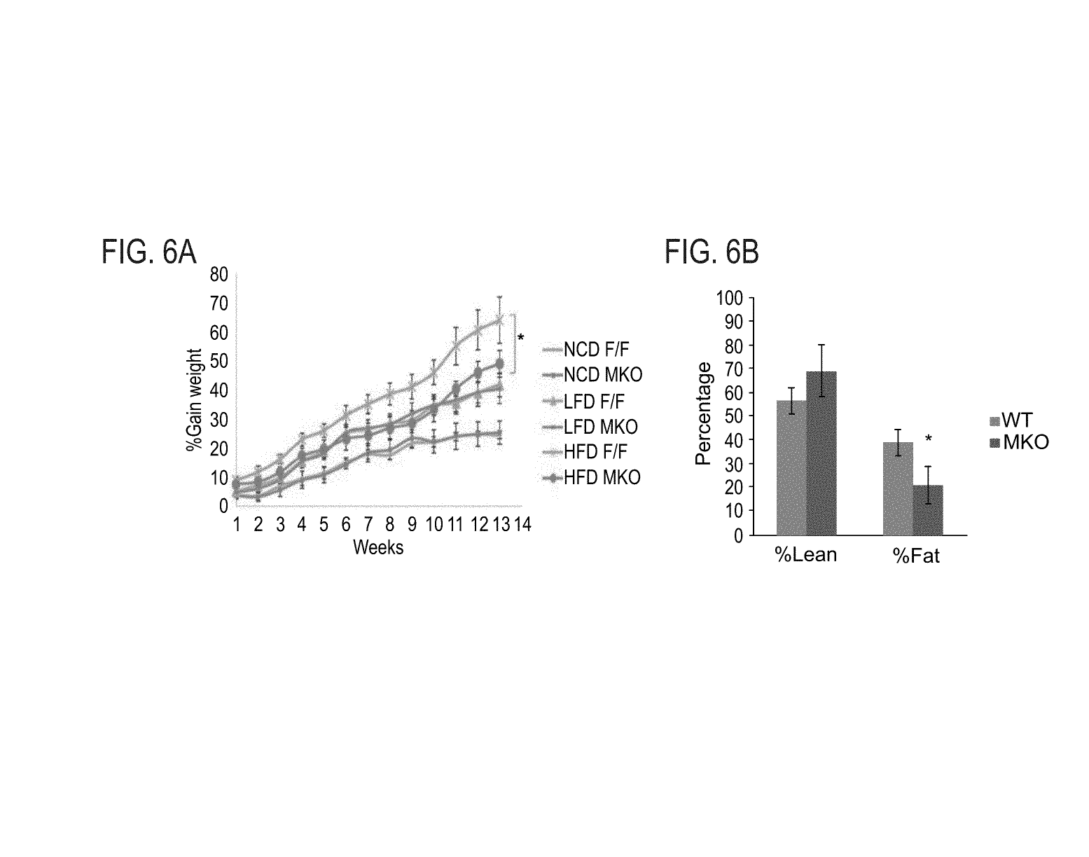

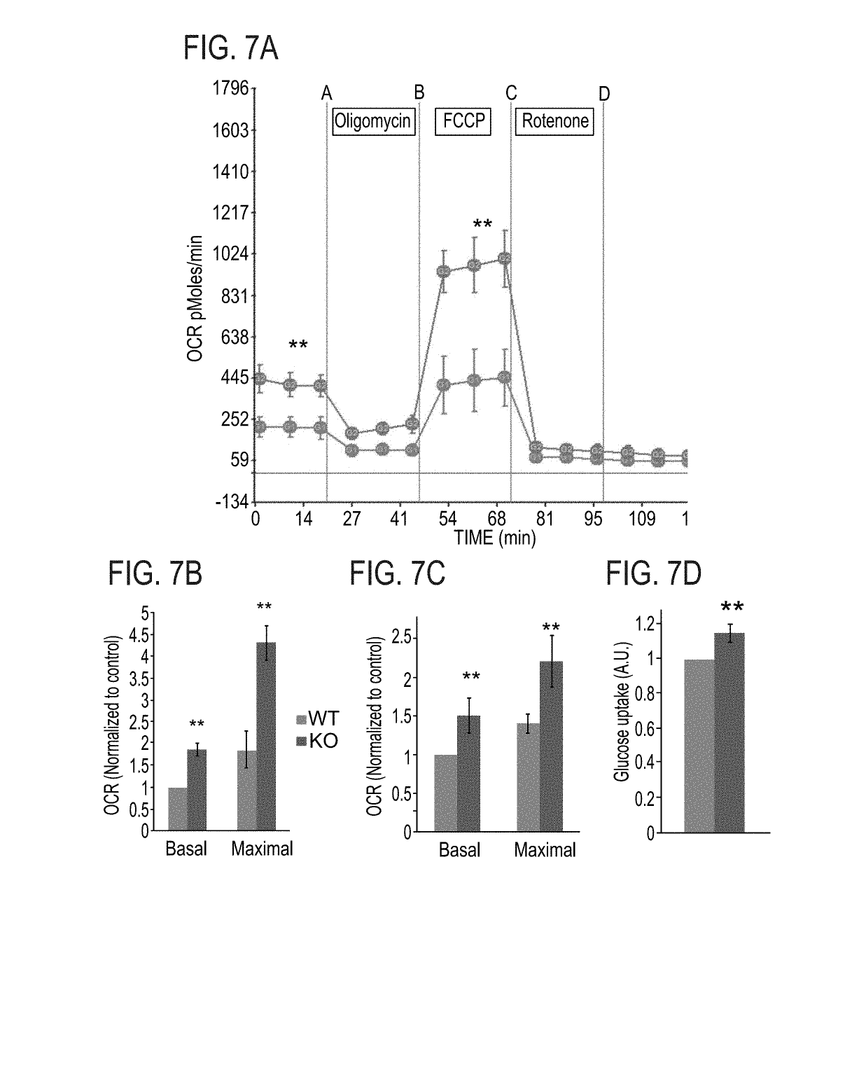

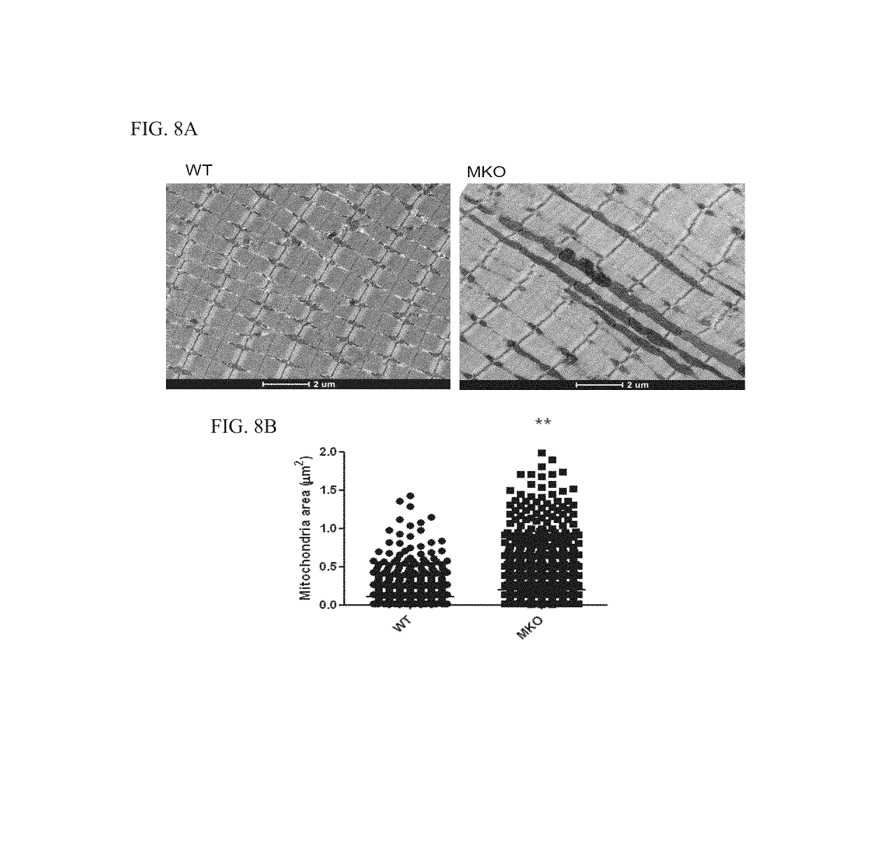

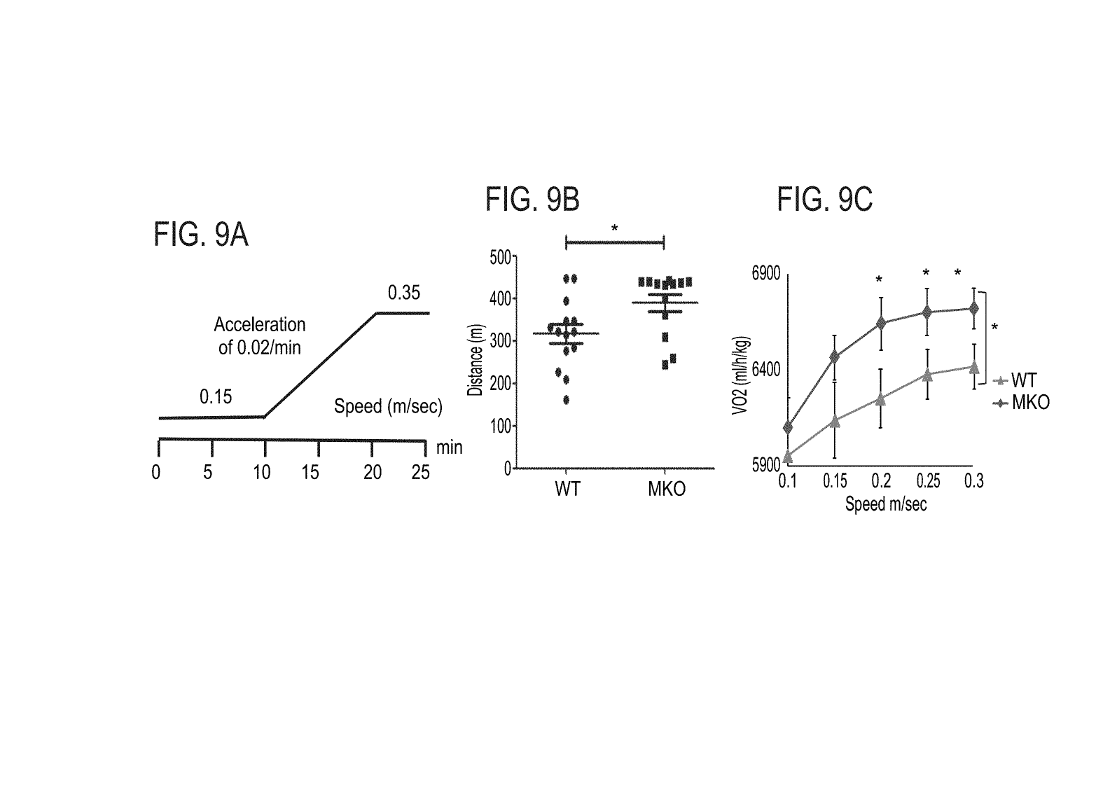

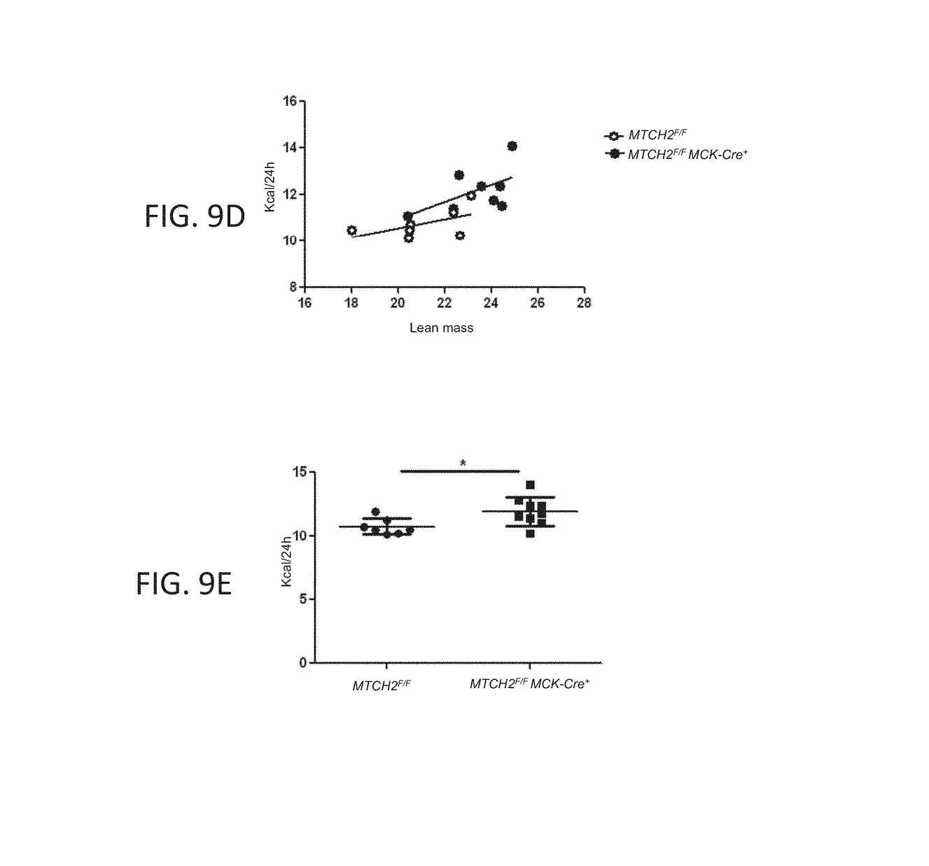

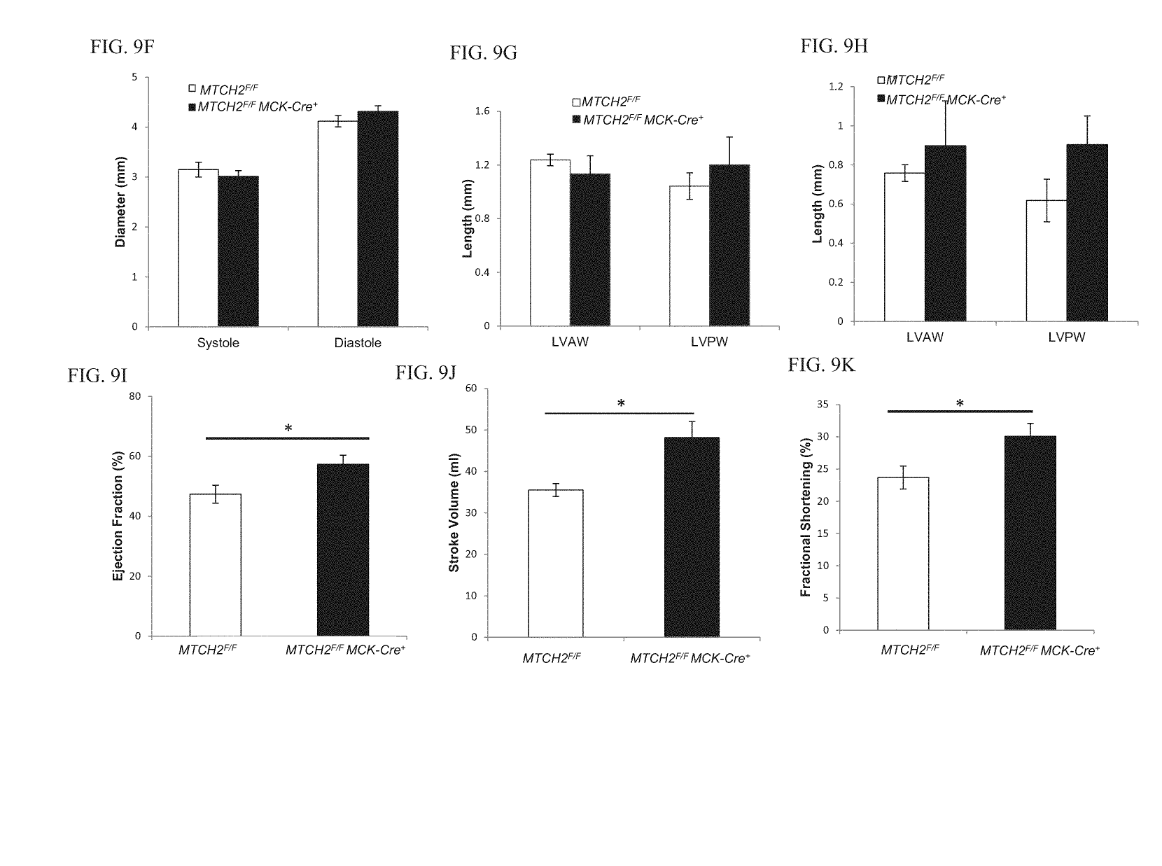

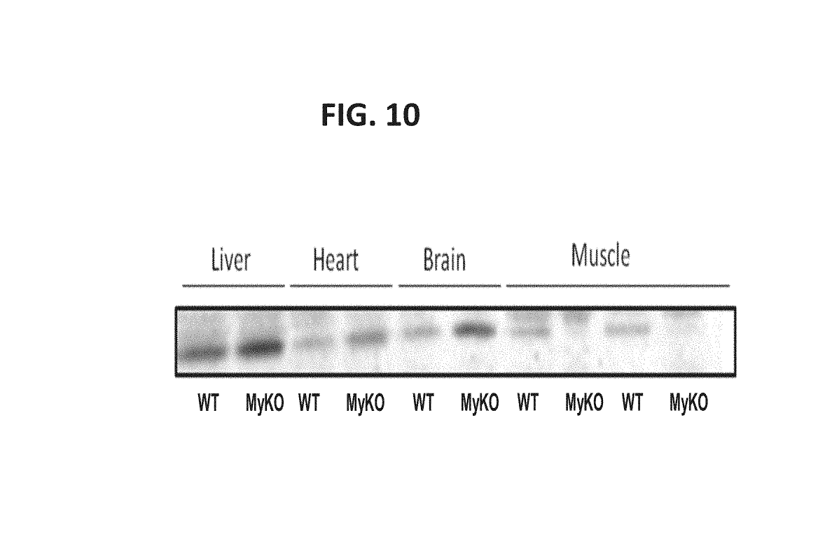

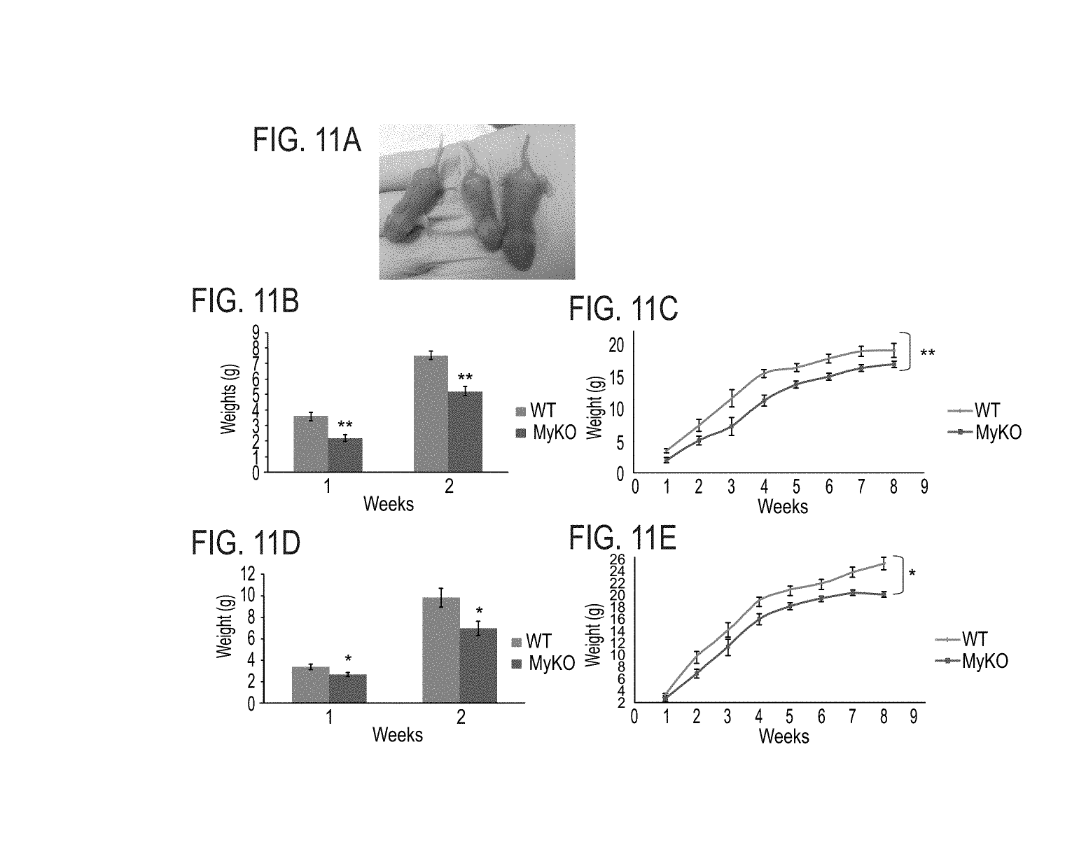

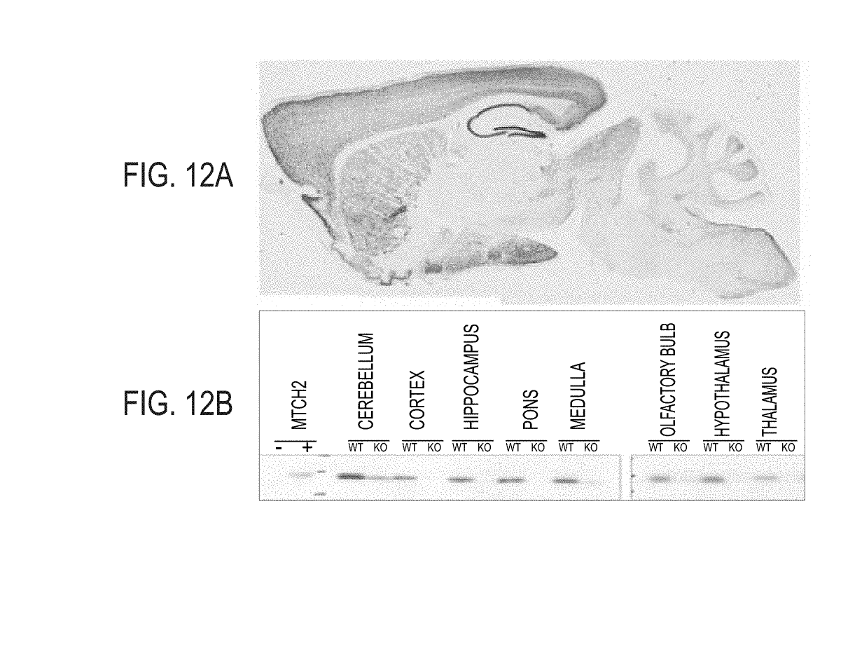

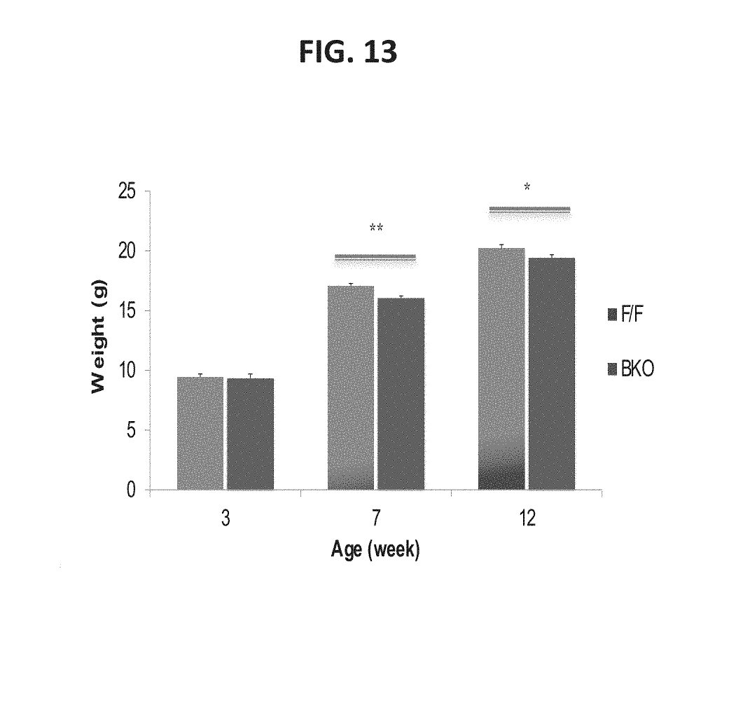

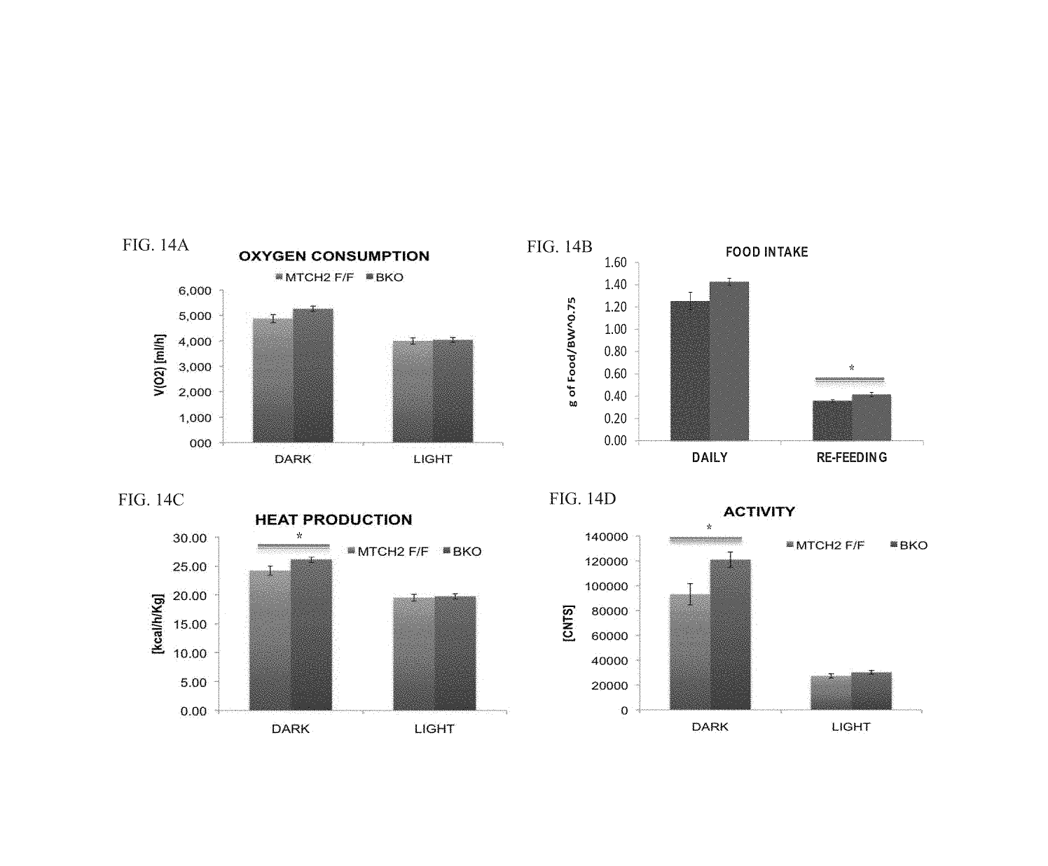

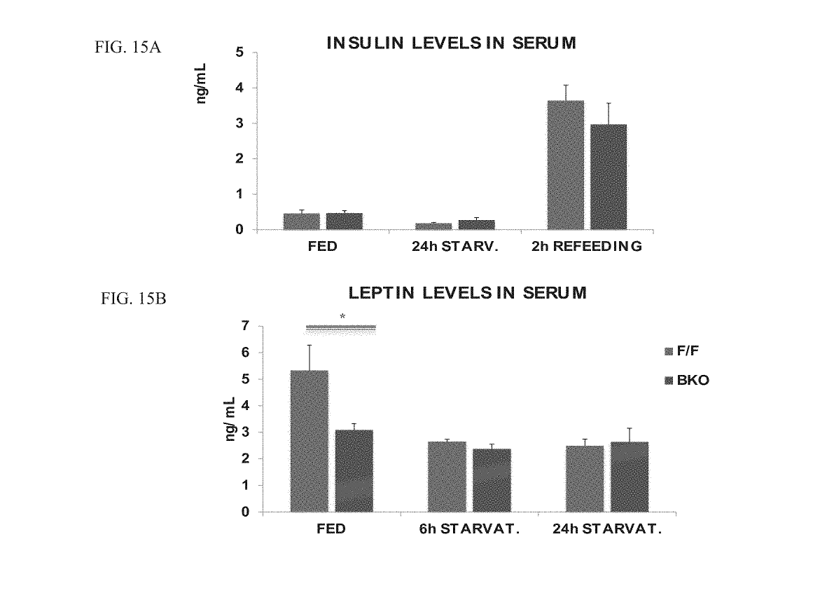

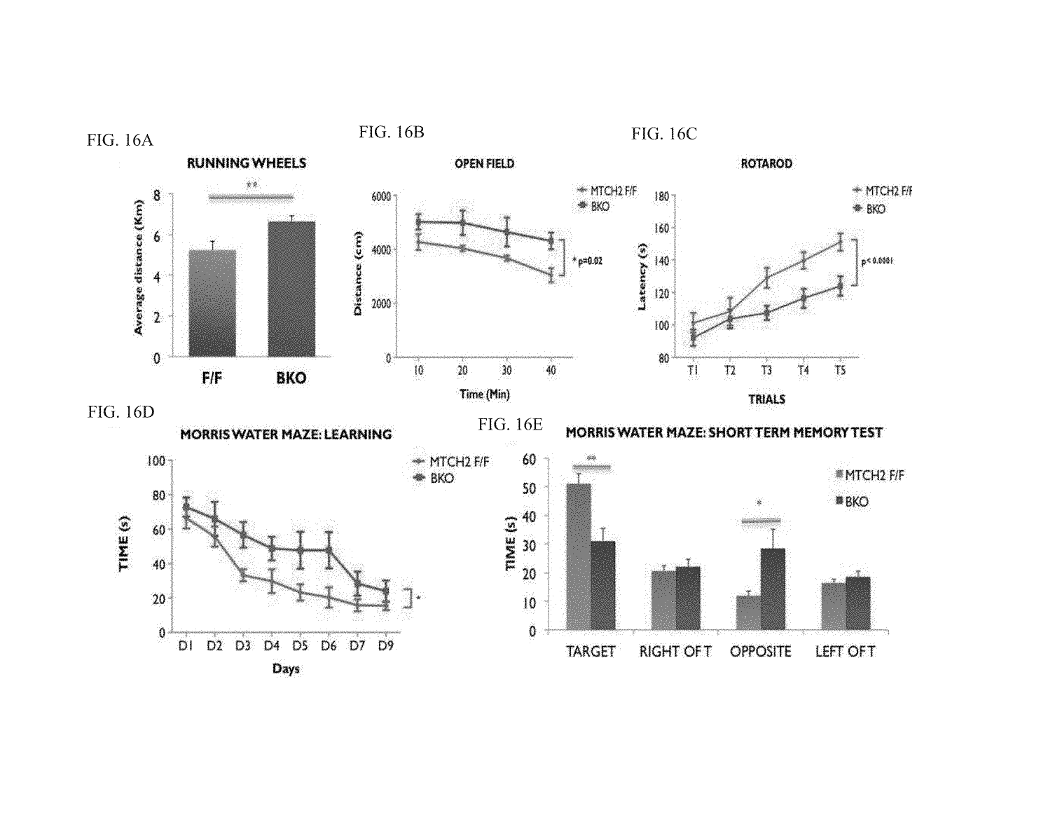

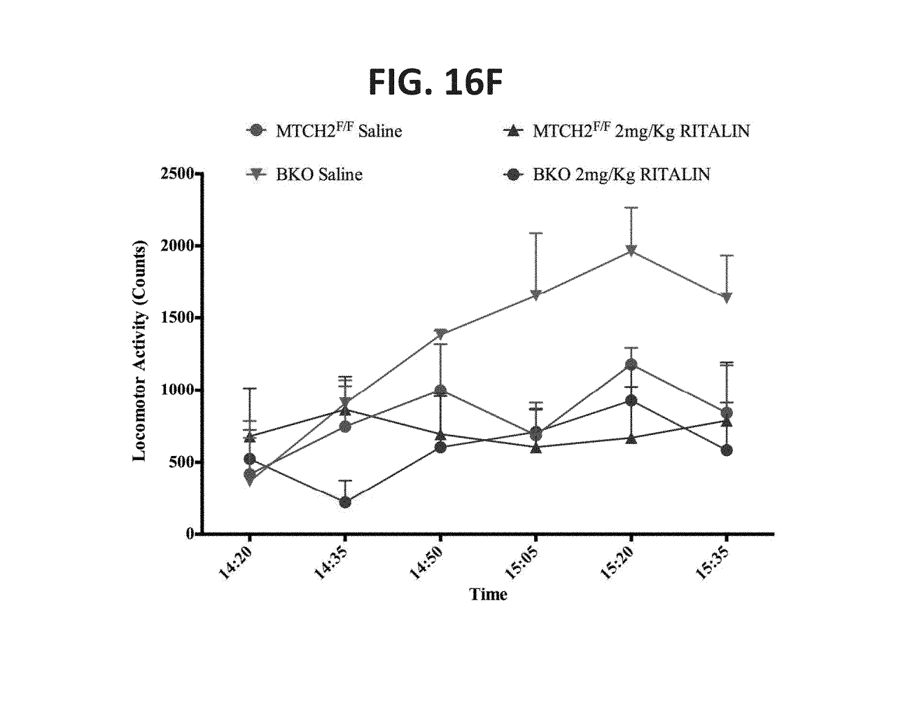

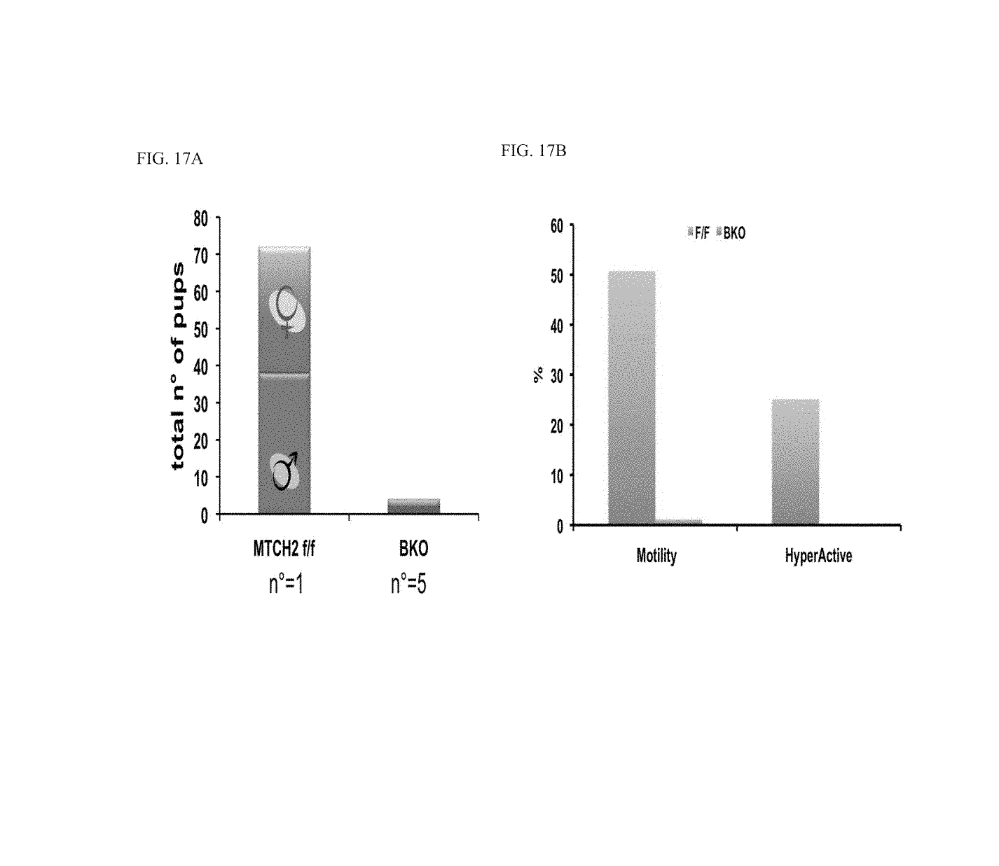

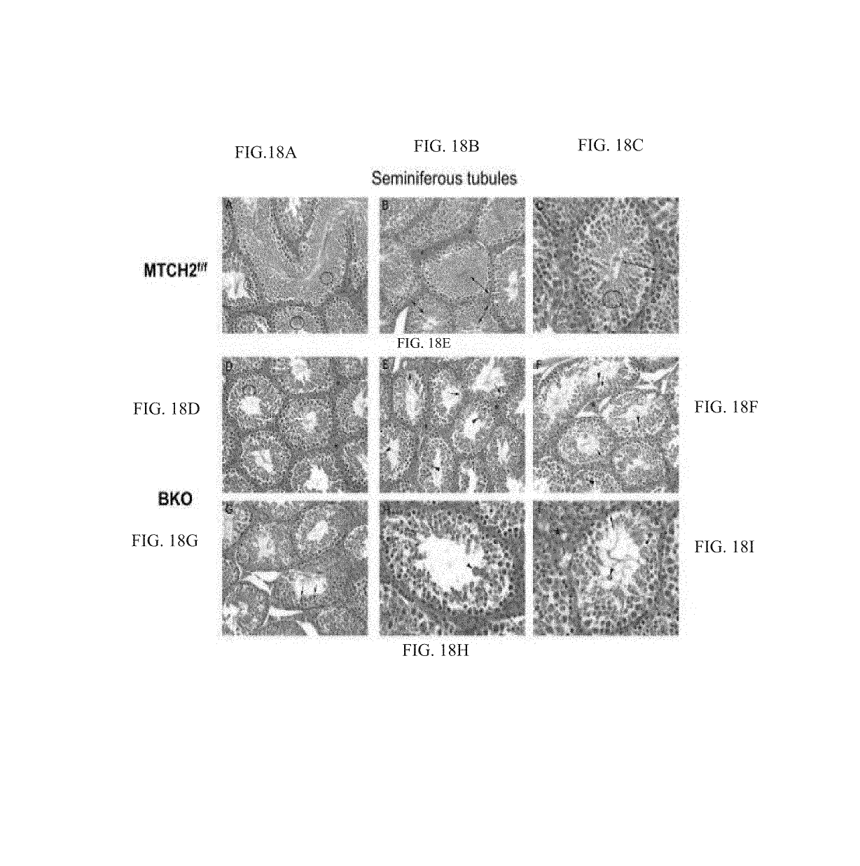

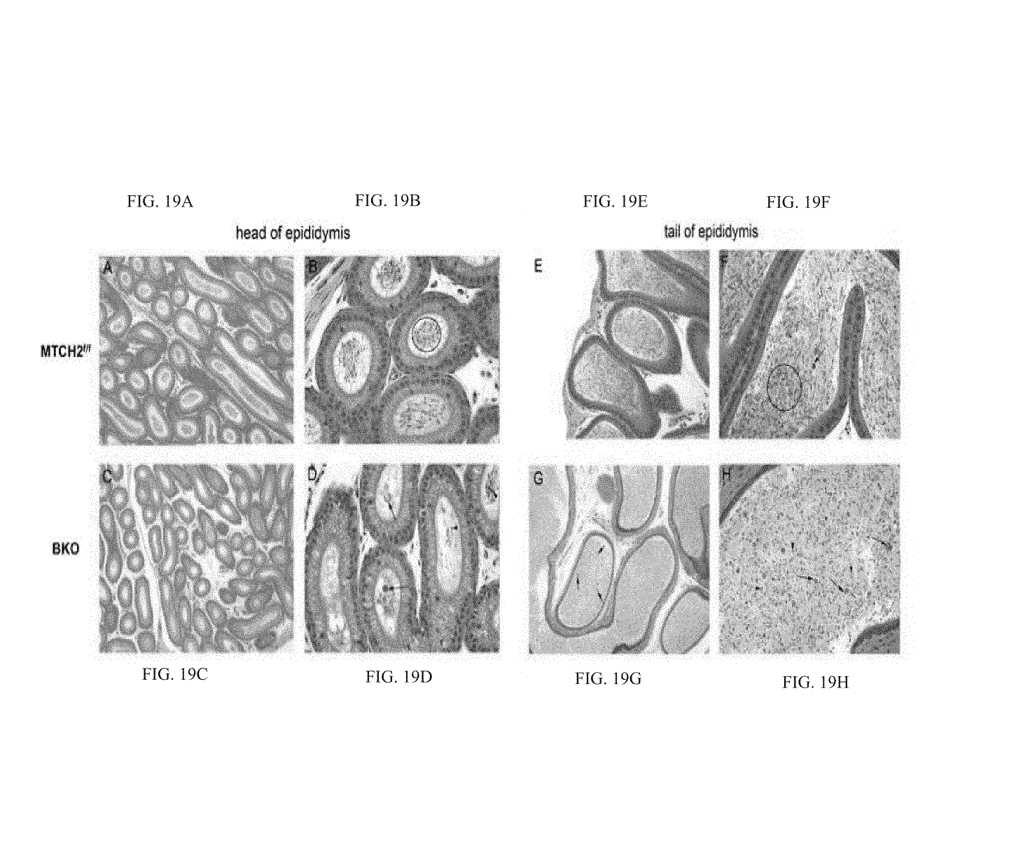

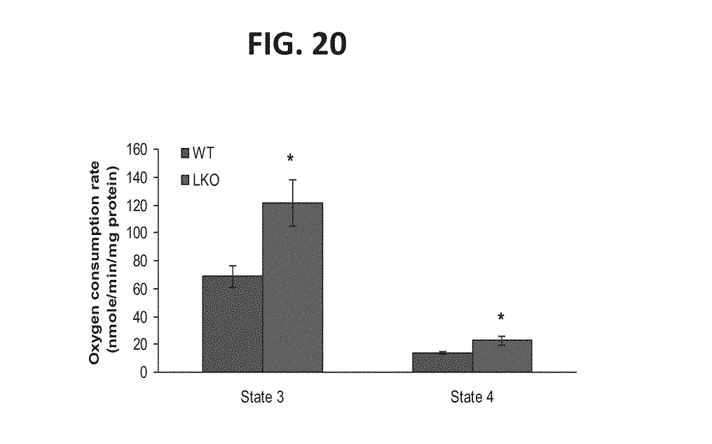

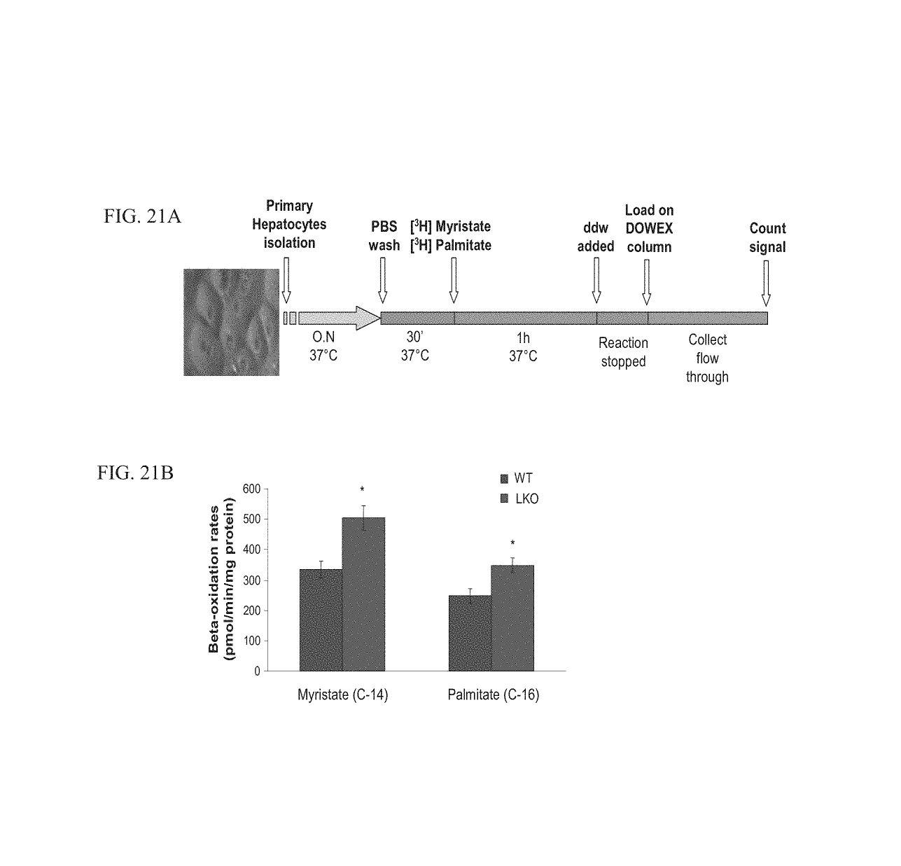

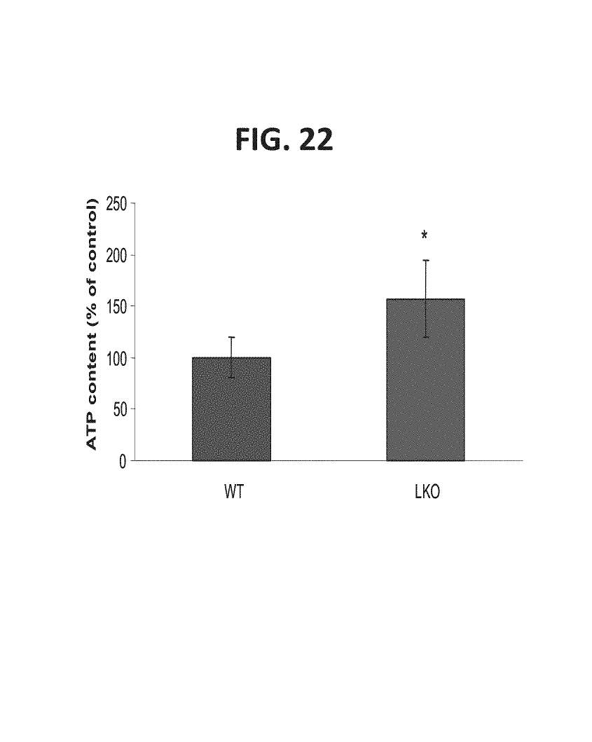

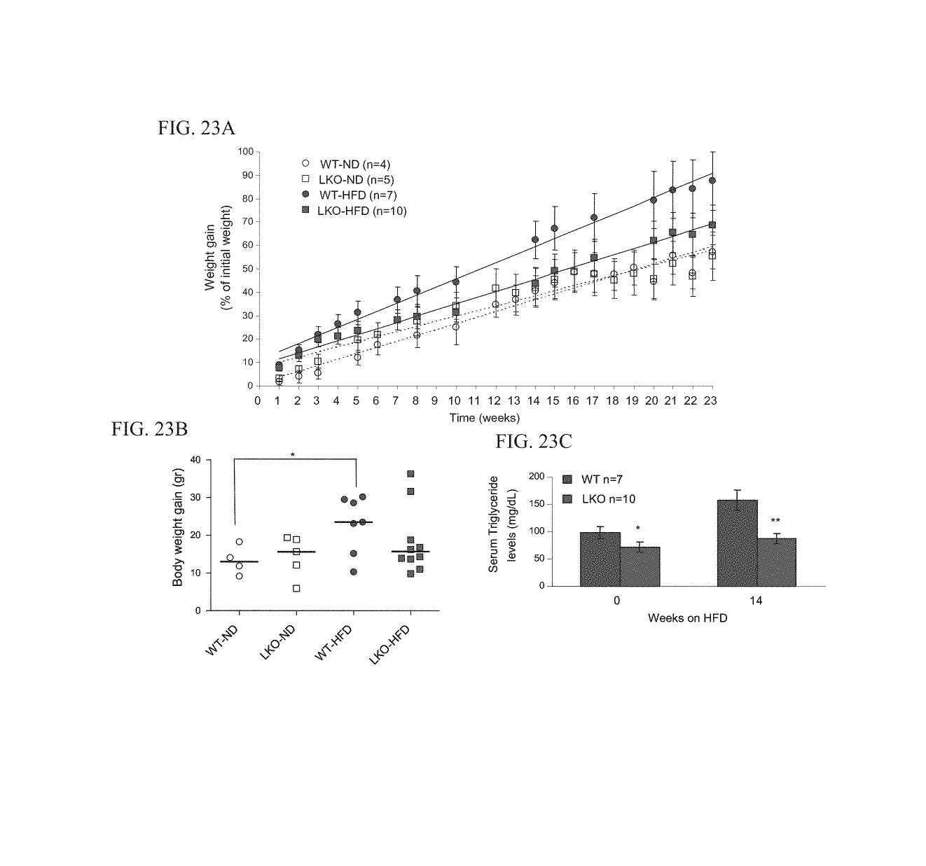

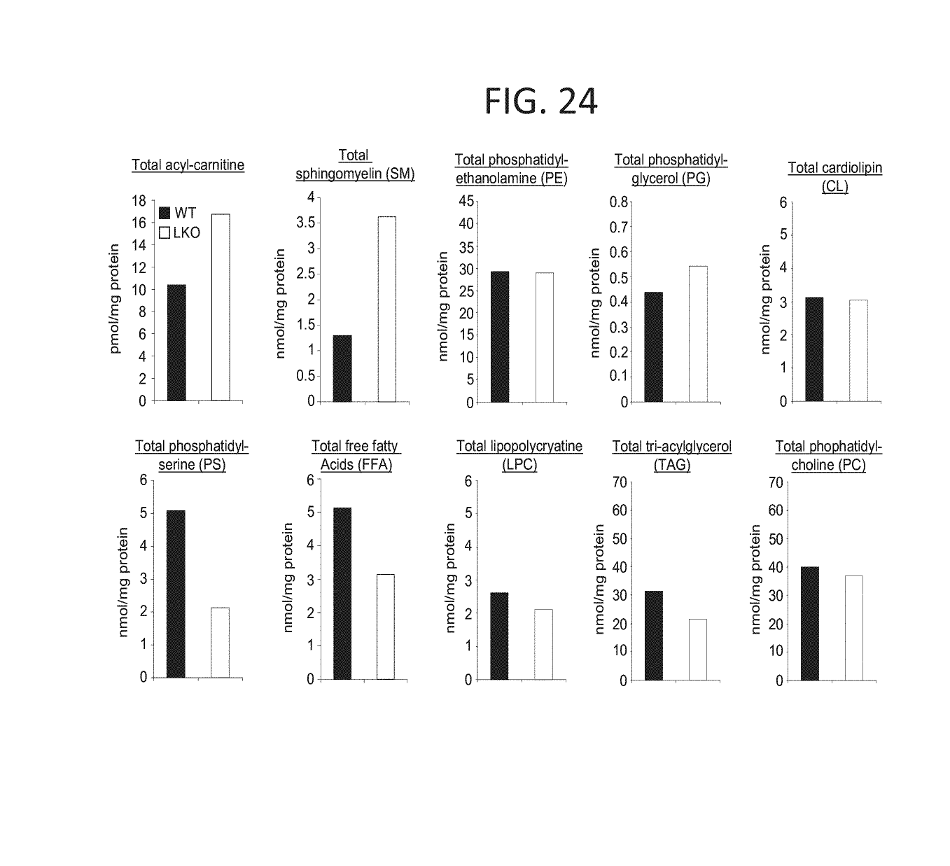

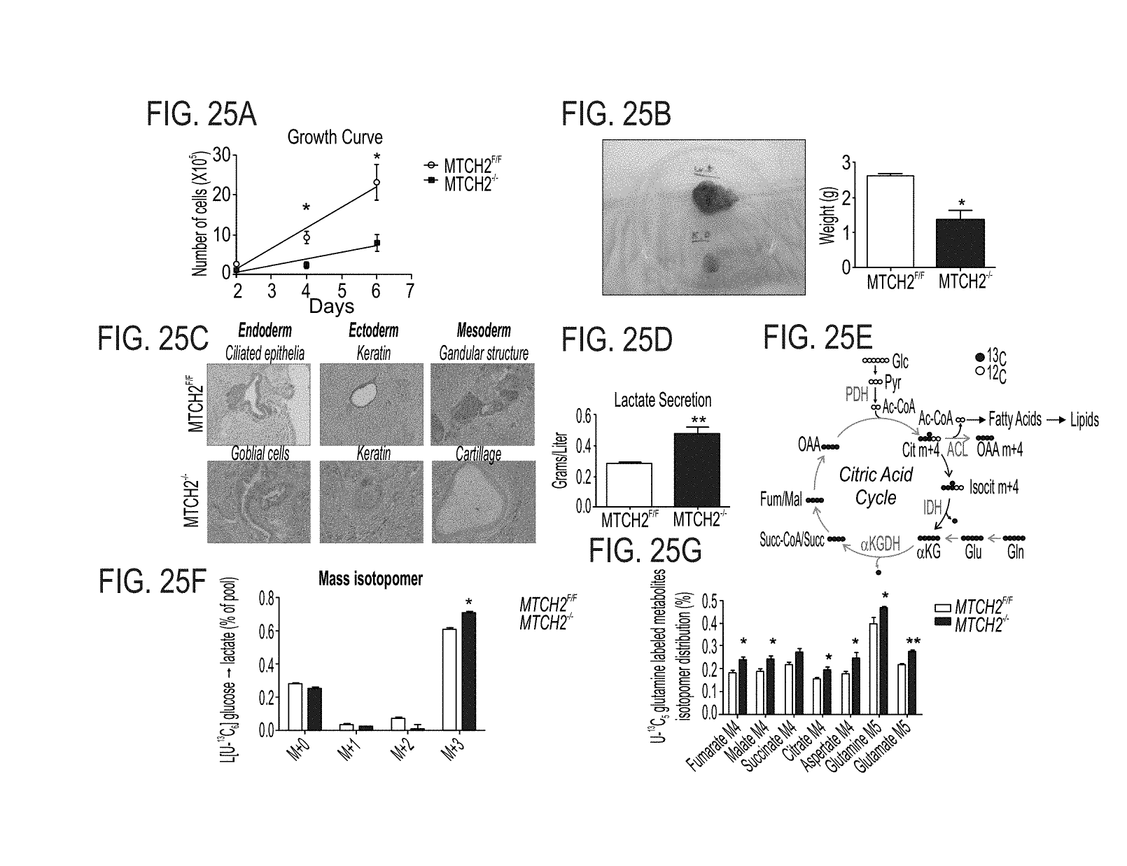

The present inventors showed that loss of mitochondrial carrier homolog 2 (MTCH2) primes mitochondrial oxidative phosphorylation which is sufficient to trigger hemapoietic stem cell (HSC) re-entry into cycle and sustain HSC viability in vivo (FIGS. 1A-G). Thus, the present inventors propose down-regulation of MTCH2 for the mobilization of hemapoietic stem cells.

In addition, MTCH2 knockout in mouse embryonic stem cells (mESC) results in impairment of proliferation and glucose/glutamine metabolism both in vitro and in vivo (FIG. 25A). No effect on the pluripotency of these cells was noted (FIG. 25B). Thus, the present inventors propose that in order to enhance proliferation of stem cells, without altering the differentiation potential, MTCH2 should be upregulated.

Whilst further reducing the present invention to practice, the present inventors analyzed the loss of MTCH2 in additional conditional knock-out mice.

When MTCH2 was knocked out selectively in the forebrain, the mice were shown to be hyperactive, have impaired motor planning and coordination as well as spatial memory disability. Thus, the present inventors propose that up-regulation of MTCH2 (and other agents which decrease mitochondrial metabolism such as metformin, nimodipine, memantine, oxytetracycline, amiodarone, sodium azide) may be an advantageous treatment for hyperactivity and other learning disabilities.

When MTCH2 was knocked out selectively in the liver, mice fed on a high fat diet were shown to gain less weight than the wild-type littermate mice on HFD, and their serum triglyceride levels were significantly lower than the levels in wild-type mice. Moreover, using shotgun lipidomics, it was found that MTCH2 deletion leads to a profound effect on the hepatic levels of fatty acids. Thus, the present inventors propose that down-regulation of MTCH2 may be an advantageous treatment for obesity and other fat-related disorders.

When MTCH2 was knocked out selectively in the skeletal muscle, mice were shown to gain less weight than their wild-type littermates and to show improved exercise endurance.

Furthermore, the mice were shown to have an increase in cardiac function.

Thus, the present inventors propose that down-regulation of MTCH2 may be an advantageous treatment for obesity, cardiac diseases and also to improve exercise endurance.

Thus, according to a first aspect of the present invention there is provided a method of increasing mobilization of hematopoietic precursors from the bone marrow to the peripheral blood in a subject in need thereof, the method comprising:
(a) administering to the subject an agent which downregulates an activity or expression of a component participating in the mitochondrial apoptotic pathway; and
(b) harvesting the hematopoietic precursors from the peripheral blood.

As used herein the term “mobilization” refers to the release of hematopoietic precursors (e.g., stem cells) from bone marrow into peripheral blood circulation.

As used herein “increasing mobilization” refers to inducing mobilization of peripheral blood precursor cells, to elevate circulating levels of peripheral blood precursor cells, or to enhance or facilitate hematopoietic reconstitution or engraftment, in a subject in need thereof.

As used herein “hematopoietic precursors” or “hematopoietic progenitor cells” refers to cells that, in response to certain stimuli, can form differentiated hematopoietic or myeloid cells. The presence of progenitor cells can be assessed by the ability of the cells in a sample to form colony-forming units of various types, including, for example, CFU-GM (colony-forming units, granulocyte-macrophage); CFU-GEMM (colony-forming units, multipotential); BFU-E (burst-forming units, erythroid); HPP-CFC (high proliferative potential colony-forming cells); or other types of differentiated colonies that can be obtained in culture using known protocols. As used herein, “stem” cells are less differentiated forms of progenitor or precursor cells. Typically, such cells are positive for CD34 in humans.

According to this aspect of the present invention, the term “subject” refers to a mammalian subject e.g., a human subject. The subject may be a healthy subject who serves as a donor for hematopoietic precursor transplantation. Alternatively, the subject may suffer from an immune deficiency and hence is in need of stem cell mobilization or organ e.g., stem cell transplantation (i.e., self-autologous; or from a donor i.e., non-autologous i.e., syngeneic, allogeneic or xenogeneic). In the latter case the subject is a recipient in need of an organ transplant.

Examples of immune deficiencies which can be treated according to the present teachings are provided below.

Typical conditions that can be ameliorated or otherwise benefited by cell mobilization or hematopoietic stem cell transplantation, include, but are not limited to, hematopoietic disorders, such as aplastic anemia, leukemias, drug-induced anemias, and hematopoietic deficits from chemotherapy or radiation therapy. The methods are also useful in enhancing the success of transplantation during and following immunosuppressive treatments as well as in effecting more efficient wound healing and treatment of bacterial inflammation. The methods are also useful for treating subjects who are immunocompromised or whose immune system is otherwise impaired. Typical conditions that are ameliorated or otherwise benefited by the method of the present invention, include those subjects who are infected with a retrovirus and more specifically who are infected with human immunodeficiency virus (HIV). The method thus targets a broad spectrum of conditions for which elevation of progenitor cells and/or stem cells in a subject would be beneficial or, where harvesting of progenitor cells and/or stem cell for subsequent stem cell transplantation would be beneficial. The compounds are also administered to regenerate myocardium by mobilizing bone marrow stem cells.

The methods described herein are also particularly suitable for those subjects in need of repeated or high doses of chemotherapy. For some cancer patients, hematopoietic toxicity frequently limits the opportunity for chemotherapy dose escalation or completion of prescribed chemotherapy. Repeated or high dose cycles of chemotherapy can be responsible for severe stem cell depletion leading to important long-term hematopoietic sequelae and marrow exhaustion. The methods of the present invention provide for improved mortality and blood cell count when used in conjunction with chemotherapy.

In other embodiments the hematological disorder is a hematologic malignancy/cancer such as leukemia and lymphoma. Other hematological cancers are further described herein below.

It will be appreciated that the mobilization of hemapoietic stem cells may result in elevation of the levels of leukocytes in peripheral blood of a subject. In one embodiment, the leukocytes are other than natural killer (NK) cells.

It will be appreciated that the mobilization of hemapoietic stem cells may result in elevation of the levels of neutrophils in peripheral blood of a subject.

It will be appreciated that the mobilization of hemapoietic stem cells may result in elevation of the levels of mononuclear cells in peripheral blood of a subject.

It will be appreciated that the mobilization of hemapoietic stem cells may result in elevation of the levels of monocytes in peripheral blood of a subject.

It will be appreciated that the mobilization of hemapoietic stem cells may result in elevation of the levels of mature or activated macrophages in peripheral blood of a subject.

It will be appreciated that the mobilization of hemapoietic stem cells may result in elevation of the levels of lymphocytes (e.g. B cells or T cells) in peripheral blood of a subject.

In one embodiment, the agent which down-regulates a component of the mitochondrial apoptotic pathway is used for promoting the recovery of the bone marrow depleted e.g. by irradiation or chemotherapy, for example in cancer patients undergoing cancer associated chemotherapy or bone marrow transplantation and patients with irradiation injuries. Thus in another embodiment, there is provided a method for elevating the levels of hematopoietic precursor cells in the bone marrow of a subject suffering from or at risk of bone marrow suppression associated with exposure to radiation or chemotherapy.

In another embodiment, the methods are useful for stimulating the proliferation of bone marrow hematopoietic precursors and mature cells.

In certain preferable embodiments, the agent which downregulates a component of the mitochondrial apoptotic pathway is administered to the subject in combination with one or more white blood cell mobilizing agents, e.g. progenitor and/or stem cell mobilizing agents, described in detail herein below. For example, the agents may be administered in concurrent or sequential combination with one or more other growth factors or cytokines that affect mobilization, for example colony stimulating factors (e.g. granulocyte-colony stimulating factor, G-CSF and granulocyte-macrophages colony stimulating factor, GM-CSF) and stem cell factor, SCF). In other preferable embodiments, the agent is administered in combination with G-CSF.

In other embodiment, the compositions of the invention are particularly useful for the treatment of cytopenia, e.g. neutropenia.

The term “cytopenia” as used herein refers to a reduction of cellular elements in the circulating blood. Cytopenia may result from a variety of causes, and include both a general reduction of cell numbers in the blood as well as a specific reduction of a particular cell type, such as leukocyte reduction in leukopenia. Leukopenia is a reduction in the circulating WBC count to <4000/μL. It is usually characterized by a reduced number of circulating neutrophils, although a reduced number of lymphocytes, monocytes, eosinophils, or basophils may also contribute. Thus, immune function is generally greatly decreased. Neutropenia is a reduction in blood neutrophil count. Severe neutropenia is usually defined by an absolute neutrophil count <500/μL. It is more serious when accompanied by monocytopenia and lymphocytopenia. Lymphocytopenia, in which the total number of lymphocytes is <1000/μL in adults, is not always reflected in the total WBC count, because lymphocytes account for only 20 to 40% of the count.

In some embodiments, the subject is suffering from neutropenia associated with high dose chemotherapy, neutropenia associated with conventional oncology therapy, drug-induced neutropenia, toxin-induced neutropenia, and radiation-induced neutropenia. In certain other embodiments, the duration of neutropenia is reduced below 12 days, preferably below 10 days, more preferably below 8 days and most preferably below 7 days from the onset of treatment. In other embodiments, the duration of neutropenia is reduced below 5 days, preferably below 4 days, more preferably below 3 days, more preferably below 2 days and most preferably below one day from the onset of treatment. In another embodiment, the neutropenia is febrile neutropenia.

In another embodiment, the methods are useful for reducing the incidence of infection and for increasing survival following chemotherapy or radiation therapy in cancer patients.

As mentioned, the method according to this aspect of the present invention is performed by administering to the subject an amount of an agent which downregulates an activity or expression of a component participating in the mitochondrial apoptotic pathway.

Mitochondrial Apoptotic Pathway

As used herein, the phrase “a component of the mitochondrial apoptotic pathway” refers to a polypeptide or polynucleotide involved in the mitochondrial apoptotic pathway. According to one embodiment, the component is MTCH2 or a component which is upstream or downstream to MTCH2. Exemplary components are described herein below.

The BH3-only BID protein is a critical activator of the mitochondrial apoptotic pathway. Activation of the tumor necrosis factor (TNF)/Fas death receptors leads to caspase-8-mediated cleavage of BID to the truncated form, tBID, that translocates to the mitochondria to activate Bax and Bak, resulting in mitochondrial outer membrane permeabilization (MOMP).

The mitochondrial carrier homolog 2 (MTCH2/MIMP) [also called met-induced mitochondrial protein (MIMP)] is an evolutionary conserved protein, which carries six α-helixes that cross the outer mitochondrial membrane and interacts with the activated form of the BH3-only protein BID (tBID) in cells signaled to die by tumor necrosis factor-alpha (TNFα) or FAS.

According to a particular embodiment, the component which is regulated is MTCH-2 (human MTCH2 (GenBank Accession No. NP_055157; SEQ ID NO: 11) or an agonist thereof (e.g. tBID).

The term “tBID” refers to the C-terminal truncated fragment of the BH3 interacting death agonist (BID) protein which results from the enzymatic cleavage of cytosolic BID (e.g., by active caspase). As is mentioned in the background section, at an early stage of apoptosis, tBID translocates to the mitochondria and mediates the release of Cyt c therefrom. Non-limiting examples of tBID proteins include the mouse tBID (amino acids 60-195 of SEQ ID NO: 12; GenBank Accession No. AAC71064) and human tBID (amino acids 61-195 of SEQ ID NO: 13; GenBank Accession No. CAG30275).

The present invention also contemplates kits for mobilization of hemapoietic stem cells which comprise the agent which downregulates an activity or expression of a component participating in the mitochondrial apoptotic pathway together with a mobilization factor.

According to a particular embodiment, the mobilization factor is a drug such as cyclophosphamide or 5-fluorouracil; and/or certain antibodies, such as anti-VLA4. Additional examples of mobilization factors which can be included in the kit include but are not limited to, Granulocyte-colony stimulating factor (G-CSF) or granulocyte-macrophage colony stimulating factor (GM-CSF) (sargramostim, Berlex, Richmond, Calif.), most frequently employed in the clinic, efficiently mobilizes HSPC after a few consecutive daily injections. Erythropoietin, now commonly used among cancer patients undergoing chemotherapy to maintain hemoglobin in the near normal range, also has some ability to mobilize CD34⁺ cells. Stem cell factor (SCF) has been shown to be an excellent mobilizing agent, particularly when used in combination with G-CSF. Other compounds, such as polysaccharides (e.g., zymosan), mobilize HSPC within 1 hour after a single injection(10). Mobilization could also be induced by chemokines (e.g., IL-8, Gro-β), growth factors (e.g., vascular endothelial growth factor), and CXCR4 antagonists. Longer lasting variants of G-CSF (pegfilgrastim, Amgen) and erythropoietin (darbopoietin, Amgen) are now available and are in clinical trials as mobilizing agents. They have the benefit of very long half-lives and so add an important measure of patient convenience and the probability that timing of collection may be more flexible without sacrificing optimal collections.

Examples of CXCR4 antagonists include Mozobil (plerixafor) (AnorMED Inc.), AMD-070 (AnorMED Inc.), BKT140 (Biokine Therapeutics Inc.), CXCR4 monoclonal antibody (Northwest Biotherapeutics Inc.), KRH-2731/CS-3955 (Daiichi Sankyo Company), AVR 118 (reticulose) (Advanced Viral Research Corp.), CXCR4 antagonist (TaiGen Biotechnology), and CTCE-0214 (Chemokine Therapeutics Corp). A new factor (AMD3100, AnorMed, Vancouver, Canada), which is a reversible inhibitor of the binding of stromal derived factor (SDF-1a) to its cognate receptor CXCR4, is currently in clinical trials as a mobilizing agent. It is the first agent to be tried for mobilization based on a rational understanding of its mechanism of action relative to HPC-stromal cell interactions (see Section I). While it mobilizes CD34⁺ cells adequately on its own, it significantly improves the mobilization capacity of G-CSF when used in combination with G-CSF in mice. Clinical trials in humans with various diseases are in progress, including myeloma.

As mentioned herein above, the present inventors have uncovered that manipulation of MTCH2 effect the proliferation of stem cells in culture without affecting their differentiation potential.

Thus, according to another aspect of the present invention there is provided a method of propagating stem cells comprising culturing the stem cells in a culture medium comprising an agent that upregulates an activity and/or expression of a component participating in the mitochondrial apoptotic pathway under conditions that allow propagation of the stem cells, but do not allow differentiation of the stem cells, thereby propagating the stem cells.

As used herein, the phrase “stem cells” refers to cells which are capable of remaining in an undifferentiated state (e.g., pluripotent or multipotent stem cells) for extended periods of time in culture until induced to differentiate into other cell types having a particular, specialized function (e.g., fully differentiated cells). Preferably, the phrase “stem cells” encompasses embryonic stem cells (ESCs), induced pluripotent stem cells (iPS), adult stem cells and hematopoietic stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763) and embryonic germ (EG) cells which are obtained from the genital tissue of a fetus any time during gestation, preferably before 10 weeks of gestation.

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc in a somatic stem cell.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

Hematopoietic stem cells, which may also referred to as adult tissue stem cells, include stem cells obtained from blood or bone marrow tissue of an individual at any age or from cord blood of a newborn individual.

Preferred stem cells according to this aspect of some embodiments of the invention are embryonic stem cells, preferably of a human or primate (e.g., monkey) origin.

Placental and cord blood stem cells may also be referred to as “young stem cells”.

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used according to some embodiments of the invention. Human ES cells can be purchased from the NIH human embryonic stem cells registry [Hypertext Transfer Protocol://grants (dot) nih (dot) gov/stem_cells/registry/current (dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Eneavour-2, WIBR1, WIBR2, WIBR3, WIBR4, WIBR5, WIBR6, HUES 45, Shef 3, Shef 6, BJNhem19, BJNhem20, SA001, SA001.

In addition, ES cells can be obtained from other species as well, including mouse (Mills and Bradley, 2001), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 February 2008. This method comprises removing a single cell from an embryo during an in vitro fertilization process. The embryo is not destroyed in this process.

EG cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S., Cell Stem Cell. 2007, 1(1):39-49; Aoi T., et al., Generation of Pluripotent Stem Cells from Adult Mouse Liver and Stomach Cells. Science. 2008 Feb 14. (Epub ahead of print); I H Park, Zhao R., West J. A., et al. Reprogramming of human somatic cells to pluripotency with defined factors. Nature 2008;451:141-146; K. Takahashi, Tanabe K., Ohnuki M., et al. Induction of pluripotent stem cells from adult human fibroblasts by defined factors. Cell 2007;131:861-872]. Other embryonic-like stem cells can be generated by nuclear transfer to oocytes, fusion with embryonic stem cells or nuclear transfer into zygotes if the recipient cells are arrested in mitosis.

Adult tissue stem cells can be isolated using various methods known in the art such as those disclosed by Alison, M. R. [J Pathol. 2003 200(5): 547-50], Cal, J. et al., [Blood Cells Mol Dis. 2003 31(1): 18-27], Collins, A. T. et al., [J Cell Sci. 2001; 114(Pt 21): 3865-72], Potten, C. S. and Morris, R. J. [Epithelial stem cells in vivo. 1988. J. Cell Sci. Suppl. 10, 45-62], Dominici, M. et al., [J. Biol. Regul. Homeost. Agents. 2001, 15: 28-37], Caplan and Haynesworth [U.S. Pat. No. 5,486,359] Jones E. A. et al., [Arthritis Rheum. 2002, 46(12): 3349-60]. Fetal stem cells can be isolated using various methods known in the art such as those disclosed by Eventov-Friedman S, et al., PLoS Med. 2006, 3: e215; Eventov-Friedman S, et al., Proc Natl Acad Sci USA. 2005, 102: 2928-33; Dekel B, et al., 2003, Nat Med. 9: 53-60; and Dekel B, et al., 2002, J. Am. Soc. Nephrol. 13: 977-90. Hematopoietic stem cells can be isolated using various methods known in the arts such as those disclosed by “Handbook of Stem Cells” edit by Robert Lanze, Elsevier Academic Press, 2004, Chapter 54, pp 609-614, isolation and characterization of hematopoietic stem cells, by Gerald J Spangrude and William B Stayton.

Generally, isolation of adult tissue stem cells is based on the discrete location (or niche) of each cell type included in the adult tissue, i.e., the stem cells, the transit amplifying cells and the terminally differentiated cells [Potten, C. S. and Morris, R. J. (1988). Epithelial stem cells in vivo. J. Cell Sci. Suppl. 10, 45-62]. Thus, an adult tissue such as, for example, prostate tissue is digested with Collagenase and subjected to repeated unit gravity centrifugation to separate the epithelial structures of the prostate (e.g., organoids, acini and ducts) from the stromal cells. Organoids are then disaggregated into single cell suspensions by incubation with Trypsin/EDTA (Life Technologies, Paisley, UK) and the basal, CD44-positive, stem cells are isolated from the luminal, CD57-positive, terminally differentiated secretory cells, using anti-human CD44 antibody (clone G44-26; Pharmingen, Becton Dickinson, Oxford, UK) labeling and incubation with MACS (Miltenyi Biotec Ltd, Surrey, UK) goat anti-mouse IgG microbeads. The cell suspension is then applied to a MACS column and the basal cells are eluted and re-suspended in WAJC 404 complete medium [Robinson, E. J. et al. (1998). Basal cells are progenitors of luminal cells in primary cultures of differentiating human prostatic epithelium Prostate 37, 149-160].

Since basal stem cells can adhere to basement membrane proteins more rapidly than other basal cells [Jones, P. H. et al. (1995). Stem cell patterning and fate in human epidermis. Cell 60, 83-93; Shinohara, T., et al. (1999). β1- and α6-integrin are surface markers on mouse spermatogonial stem cells. Proc. Natl. Acad. Sci. USA 96, 5504-5509] the CD44 positive basal cells are plated onto tissue culture dishes coated with either type I collagen (52 μg/ml), type IV collagen (88 μg/ml) or laminin 1 (100 μg/ml; Biocoat®, Becton Dickinson) previously blocked with 0.3% bovine serum albumin (fraction V, Sigma-Aldrich, Poole, UK) in Dulbecco's phosphate buffered saline (PBS; Oxoid Ltd, Basingstoke, UK). Following 5 minutes, the tissue culture dishes are washed with PBS and adherent cells, containing the prostate tissue basal stem cells are harvested with trypsin-EDTA.

BM-Derived Stem Cell, Mesenchymal Stem Cells

Preferably, the stem cells utilized by some embodiments of the invention are BM-derived stem cells including hematopoietic, stromal or mesenchymal stem cells (Dominici, M et al., 2001. Bone marrow mesenchymal cells: biological properties and clinical applications. J. Biol. Regul. Homeost. Agents. 15: 28-37). BM-derived stem cells may be obtained from iliac crest, femora, tibiae, spine, rib or other medullar spaces.

Of the above described BM-derived stem cells, mesenchymal stem cells are the formative pluripotent blast cells, and as such are preferred for use with some embodiments of the invention. Mesenchymal stem cells give rise to one or more mesenchymal tissues (e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the BM far exceeds their abundance in other tissues and as such isolation from BM is presently preferred.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

Preferably, mesenchymal stem cell cultures are generated by diluting BM aspirates (usually 20 ml) with equal volumes of Hank's balanced salt solution (HBSS; GIBCO Laboratories, Grand Island, N.Y., USA) and layering the diluted cells over about 10 ml of a Ficoll column (Ficoll-Paque; Pharmacia, Piscataway, N.J., USA). Following 30 minutes of centrifugation at 2,500×g, the mononuclear cell layer is removed from the interface and suspended in HBSS. Cells are then centrifuged at 1,500×g for 15 minutes and resuspended in a complete medium (MEM, a medium without deoxyribonucleotides or ribonucleotides; GIBCO); 20% fetal calf serum (FCS) derived from a lot selected for rapid growth of MSCs (Atlanta Biologicals, Norcross, Ga.); 100 units/ml penicillin (GIBCO), 100 μg/ml streptomycin (GIBCO); and 2 mM L-glutamine (GIBCO). Resuspended cells are plated in about 25 ml of medium in a 10 cm culture dish (Corning Glass Works, Corning, N.Y.) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, PA). Cultured cells are recovered by centrifugation and resuspended with 5% DMSO and 30% FCS at a concentration of 1 to 2×10⁶ cells per ml. Aliquots of about 1 ml each are slowly frozen and stored in liquid nitrogen.

To expand the mesenchymal stem cell fraction, frozen cells are thawed at 37° C., diluted with a complete medium and recovered by centrifugation to remove the DMSO. Cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000 fold [Colter D C., et al. Rapid expansion of recycling stem cells in cultures of plastic-adherent cells from human bone marrow. Proc Natl Acad Sci USA. 97: 3213-3218, 2000].

MSC cultures utilized by some embodiments of the invention preferably include three groups of cells which are defined by their morphological features: small and agranular cells (referred to as RS-1, hereinbelow), small and granular cells (referred to as RS-2, hereinbelow) and large and moderately granular cells (referred to as mature MSCs, hereinbelow). The presence and concentration of such cells in culture can be assayed by identifying a presence or absence of various cell surface markers, by using, for example, immunofluorescence, in situ hybridization, and activity assays.

When MSCs are cultured under the culturing conditions of some embodiments of the invention they exhibit negative staining for the hematopoietic stem cell markers CD34, CD11B, CD43 and CD45. A small fraction of cells (less than 10%) are dimly positive for CD31 and/or CD38 markers. In addition, mature MSCs are dimly positive for the hematopoietic stem cell marker, CD117 (c-Kit), moderately positive for the osteogenic MSCs marker, Stro-1 [Simmons, P. J. & Torok-Storb, B. (1991). Blood 78, 5562] and positive for the thymocytes and peripheral T lymphocytes marker, CD90 (Thy-1). On the other hand, the RS-1 cells are negative for the CD117 and Stro1 markers and are dimly positive for the CD90 marker, and the RS-2 cells are negative for all of these markers.

Currently practiced ES culturing methods are mainly based on the use of feeder cell layers which secrete factors needed for stem cell proliferation, while at the same time, inhibit their differentiation. Feeder cell free systems have also been used in ES cell culturing, such systems utilize matrices supplemented with serum, cytokines and growth factors as a replacement for the feeder cell layer.

Media for culturing stem cells are known in the art. The growth medium can be supplemented with nutritional factors, such as amino acids, (e.g., L-glutamine), anti-oxidants (e.g., beta-mercaptoethanol) and growth factors, which benefit stem cell growth in an undifferentiated state. Serum and serum replacements may be added at effective concentration ranges as described elsewhere.

According to one embodiment, the stem cells are cultured such that they undergo at least one round of propagation, at least two rounds of propagation, at least 3 rounds of propagation, at least 4 rounds of propagation, at least 5 rounds of propagation, at least 6 rounds of propagation, at least 7 rounds of propagation, at least 8 rounds of propagation, at least 9 rounds of propagation or at least 10 rounds of propagation.

It will be appreciated that the outcome of propagating stem cells according to this aspect of the present invention does not alter the differentiation potential of the cells. Thus, for example propagation of pluripotent stem cells (e.g. embryonic stem cells) according to the disclosed method does not affect the pluripotency of the cells (i.e. they are still capable of differentiating into lineages of all three germ layers).

As mentioned, propagation of stem cells is achieved by up-regulating the expression level and/or activity a component participating in the mitochondrial apoptotic pathway in the subject (the pathway being as detailed herein above). Up-regulating the expression level and/or activity of a component participating in the mitochondrial apoptotic pathway can be achieved in any of various ways, which are further described herein below.

Using various conditional MTCH2 knock-out mice, the present inventors have now discovered that down-regulation of MTCH2 has a myriad of different effects on the physiology of the animal. Accordingly, the present inventors propose up- or down-regulation of MTCH2 for the treatment for a variety of different diseases in a subject in need thereof.

For example, the present inventors propose up-regulation of MTCH2 (or an agonist thereof) for the treatment of a hematological cancer, hyperactivity, retarded growth, learning and memory disability and male infertility; and down-regulation of MTCH2 (or an agonist thereof) for the treatment of radiation injury, enhancement of exercise endurance, enhancement of cardiac function and the treatment of cardiac disorders and the treatment of a fat-related disease.

The term “hematological cancer” herein includes a lymphoma, leukemia, myeloma or a lymphoid malignancy, as well as a cancer of the spleen and the lymph nodes. Exemplary lymphomas that are amenable to treatment with the disclosed agents include both B cell lymphomas and T cell lymphomas. B-cell lymphomas include both Hodgkin's lymphomas and most non-Hodgkins lymphomas. Non-limiting examples of B cell lymphomas include diffuse large B-cell lymphoma (DLBCL), follicular lymphoma (FL), mucosa-associated lymphatic tissue lymphoma (MALT), small cell lymphocytic lymphoma (overlaps with chronic lymphocytic leukemia), mantle cell lymphoma (MCL), Burkitt's lymphoma, mediastinal large B cell lymphoma, Waldenstrom macroglobulinemia, nodal marginal zone B cell lymphoma (NMZL), splenic marginal zone lymphoma (SMZL), intravascular large B-cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma. Hematological malignancies also include leukemia, such as, but not limited to, secondary leukemia, acute myelogenous leukemia (AML; also called acute lymphoid leukemia), chronic myelogenous leukemia (CML), B-cell prolymphocytic leukemia (B-PLL), acute lymphoblastic leukemia (ALL) and myelodysplasia (MDS). Hematological malignancies further include myelomas, such as, but not limited to, multiple myeloma (MM), smoldering multiple myeloma (SMM) and B-cell chronic lymphocytic leukemia (CLL).

According to a particular embodiment, the hematological malignancy is acute myeloid leukemia (AML).

Other hematological and/or B cell- or T-cell-associated cancers are encompassed by the term hematological malignancy. For example, hematological malignancies also include cancers of additional hematopoietic cells, including dendritic cells, platelets, erythrocytes, natural killer cells, and polymorphonuclear leukocytes, e.g., basophils, eosinophils, neutrophils and monocytes. It should be clear to those of skill in the art that these pre-malignancies and malignancies will often have different names due to changing systems of classification, and that patients having lymphomas classified under different names may also benefit from the therapeutic regimens of the present invention. Examples of fat-related diseases include but are not limited to obesity, fatty liver disease, heart disease, stroke, atherosclerosis, Diabetes, osteoarthritis, gout, sleep apnea, metabolic syndrome and high blood pressure.

According to one embodiment the subject has a body mass index (BMI) of greater than 30. Subjects having BMI between 25 and 30 are considered overweight and in one embodiment, are treated by the agents disclosed herein. The body mass index (BMI) is calculated by dividing an individual's weight in kilograms by the square of their height in meters. BMI does not distinguish fat mass from lean mass and an obese subject typically has excess adipose tissue.

Thus, in one embodiment of the present invention, the subject has a BMI greater than 30. In one embodiment, the subject has a BMI of 25 or over, e.g. 26, 27, 28, 29, 30, 31, 32, 33, 34, 35, 36, 37, 38, 39 or 40 or greater and has no obesity-related co-morbidity. In one embodiment, the patient is morbidly obese and has a BMI of 40 or over. In one embodiment, the subject is obese and/or suffering from complications associated with obesity. In one embodiment, the subject is obese and/or was suffering from complications associated with obesity, which have now been corrected. In one embodiment, the subject has a Body Mass Index (BMI) of over 25, and preferably over 30.

It will be appreciated that down-regulation of MTCH2 may also be a suitable therapy to promote weight loss in a non-obese (but overweight) subject.

According to a particular embodiment, the disease is not diabetes.

According to another embodiment, the disease is not obesity.

According to a particular embodiment, the fat-related disease is fatty liver disease.

As used herein, the term “fatty liver disease” refers to a disease or a pathological condition caused by, at least in part, abnormal hepatic lipid deposits. Fatty liver disease includes, e.g., alcoholic fatty liver disease, non-alcoholic fatty liver disease, and acute fatty liver of pregnancy. Fatty liver disease may be, e.g., macro-vesicular steatosis or micro-vesicular steatosis.

According to a particular embodiment, the disease is non-alcoholic fatty liver disease.

The non-alcoholic fatty liver disease may be a primary or a secondary non-alcoholic fatty liver disease.

The non-alcoholic fatty liver disease may be either familial or non-familial.

According to a particular embodiment, the familial fatty liver disease is familial hyperlipidemia.

Upregulation of Mtch2 can be advantageously used to treat disorders associated with, for example, necrotic, apoptotic, damaged, dysfunctional or morphologically abnormal myocardium. Such disorders include, but are not limited to, ischemic heart disease, cardiac infarction, rheumatic heart disease, endocarditis, autoimmune cardiac disease, valvular heart disease, congenital heart disorders, cardiac rhythm disorders, impaired myocardial conductivity and cardiac insufficiency.

According to one embodiment, the method according to this aspect of the present invention can be advantageously used to efficiently reverse, inhibit or prevent cardiac damage caused by ischemia resulting from myocardial infarction.

According to another embodiment, the method according to this aspect of the present invention can be used to treat cardiac disorders characterized by abnormal cardiac rhythm, such as, for example, cardiac arrhythmia.

As used herein the phrase “cardiac arrhythmia” refers to any variation from the normal rhythm of the heart beat, including, but not limited to, sinus arrhythmia, premature beat, heart block, atrial fibrillation, atrial flutter, pulsus alternans and paroxysmal tachycardia.

According to this aspect of the present invention, the term “subject” (or “individual” which is interchangeably used herein) refers to an animal subject e.g., a mammal, e.g., a human being at any age who suffers from or is at risk of developing the pathology. Non-limiting examples of individuals who are at risk to develop the pathology of the present invention include individuals who are genetically predisposed to develop the pathology (e.g., individuals who carry a mutation or a DNA polymorphism which is associated with high prevalence of the pathology), and/or individuals who are at high risk to develop the pathology due to other factors such as environmental hazard or other pathologies.

Decreasing the expression level of a component of the mitochondrial apoptotic pathway (e.g. MTCH2) can be effected on the genomic and/or the transcript level using a variety of molecules which interfere with transcription and/or translation (e.g., antisense, siRNA, Ribozyme, DNAzyme), or on the protein level using e.g., antagonists, enzymes that cleave the polypeptide and the like.

Following is a list of agents capable of decreasing the expression level of a component of the mitochondrial apoptotic pathway (e.g. MTCH2).

One example of an agent capable of downregulating (or decreasing the expression level of) a component of the mitochondrial apoptotic pathway (e.g. MTCH2) is an antibody or antibody fragment capable of specifically binding thereto. According to one embodiment, the antibody inhibits the activity of the protein. According to a particular embodiment, the antibody destabilizes the complex between MTCH2 and its agonist. Preferably, the antibody specifically binds at least one epitope of the component (e.g., MTCH2). As used herein, the term “epitope” refers to any antigenic determinant on an antigen to which the paratope of an antibody binds. Exemplary antibodies are disclosed in US Application No. 20090124564, incorporated herein by reference.

Epitopic determinants usually consist of chemically active surface groupings of molecules such as amino acids or carbohydrate side chains and usually have specific three dimensional structural characteristics, as well as specific charge characteristics.

For example, to downregulate the expression level of human MTCH2, the antibody can be directed against an epitope (e.g., a peptide of 3-8 amino acids) selected from the polypeptide set forth by SEQ ID NO:11.

Downregulation of a component of the mitochondrial apoptotic pathway can be also achieved using an agent capable of decreasing (i.e., downregulating) the tBID binding activity of MTCH2. Such an agent can be, for example, an antibody, a peptide or a small molecule capable of preventing the formation or destabilizing the MCD-containing protein-tBID complex.

It will be appreciated that such an antibody which is used to decrease the tBID-binding activity of MTCH2 or to destabilize the complex therebetween can be directed against MTCH2 itself, and preferably, against the tBID-binding domain on the MTCH2. Non-limiting examples of such an epitope are disclosed in US Application No. 20090124564, incorporated herein by reference. Additionally or alternatively, the antibody can be directed against the complex itself. It will be appreciated that such antibodies can be generated using methods known in the art by injecting a substantially pure preparation of the MTCH2-tBID complex into an immunizing animal.

The term “antibody” as used in this invention includes intact molecules as well as functional fragments thereof, such as Fab, F(ab′)2, Fv or single domain molecules such as VH and VL to an epitope of an antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; and (6) Single domain antibodies are composed of a single VH or VL domains which exhibit sufficient affinity to the antigen.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference and the Examples section which follows).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments.

Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

It will be appreciated that targeting of particular compartment within the cell can be achieved using intracellular antibodies (also known as “intrabodies”). These are essentially SCA to which intracellular localization signals have been added (e.g., ER, mitochondrial, nuclear, cytoplasmic). This technology has been successfully applied in the art (for review, see Richardson and Marasco, 1995, TIBTECH vol. 13). Intrabodies have been shown to virtually eliminate the expression of otherwise abundant cell surface receptors and to inhibit a protein function within a cell (See, for example, Richardson et al., 1995, Proc. Natl. Acad. Sci. USA 92: 3137-3141; Deshane et al., 1994, Gene Ther. 1: 332-337; Marasco et al., 1998 Human Gene Ther 9: 1627-42; Shaheen et al., 1996 J. Virol. 70: 3392-400; Werge, T. M. et al., 1990, FEBS Letters 274:193-198; Carlson, J. R. 1993 Proc. Natl. Acad. Sci. USA 90:7427-7428; Biocca, S. et al., 1994, Bio/Technology 12: 396-399; Chen, S-Y. et al., 1994, Human Gene Therapy 5:595-601; Duan, L et al., 1994, Proc. Natl. Acad. Sci. USA 91:5075-5079; Chen, S-Y. et al., 1994, Proc. Natl. Acad. Sci. USA 91:5932-5936; Beerli, R. R. et al., 1994, J. Biol. Chem. 269:23931-23936; Mhashilkar, A. M. et al., 1995, EMBO J. 14:1542-1551; PCT Publication No. WO 94/02610 by Marasco et al.; and PCT Publication No. WO 95/03832 by Duan et al.).

To prepare an intracellular antibody expression vector, the cDNA encoding the antibody light and heavy chains specific for the target protein of interest are isolated, typically from a hybridoma that secretes a monoclonal antibody specific for the marker.

Hybridomas secreting anti-marker monoclonal antibodies, or recombinant monoclonal antibodies, can be prepared using methods known in the art. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

For mitochondrial expression of the light and heavy chains, the nucleotide sequences encoding the mitochondrial targeting sequences are added [e.g., the COOH-terminal signal anchor of Bcl-2 (Nguyen, M. et al., 1993. J. Biol. Chem. 268:25265-25268)]. An intracellular antibody expression vector can encode an intracellular antibody in one of several different forms. For example, in one embodiment, the vector encodes full-length antibody light and heavy chains such that a full-length antibody is expressed intracellularly. In another embodiment, the vector encodes a full-length light chain but only the VH/CH1 region of the heavy chain such that a Fab fragment is expressed intracellularly. In another embodiment, the vector encodes a single chain antibody (scFv) wherein the variable regions of the light and heavy chains are linked by a flexible peptide linker and expressed as a single chain molecule. To inhibit marker activity in a cell, the expression vector encoding the intracellular antibody is introduced into the cell by standard transfection methods, as discussed hereinbefore.

Peptides used to decrease the tBID-binding activity of the MTCH2 or to destabilize the MTCH2-tBID complex can be any peptide (from e.g., 5 amino acids to 25 amino acids in length) having the affinity to both proteins at the site of interaction therebetween. Non-limiting examples of such peptides are disclosed in U.S. Patent Application No. 20090124564, incorporated herein by reference.

.Another agent capable of downregulating a component of the mitochondrial apoptotic pathway (e.g. MTCH2) is a small interfering RNA (siRNA) molecule. RNA interference is a two step process. In the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the mRNA sequence encoding the MCD-containing protein (e.g., Mtch2; SEQ ID NO:16; GenBank Accession No. NM_014342) is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl Chem Biochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level.

Second, potential target sites are compared to an appropriate genomic database (e.g., human, mouse, rat etc.) using any sequence alignment software, such as the BLAST software available from the NCBI server. Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

The selected siRNAs can be chemically synthesized oligonucleotides (using e.g., solid phase synthesis) or can be encoded from plasmids in order to induce RNAi in cells following transfection (using e.g., the pRETRO-SUPER vector as described in the Examples section which follows). Recently, retrovirus- or lentivirus-delivered RNAi were developed and were found efficient in long-term gene silencing in vivo [Hao DL., et al., 2005, Acta. Biochim. Biophys. Sin. (Shanghai), 37(11): 779-83].

For example, a suitable Mtch2 siRNA can be the siRNA set forth by SEQ ID NO: 14.

Another agent capable of downregulating a component of the mitochondrial apoptotic pathway (e.g. MTCH2) is a DNAzyme molecule capable of specifically cleaving an mRNA transcript or DNA sequence of the protein. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther). In another application, DNAzymes complementary to bcr-ab1 oncogenes were successful in inhibiting the oncogenes expression in leukemia cells, and lessening relapse rates in autologous bone marrow transplant in cases of CML and ALL.

Downregulation of a component of the mitochondrial apoptotic pathway (e.g. MTCH2) can also be effected by using an antisense polynucleotide capable of specifically hybridizing with an mRNA transcript encoding the component (e.g., MTCH2).

Design of antisense molecules which can be used to efficiently downregulate a component of the mitochondrial apoptotic pathway (e.g. MTCH2) must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries. In addition, several approaches for designing and predicting efficiency of specific oligonucleotides using an in vitro system were also published (Matveeva et al., Nature Biotechnology 16: 1374 - 1375 (1998)].

For example, a suitable antisense oligonucleotide targeted against the mRNA coding for MTCH2 would be of the following sequences: 5′-TCCTCACCCTTGTCACTCTCC-3′ [SEQ ID NO: 15; corresponds to nucleic acids 314-334 of SEQ ID NO: 16; designed using the IDT design tool according to the Matveeva rule set].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating the expression of a component of the mitochondrial apoptotic pathway (e.g. MTCH2) is a ribozyme molecule capable of specifically cleaving an mRNA transcript encoding same (e.g., MTCH2, SEQ ID NO: 16). Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Another agent which can be used to downregulate the expression level of a component of the mitochondrial apoptotic pathway (e.g. MTCH2) in cells is a triplex forming oligonucleotide (TFO). Recent studies have shown that TFOs can be designed which can recognize and bind to polypurine/polypirimidine regions in double-stranded helical DNA in a sequence-specific manner. These recognition rules are outlined by Maher III, L. J., et al., Science, 1989, 245:725-730; Moser, H. E., et al., Science, 1987, 238:645-630; Beal, P. A., et al, Science,1992, 251:1360-1363; Cooney, M., et al., Science, 1988, 241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer, J Clin Invest 2003; 112:487-94).

In general, the triplex-forming oligonucleotide has the sequence correspondence:

| | | | | |
|---|---|---|---|---|
| oligo | 3′-A | G | G | T |
| duplex | 5′-A | G | C | T |
| duplex | 3′-T | C | G | A |

However, it has been shown that the A-AT and G-GC triplets have the greatest triple helical stability (Reither and Jeltsch, BMC Biochem, 2002, 3: 27.). The same authors have demonstrated that TFOs designed according to the A-AT and G-GC rule do not form non-specific triplexes, indicating that the triplex formation is indeed sequence specific.

Thus for any given sequence in the component of the mitochondrial apoptotic pathway (e.g. MTCH2), a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. 1999; 27:1176-81, and Puri, et al, J Biol Chem, 2001; 276:28991-98), and the sequence- and target specific downregulation of expression of the Ets2 transcription factor, important in prostate cancer etiology (Carbone, et al, Nucl Acid Res. 2003; 31:833-43), and the pro-inflammatory ICAM-1 gene (Besch et al, J Biol Chem, 2002; 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res 2000; 28:2369-74).

Additionally, TFOs designed according to the abovementioned principles can induce directed mutagenesis capable of effecting DNA repair, thus providing both downregulation and upregulation of expression of endogenous genes (Seidman and Glazer, J Clin Invest 2003;112:487-94). Detailed description of the design, synthesis and administration of effective TFOs can be found in U.S. Patent Application Nos. 2003 017068 and 2003 0096980 to Froehler et al., and 2002 0128218 and 2002 0123476 to Emanuele et al., and U.S. Pat. No. 5,721,138 to Lawn.

As mentioned, for treatment of particular diseases and disorders, up-regulation of a component of the mitochondrial apoptotic pathway is required. Agents capable of upregulating one of these components include for example a peptide, an antibody (e.g., a stabilizing antibody) or a small molecule which, when added to cells expressing the MTCH2-tBID complex, is capable of stabilizing (i.e., prolonging the half-life) of such a complex.

The term “peptide” as used herein encompasses synthetic or naturally occurring peptides, peptide analogues or mimetics thereof. The term “peptide” preferably refers to short amino acid sequences of at least 2 or 3, preferably at least 4, more preferably, at least 5, more preferably, in the range of 5-30, even more preferably in the range of 5-25 natural or non-natural amino acids which are capable of the biological activity (i.e., stabilizing the MTCH2-tBID complex in this case).

As used herein the term “mimetics” refers to molecular structures, which serve as substitutes for the peptide of the present invention in performing the biological activity (Morgan et al. (1989) Ann. Reports Med. Chem. 24:243-252 for a review of peptide mimetics). Peptide mimetics, as used herein, include synthetic structures (known and yet unknown), which may or may not contain amino acids and/or peptide bonds, but retain the structural and functional features of the peptide. Types of amino acids which can be utilized to generate mimetics are further described hereinbelow. The term, “peptide mimetics” also includes peptoids and oligopeptoids, which are peptides or oligomers of N-substituted amino acids [Simon et al. (1972) Proc. Natl. Acad. Sci. USA 89:9367-9371]. Further included as peptide mimetics are peptide libraries, which are collections of peptides designed to be of a given amino acid length and representing all conceivable sequences of amino acids corresponding thereto. Methods of producing peptide mimetics are described hereinbelow.

The peptides of present invention can be biochemically synthesized such as by using standard solid phase techniques. These methods include exclusive solid phase synthesis, partial solid phase synthesis methods, fragment condensation and classical solution synthesis. These methods are preferably used when the peptide is relatively short (i.e., 10 kDa) and/or when it cannot be produced by recombinant techniques (i.e., not encoded by a nucleic acid sequence) and therefore involve different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680, Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

Upregulation of the expression level of a component of the mitochondrial apoptotic pathway can be effected at the genomic level (i.e., activation of transcription via promoters, enhancers, regulatory elements), at the transcript level (i.e., correct splicing, polyadenylation, activation of translation) or at the protein level (i.e., post-translational modifications).

Preferably, upregulation of the expression level of a component of the apoptotic pathway is effected by contacting the cell with an exogenous polypeptide including at least a functional portion of the component.

As used herein, the phrase “polypeptide” encompasses a naturally occurring polypeptide which is comprised solely of natural amino acid residues or synthetically prepared polypeptides, comprised of a mixture of natural and modified (non-natural) amino acid residues as described hereinabove.

The phrase “functional portion” as used herein refers to at least a portion of the polypeptide of the present invention which is sufficient to downregulate apoptosis.

It will be appreciated that for large polypeptides (e.g., above 25 amino acids), the exogenous polypeptide is preferably prepared using recombinant techniques.

For example, to generate the protein which is part of the pathway a polypeptide such as human MTCH2; SEQ ID NO:11), a polynucleotide sequence encoding the protein (e.g., GenBank Accession number NM_014342; SEQ ID NO:16) or a functional portion thereof is preferably ligated into a nucleic acid construct suitable for expression in a host cell. Such a nucleic acid construct includes a promoter sequence for directing transcription of the polynucleotide sequence in the cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter [Zabala M, et al., Cancer Res. 2004, 64(8): 2799-804].

The nucleic acid construct (also referred to herein as an “expression vector”) of the present invention includes additional sequences which render this vector suitable for replication and integration in prokaryotes, eukaryotes, or preferably both (e.g., shuttle vectors). In addition, a typical expression vector may also contain a transcription and translation initiation sequence, enhancers (e.g., SV40 early gene enhancer; see also Enhancers and Eukaryotic Expression, Cold Spring Harbor Press, Cold Spring Harbor, N.Y. 1983), transcription and translation terminator, and a polyadenylation signal which may increase the efficiency of mRNA translation (e.g., the GU or U rich sequences located downstream from the polyadenylation site and a highly conserved sequence of six nucleotides, AAUAAA, located 11-30 nucleotides upstream). It will be appreciated that in order to secret the recombinant polypeptide from the host cell (i.e., a cell in which the polynucleotide of the present invention is expressed) the expression vector of the present invention typically includes a signal sequence for secretion.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Other than containing the necessary elements for the transcription and translation of the inserted coding sequence, the expression construct of the present invention can also include sequences engineered to enhance stability, production, purification, yield or toxicity of the expressed polypeptide.

As mentioned hereinabove, a variety of cells can be used as host-expression systems to express the recombinant polypeptide of the present invention (e.g., human Mtch2). These include, but are not limited to, microorganisms, such as bacteria transformed with a recombinant bacteriophage DNA, plasmid DNA or cosmid DNA expression vector containing the polypeptide coding sequence, mammalian expression systems, yeast transformed with recombinant yeast expression vectors containing the coding sequence (see for example, U.S. Pat. No. 5,932,447); plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence [for suitable plant expression vectors see for example, Brisson et al. (1984) Nature 310:511-514; Takamatsu et al. (1987) EMBO J. 6:307-311; Coruzzi et al. (1984) EMBO J. 3:1671-1680; Brogli et al., (1984) Science 224:838-843; Gurley et al. (1986) Mol. Cell. Biol. 6:559-565; Weissbach & Weissbach, 1988, Methods for Plant Molecular Biology, Academic Press, NY, Section VIII, pp 421-463]. Bacterial systems are preferably used to produce recombinant polypeptides since they enable a high production volume at low cost.

In bacterial systems, a number of expression vectors can be advantageously selected depending upon the use intended for the polypeptide expressed. For example, when large quantities of polypeptide are desired, vectors that direct the expression of high levels of the protein product, possibly as a fusion with a hydrophobic signal sequence, which directs the expressed product into the periplasm of the bacteria or the culture medium where the protein product is readily purified may be desired. Certain fusion protein engineered with a specific cleavage site to aid in recovery of the polypeptide may also be desirable. Such vectors adaptable to such manipulation include, but are not limited to, the pET series of E. coli expression vectors [Studier et al., Methods in Enzymol. 185:60-89 (1990)].

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Stratagene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMT010/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the expression vector of the present invention into host cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency and specificity can be obtained due to the infectious nature of viruses.

Transformed cells are cultured under effective conditions, which allow for the expression of high amounts of the recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the cell, secreted into the fermentation medium, secreted into a space between two cellular membranes, such as the periplasmic space in E. coli; or retained on the outer surface of a cell or viral membrane.

Following a predetermined time in culture, recovery of the recombinant polypeptide is effected. The phrase “recovery of the recombinant polypeptide” used herein refers to collecting the whole fermentation medium containing the polypeptide and need not imply additional steps of separation or purification.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

To facilitate recovery, the expressed coding sequence can be engineered to encode the polypeptide of the present invention and a fused cleavable moiety. Such a fusion protein can be designed so that the polypeptide can be readily isolated by affinity chromatography; e.g., by immobilization on a column specific for the cleavable moiety. Where a cleavage site is engineered between the polypeptide and the cleavable moiety, the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

The polypeptide of the present invention is preferably retrieved in “substantially pure” form. As used herein, the phrase “substantially pure” refers to a purity that allows for the effective use of the recombinant polypeptide in the methods described herein.

Another agent capable of upregulating the expression level of a component of the mitochondrial apoptotic pathway is an exogenous polynucleotide sequence designed and constructed to express in cells at least a functional portion of the protein. Accordingly, the exogenous polynucleotide sequence may be a DNA or RNA sequence encoding the component of the pathway.

It will be appreciated that for ex vivo or in vivo gene therapy applications which are further described hereinunder the exogenous polynucleotide of the present invention is administered to the cell-of-interest so as to be expressed. As used herein, the phrase “ex vivo gene therapy” refers to the process of expressing the polypeptide of the present invention in cell cultures derived from a subject (e.g., autologous or allogeneic cells) followed by administration of such cells (which express the recombinant polypeptide of the present invention) back into the subject in need of therapy. The phrase “in vivo gene therapy” refers to the process of expressing the polypeptide of the present invention in cells of the subject in need of therapy.

It will be appreciated that the type of viral vector and the specific promoter used for ex vivo or in vivo gene therapy will depend on the cell type transformed. The ability to select suitable vectors according to the cell type transformed is well within the capabilities of the ordinary skilled artisan and as such no general description of selection consideration is provided herein.

Recombinant viral vectors are useful for in vivo expression of recombinant proteins since they offer advantages such as lateral infection and targeting specificity.

Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.It will be appreciated that various methods can be used to qualify the ability of the agents of the present invention to upregulate or downregulate a component of the mitochondrial apoptotic pathway as described in US Patent Application No. 20090124564.

It will be appreciated that the agent of the present invention (e.g., the antibody, the siRNA, the exogenous polypeptide, the exogenous polynucleotide or the expression vector encoding same) can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent of the present invention (e.g., the antibody, the siRNA, the exogenous polypeptide, the exogenous polynucleotide or the expression vector encoding same) which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, neurosurgical strategies (e.g., intracerebral injection, intrastriatal infusion or intracerebroventricular infusion, intra spinal cord, epidural), transmucosal, intestinal or parenteral delivery, including intramuscular, subcutaneous and intramedullary injections as well as intrathecal, direct intraventricular, intracardiac, intravenous, intraperitoneal, intranasal, or intraocular injections.

Alternately, one may administer the pharmaceutical composition in a local rather than a systemic manner, for example, via injection of the pharmaceutical composition directly into a tissue region of a patient.

Intra-brain infusions can be performed using an implanted device as described in Sanftner L M, et al., 2005 (Exp Neurol. 194(2):476-83) for administration of recombinant adeno-associated virus (AAV2) to the brain.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes.

Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose (i.e., a therapeutically effective amount as described hereinabove).

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide tissue levels of the active ingredient that are sufficient to regulate apoptosis (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### The Role of Mtch2 in Hematopoietic Stem Cells

Materials and Methods

Mice: MTCH2^{F/F} mice were generated as described¹⁶. Vav1-Cre mice (on a C57BL/6 background) were purchased from Jackson Laboratory. C57BL/6 CD45.1 congenic mice were from the Weizmann Institute of Science and C57BL/6 CD45.2 congenic mice were purchased from Harlan, Rehovot, Israel. TBI experiments were performed as described¹⁴. All data presented were repeated in at least three independent experiments with 8- to 10-week-old littermate mice.

Flow cytometry: For flow cytometric analyses the following monoclonal antibodies were used: c-Kit (105812; clone 2B8), Sca-1 (108116; clone D7), CD150 (115903; clone TC15-12F12.2), CD48 (103409; clone HM48-1), CD41 (13-0411; clone MWReg30), B220 (103204; clone RA3-6B2), CD115 (135506; cloneAFS98), CD11b (101204; clone M1-70), Gr-1 (108406; clone RB6-8C5), Ter119 (116204; clone TER-119), CD3 (100304; clone 145- 2C11) and CD45.1 (110704; clone A20), IL7R (135007; clone A7R34), FcγR (139303; clone X54-5/7.1). All the antibodies were purchased from BioLegend, except for antibodies to CD41 (13-0411; clone MWReg30) and B220 (17-0552-81; clone RA3-6B2), which were purchased from eBioscience. All the antibodies were used at a dilution of 1:100. Streptavidin PE/Cy7 (405206) was purchased from BioLegend and used at a dilution of 1:200. All analyses were done using an LSRII flow cytometer (BD Bioscience); data analysis was done using the FlowJo software.

Cell cycle: To analyze quiescence of cells, total bone-marrow cells stained for the designated markers were fixed, permeabilized using the Cytofix/Cytoperm kit (BD Bioscience) and stained with the Ki-67 antibody (BD Bioscience) and DAPI (4′,6-Diamidino-2-phenylindole dihydrochloride; 5 μg ml⁻¹; Sigma). To determine the proliferation status of cells, mice were given one daily dose of BrdU (5-Bromo-2-DeoxyUridine; 3.3 mg per mouse) by i.p. injections for three consecutive days, and mice were killed for analysis 2 h following the last administration. BrdU incorporation was assessed using the BrdU Flow Kit (BD Bioscience), according to the manufacturers' instructions.

Colony-forming assay: For colony-formation assays, 100,000 bone-marrow mono nuclear cells were plated in methyl cellulose (R&D Systems) in the presence of 10 ng ml⁻¹ of the following growth factors: IL-3, IL-6, SCF, GM-CSF, M-CSF, G-CSF (Peprotech). Colony number (CFU-C) was enumerated following 7 days in culture.

Competitive bone marrow transplantations: Competitive bone marrow transplantations were done as previously described¹⁴.

Quantitative real-time PCR: RT-PCR was done as previously described¹⁴. Primer sequences used for all the genes tested are listed below:

| SEQ ID NO: 1 | |
|---|---|
| MTCH2 | F-TGTTCACAGGCTTGACTCCA |
| SEQ ID NO: 2 | |
| MTCH2 | R-CAAACTGTATAGGTGAATGGCTCT |
| SEQ ID NO: 3 | |
| Ndufs8 | F-GACTGGGCATGACCCTAAGTT |
| SEQ ID NO: 4 | |
| Ndufs8 | R-CGCTCCTCTCCAGATGGGTA |
| SEQ ID NO: 5 | |
| SDHb | F-AATTTGCCATTTACCGATGGGA |
| SEQ ID NO: 6 | |
| SDHb | R-AGCATCCAACACCATAGGTCC |
| SEQ ID NO: 7 | |
| Cox5b | F-ACCCTAATCTAGTCCCGTCC |
| SEQ ID NO: 8 | |
| Cox5b | R-CAGCCAAAACCAGATGACAG |
| SEQ ID NO: 9 | |
| ATP5a1 | F-CATTGGTGATGGTATTGCGC |
| SEQ ID NO: 10 | |
| ATP5a1 | R-TCCCAAACACGACAACTCC |

Respiration: Freshly isolated total bone-marrow cells were enriched for CD117 (c-kit) population using Magnetic cell sorting (MACS; Miltenyi Biotec 130-091-224) and 0.5×10⁶ cells were immobilized to XF 24 plate (Seahorse Bioscience, Cat. #100777-004) pretreated with Cell-Tak™ (Corning; Cat #354240). Measurement of intact cellular respiration was performed using the Seahorse XF24 analyzer (Seahorse Bioscience Inc.) and the XF Cell Mito Stress Test Kit according to manufacturer's instruction. Respiration was measured under basal conditions, and in response to Oligomycin (ATP Coupler; 0.5 μM) and the electron transport chain accelerator ionophore FCCP (Trifluorocarbonylcyanide Phenylhydrazone; 0.5 μM). FCCP treatment gives two indexes: the Maximal OCR (Oxygen Consumption Rate) capacity of the cells and their spare respiratory capacity (indicated by maximum OCR calculated as percentage of baseline OCR). Finally, respiration was stopped by adding electron transport chain inhibitors Rotenone and Antimycin A (1 μM each).

NADH: NADH autofluorescence was measured flow cytometrically after excitation with a UV laser with a main emission peak at 470 nm. Mitochondrial NADH levels were calculated as the difference in arbitrary units between the maximum NADH autofluorescence in response to 1 mM KCN (Sigma) and minimum NADH autofluorescence in response to 1 μM (FCCP; Seahorse Bioscience Inc). NADH redox index was estimated by calculating the initial NADH autofluorescence when the minimum NADH autofluorescence is normalized to 0% and the maximum to 100%.

ROS: ROS levels (superoxide anion) were assessed by 20 min incubation at 37° C. of bone-marrow cells with 5 μM hydroethidine (DHE; Molecular Probes) for cellular ROS or 10 μM MitoSOX (Molecular Probes) for mitochondrial ROS. Cells were then washed once with PBS and stained for HSC markers. Fluorescence of oxidized DHE or MitoSOX was determined flow cytometrically.

Mitochondrial membrane potential: Total bone marrow cells were incubated with 50 nm Tetramethylrhodamine ethyl ester perchlorate (TMRE; Enzo Life Sciences) for 20 min at 37° C., washed once with PBS and stained for HSC markers. Fluorescence of TMRE was determined flow cytometrically. For mitochondrial mass estimation, 100 nm Mito Tracker Green (MTG; Molecular Probes) was added together with TMRE.

Transmission electron microscopy: Cells were fixed with 3% paraformaldehyde, 2% glutaraldehyde in 0.1 M cacodylate buffer containing 5 mM CaCl₂ (pH 7.4), than postfixed in 1% osmium tetroxide supplemented with 0.5% potassium hexacyanoferrate tryhidrate and potassium dichromate in 0.1 M cacodylate (1 hour), stained with 2% uranyl acetate in water (1 hour), dehydrated in graded ethanol solutions and embedded in Agar 100 epoxy resin (Agar scientific Ltd., Stansted, UK). Ultrathin sections (70-90 nm) were viewed and photographed with a FEI Tecnai SPIRIT (FEI, Eidhoven, Netherlands) transmission electron microscope operated at 120 kV and equipped with an EAGLE CCD Camera.

Caspase-3 activity: Mice were either left untreated or subjected to TBI and 0.5 h later total bone-marrow cells were enriched for CD117 (c-kit) population using Magnetic cell sorting (MACS; Miltenyi Biotec). Enriched cells were lysed with Promega cell lysis buffer (E1531) and caspase activity was estimated using Caspase-Glo® 3/7 Luminescent Assay kit (Promega) according to manufacturer's instructions.

ATP: Mice were either left untreated or subjected to TBI and 0.5 h later total bone-marrow cells were enriched for CD117 (c-kit) population using Magnetic cell sorting (MACS; Miltenyi Biotec). Enriched cells were lysed with Promega cell lysis buffer (E1531) and ATP amounts were done using ATPlite Luminescent kit (PerkinElmer) according to manufacturer's instructions.

Western blot: Western blot analysis was performed as previously described¹⁶. Antibodies used for western blotting included anti-cleaved caspase-3 (Cleaved Caspase-3 (Asp175) Antibody 9661s; Cell Signaling) and anti-βActin (A1978; Sigma).

Results

HSCs are mostly retained in a quiescent non-motile state in their bone marrow niches and shift to a migratory cycling and differentiating state, replenishing the blood with mature leukocytes and red blood cells upon demand. It was previously demonstrated that the ataxia-telangiectasia mutated (ATM) kinase plays an important role in regulating the self-renewal and quiescence of HSCs by regulating ROS levels^{12,13}. More recently the present inventors demonstrated that the BH3-only BID protein acts as a downstream ATM-effector in this pathway and regulates the quiescence of HSCs by regulating ROS levels produced by mitochondria¹⁴. However the exact mechanism by which BID regulates mitochondrial ROS and its relation to the switch to mitochondrial OxMetab described in the transition from stem to progenitor cell remained unknown¹⁵.

To address this issue, the present inventors analyzed mitochondrial carrier homolog 2 (MTCH2), BID's receptor-like protein in the mitochondria¹⁶. To determine whether MTCH2 is critical for HSC maintenance, they generated a genetic mouse model where MTCH2 deletion is driven by the Vav1-Cre allele¹⁷(MTCH2^{F/F} Vav1-cre⁺), resulting in haematopoietic-specific MTCH2 knockout (FIG. 1A). Upon MTCH2 deletion, a significant reduction in the HSC population in the bone marrow (CD150⁺CD48⁻CD41⁻LSK) was observed, associated with increased frequency of the multi-potent progenitors (CD150⁻CD48⁻CD41⁻LSK; MPPs) and common lymphoid progenitors (IL7R⁺ of Lin Sca-1⁻c-Kit⁺; CLP) (FIG. 1B), indicating enhanced lymphoid biased differentiation of HSCs. Analyzing the cell cycle status of MTCH2^{F/F} Vav1-cre⁺ HSCs and progenitor populations, the present inventors observed a significant reduction in quiescent HSCs (FIG. 1C), which was accompanied by enhanced proliferation of both HSCs and committed progenitors (FIG. 1D). The observed HSC exhaustion in MTCH2^{F/F} Vav1-cre⁺ mice resulted in reduced colony forming ability (FIG. 1E), a competitive disadvantage in bone-marrow repopulation (FIG. 1F) and mobilization/egress to peripheral blood (FIG. 1G). These results identify MTCH2 as a critical mitochondrial regulator of HSC quiescence and a potential downstream target of ATM and BID.

Next the present inventors analyzed the effect of MTCH2 knockout on mitochondrial function in haematopoietic stem/progenitor cells (HSPCs). They initially measured cellular respiration and revealed a significant increase in basal oxygen consumption and maximal respiration in MTCH2^{F/F} Vavl-cre⁺ HSPCs (FIG. 2A, left and middle panels). Importantly, the MTCH2 knockout HSPCs also possess substantial mitochondrial Spare Respiratory Capacity (SRC; FIG. 2A, right panel), which is the extra capacity available in cells to produce energy in response to increased stress and as such is associated with cellular survival^{18,19}. The increased respiration was accompanied by an expected decrease in mitochondrial NADH levels (FIG. 2B), due to increased consumption by the electron transport chain. Further characterization of the mitochondria in MTCH2^{F/F} Vav1-cre⁺ HSPCs showed an increase in the expression of nuclear-encoded subunits of the respiratory complexes (FIG. 2C), an increase in mitochondria membrane potential (ΔΨₘ; FIG. 2D), and a moderate increase in both cellular and mitochondrial ROS levels (FIG. 2E). Most interestingly, electron microscope analyses revealed that MTCH2^{F/F} Vav1-cre⁺ HSPC mitochondria were significantly enlarged (FIG. 2F), most likely to accommodate and allow the increase in OxMetab, as previously described²⁰.

It is well established that physiological enlargement of mitochondria, which correlates with improved mitochondrial ATP production, protects cells from stress-induced apoptosis²¹⁻²³. Since the ATM-BID couple is an important regulator of the DNA damage response in the bone marrow^{12,14,24} the present inventors tested the sensitivity of the MTCH2^{F/F} Vav1-cre⁺ mice to total body irradiation (TBI). Strikingly, it was found that loss of MTCH2 provides complete protection to HSPCs from irradiation-induced death in vivo (FIG. 3A), and that this protection was also evident in vitro (FIG. 3B). In accordance with these findings, the MTCH2^{F/F} Vav1-cre⁺ HSCPs were totally resistant to TBI-induced caspase-3 activation/cleavage in vivo (FIGS. 3C and 3D, respectively). Moreover, MTCH2-deficient HSPCs possess considerably higher levels of ATP under basal conditions (FIG. 3E), consistent with the idea that enlarged mitochondria and improved ATP production protects cells from stress-induced apoptosis.

The finding that mice lacking the MTCH2 protein in bone marrow exhibit increased mitochondrial oxidative phosphorylation (OXPHOS) that is sufficient to trigger haematopoietic stem cells (HSC) exit from quiescence suggested that MTCH2 may be involved with leukemia, especially in light of the fact that it was previously demonstrated that a subset of acute myeloid leukemia (AML) cells and patient samples, originating from the myeloid lineage of HSCs, have increased reliance on OXPHOS.

Bone marrow progenitor cells derived from MTCH2^{F/F} mice and MTCH2^{F/F} Vav1-cre+mice were infected with retrovirus containing MLL-AF9 (GFP) oncogene, known to induce AML, to create pre-leukemic cell lines. Prior to transplantation host wild type mice were sub-lethally irradiated (600 cGy) to ensure cells engraftment. The transplantation was performed by periocular injection of 1×10⁶ pre-leukemic cells.

As illustrated in FIGS. 3G-I, GFP positive cells are detected in the blood stream (G) and in the bone marrow (H) and spleen (I) one and two months after mice were transplanted with pre-leukemic bone marrow progenitor cells derived from the MTCH2 knock-out mice.

Furthermore, as illustrated in FIG. 3J, mice transplanted with pre-leukemic bone marrow progenitor cells derived from MTCH2 knock-out mice showed a lower survival rate than mice transplanted with pre-leukemic bone marrow progenitor cells derived from wild-type mice.

In summary, this study shows that mitochondrial OxMetab plays a critical role in determining the fate of HSCs (FIG. 3F). The present results demonstrate that loss of MTCH2 primes mitochondrial OxMetab, which acts as a catalyzer to drive HSCs into cycle. The robust increase in mitochondrial size and function results in an increase in both ATP and ROS levels that signal the dormant HSCs to cycle and concurrently equip them with enough energy to survive increased stress. Thus, a novel player was identified, that via tuning of mitochondrial OxMetab, dictates whether a stem cell will remain quiescent in the bone marrow niche or shift to a migratory cycling and differentiating state replenishing the blood with mature leukocytes.

### REFERENCES FOR EXAMPLE 1

1 Ito, K. & Suda, T. Metabolic requirements for the maintenance of self-renewing stem cells. Nature reviews. Molecular cell biology 15, 243-256, doi:10.1038/nrm3772 (2014).
2 Burgess, R. J., Agathocleous, M. & Morrison, S. J. Metabolic regulation of stem cell function. Journal of internal medicine, doi:10.1111/joim.12247 (2014).
3 Piccoli, C. et al. To breathe or not to breathe: the haematopoietic stem/progenitor cells dilemma. British journal of pharmacology 169, 1652-1671, doi:10.1111/bph.12253 (2013).
4 Takubo, K. et al. Regulation of the HIF-lalpha level is essential for hematopoietic stem cells. Cell stem cell 7, 391-402, doi:10.1016/j.stem.2010.06.020 (2010).
5 Takubo, K. et al. Regulation of glycolysis by Pdk functions as a metabolic checkpoint for cell cycle quiescence in hematopoietic stem cells. Cell stem cell 12, 49-61, doi:10.1016/j.stem.2012.10.011 (2013).
6 Yu, W. M. et al. Metabolic regulation by the mitochondrial phosphatase PTPMT1 is required for hematopoietic stem cell differentiation. Cell stem cell 12, 62-74, doi:10.1016/j.stem.2012.11.022 (2013).
7 Simsek, T. et al. The distinct metabolic profile of hematopoietic stem cells reflects their location in a hypoxic niche. Cell stem cell 7, 380-390, doi:10.1016/j.stem.2010.07.011 (2010).
8 Chen, C. et al. TSC-mTOR maintains quiescence and function of hematopoietic stem cells by repressing mitochondrial biogenesis and reactive oxygen species. The Journal of experimental medicine 205, 2397-2408, doi:10.1084/jem.20081297 (2008).
9 Ito, K. et al. A PML-PPAR-delta pathway for fatty acid oxidation regulates hematopoietic stem cell maintenance. Nature medicine 18, 1350-1358, doi:10.1038/nm.2882 (2012).
10 Orford, K. W. & Scadden, D. T. Deconstructing stem cell self-renewal: genetic insights into cell-cycle regulation. Nat Rev Genet 9, 115-128 (2008).
11 Signer, R. A. & Morrison, S. J. Mechanisms that regulate stem cell aging and life span. Cell stem cell 12, 152-165, doi:10.1016/j.stem.2013.01.001 (2013).
12 Ito, K. et al. Regulation of oxidative stress by ATM is required for self-renewal of haematopoietic stem cells. Nature 431, 997-1002 (2004).
13 Ito, K. et al. Reactive oxygen species act through p38 MAPK to limit the lifespan of hematopoietic stem cells. Nature medicine 12, 446-451 (2006).
14 Maryanovich, M. et al. The ATM-BID pathway regulates quiescence and survival of haematopoietic stem cells. Nature cell biology 14, 535-541, doi:10.1038/ncb2468 (2012).
15 Maryanovich, M. & Gross, A. A ROS rheostat for cell fate regulation. Trends Cell Biol 23, 129-134, doi:10.1016/j.tcb.2012.09.007 (2013).
16 Zaltsman, Y. et al. MTCH2/MIMP is a major facilitator of tBID recruitment to mitochondria. Nature cell biology 12, 553-562 (2010).
17 de Boer, J. et al. Transgenic mice with hematopoietic and lymphoid specific expression of Cre. European journal of immunology 33, 314-325 (2003).
18 Choi, S. W., Gerencser, A. A. & Nicholls, D. G. Bioenergetic analysis of isolated cerebrocortical nerve terminals on a microgram scale: spare respiratory capacity and stochastic mitochondrial failure. Journal of neurochemistry 109, 1179-1191, doi:10.1111/j.1471-4159.2009.06055.x (2009).
19 van der Windt, G. J. et al. Mitochondrial respiratory capacity is a critical regulator of CD8+ T cell memory development. Immunity 36, 68-78, doi:10.1016/j.immuni.2011.12.007 (2012).
20 Rossignol, R. et al. Energy substrate modulates mitochondrial structure and oxidative capacity in cancer cells. Cancer research 64, 985-993 (2004).
21 Blackstone, C. & Chang, C. R. Mitochondria unite to survive. Nature cell biology 13, 521-522, doi:10.1038/ncb0511-521 (2011).
22 Gomes, L. C., Di Benedetto, G. & Scorrano, L. During autophagy mitochondria elongate, are spared from degradation and sustain cell viability. Nature cell biology 13, 589-598, doi:10.1038/ncb2220 (2011).
23 Tondera, D. et al. SLP-2 is required for stress-induced mitochondrial hyperfusion. EMBO J 28, 1589-1600, doi:10.1038/emboj.2009.89 (2009).
24 Shiloh, Y. & Ziv, Y. The ATM protein kinase: regulating the cellular response to genotoxic stress, and more. Nature reviews. Molecular cell biology 14, 197-210 (2013).
25 Sahin, E. et al. Telomere dysfunction induces metabolic and mitochondrial compromise. Nature 470, 359-365, doi:10.1038/nature09787 (2011).
26 Plun-Favreau, H. et al. HtrA2 deficiency causes mitochondrial uncoupling through the F(1)F(0)-ATP synthase and consequent ATP depletion. Cell death & disease 3, e335, doi:10.1038/cddis.2012.77 (2012).

### Example 2

### The Role of Mtch2 in Skeletal Muscle

Generating a MTCH2 Muscle-Conditional Mouse

To explore the role of MTCH in skeletal muscle in vivo we mated the MTCH2 f/f mice with mice carrying a transgene for Cre-recombinase expressed under control of the muscle creatine kinase (MCK) promoter. The MCK promoter is expressed only in skeletal muscle and in heart. Expression of MCK begins at embryonic day 17 in the mouse, increases to ˜40% of maximal levels at birth, reaches maximal levels at day 10, and remains at a constantly high level throughout the rest of life. Thus MTCH2 knockout in the muscles of these mice will occur only after the muscle fibers are formed. Western blot analysis of several tissues prepared from the MTCH2 f/f and MTCH2 f/f MCK (MKO) mice demonstrated the absence of MTCH2 from MTCH2 MKO skeletal muscle and heart tissues, whereas the levels of MTCH2 in the white fat and brain tissues were normal (FIG. 4). These results indicate specific deletion of MTCH2 in the skeletal muscle and heart in the MKO mice.

MKO Mice Eat More

To characterize the effect of knocking out MTCH2 in the muscle the present inventors monitored the metabolic behavior of MKO mice using metabolic cages (TSE system). Metabolic cages are used to perform indirect calorimetry and simultaneously measure food/water intake, energy expenditure, and physical activity in conscious mice. The experiment was performed on 12-14 weeks adult male MKO mice and their f/f control mice (WT). FIGS. 5A and 5B show that on Normal Chow Diet (NCD) MKO mice eat and drink more than their corresponding control mice. However, their body weight remains similar to the body weight of control mice (data not shown).

MKO Mice Gain Less Weight on HFD

Next the effect of metabolic stressed diet was explored. MKO mice and their corresponding control littermates were challenged with high fat diet (HFD). Two additional diets served as controls: low fat diet (LFD), as a calories control diet, and NCD. MKO (n=12) and WT (n=11) mice were fed on a HFD (45% fat energy) for 13 weeks. In parallel, cohort of MKO (n=12) and WT (n=9) mice was fed on low fat diet (LFD; 10% fat energy) and NCD (n=8). See Table 1, herein below for diet composition. WT mice fed on HFD for 13 weeks gained significantly more weight compared to WT mice fed on LFD (FIG. 5A). In contrast, MKO mice did not gain significantly more weight when fed on HFD compared to LFD (FIG. 5A). At the end of the experiment whole body composition was determined by use of the nuclear magnetic resonance imaging (MRI) technology produced by Echo Medical Systems LTD (Houston, Tex.), and as expected the % fat of the MKO mice on HFD was significantly lower than in WT mice (FIG. 5B).

TABLE 1

| HFD | LFD | NCD | |
|---|---|---|---|
| 20 | 20 | 25 | Protein |
| 35 | 70 | 58 | Carbohydrate |
| 45 | 10 | 17 | Fat |
| 4.7 kcal/g | 3.8 kcal/g | 3.1 kcal/g | Energy |

MKO Myofibers Show Higher Levels of Respiration and Glucose Uptake than WT Myofibers:

To monitor the effect of MTCH2 KO on the metabolic state of myofibers, MTCH2 myofibers were isolated. In short, MTCH2 floxed male mice (MTCH2 f/f mice; 3-7 weeks old; pure C57b1/6 strain) were used for the isolation of satellite cells from the gastrocnemius skeletal muscle. To create MTCH2 KO myoblasts in vitro, myoblasts prepared from the MTCH2 f/f muscles were treated with purified recombinant Cre recombinase. To investigate the metabolic state of the MKO fibers, the respiration rate of f/f and f/f Cre cells was monitored utilizing the Seahorse Extracellular Flux (XF) Analyzer. The Seahorse XF analyzer detects real time changes in the Oxygen Consumption of intact cells [expressed as Oxygen Consumption Rate (OCR) and Spare Respiratory Capacity (SRC; maximal respiration)]. Maximal respiration is induced by Carbonyl cyanide-p-trifluoromethoxyphenylhydrazone, (FCCP), a protonophore that acts as a mitochondrial uncoupler to allow maximal respiration. OCR and SRC are expressed as units of pmoles/minute and all the assays were normalized to protein levels. Using this instrument the present inventors could compare the OCR and SRC of f/f and f/f Cre myofibers. The respiration of myofibers when cultured in the presence of glucose and pyruvate was examined. FIG. 7B shows that the basal OCR of the f/f Cre fibers was significantly higher than the basal OCR of f/f fibers. Furthermore, a larger difference was observed when FCCP was added (see maximal rates). Next the present inventors examined the respiration of myofibers when cultured in the presence of fatty acids using palmitate. The basal OCR was measured before adding palmitate, and the SRC was measured after palmitate addition. As expected, the basal OCR of the f/f Cre fibers was significantly higher than the basal OCR of the f/f fibers. Furthermore, a similar difference was detected in the SRC following palmitate addition (FIG. 7C). These studies suggest that MTCH2 is also involved in the regulation of oxidative phosphorylation driven by fatty acids. They next monitored glucose uptake in muscle fibers in vitro. Fully differentiated KO and f/f muscle fiber cells were incubated overnight in serum-free medium containing low glucose (5 mM). Next day, 2- [N-(7-nitrobenz-2-oxa-1,3-diazol-4-yl)amino]-2-deoxy-D-glucose (2-NBDG) was added to the cells for 30 minutes in the absence of glucose. The 2-NBDG uptake assay revealed that KO cells uptake significantly more glucose than f/f cells when incubated in low glucose (FIG. 7B).

Knocking Out MTCH2 Leads to a Significant Increase in Mitochondrial Size in Skeletal Muscle Fibers In Vivo

To determine whether knocking out MTCH2 in the muscle results in structural changes in the mitochondria electron microscope (EM) analyses were performed on the gastrocnemius skeletal muscle fibers. Interestingly, LKO gastrocnemius muscle fibers showed a significant increase in mitochondrial size (FIGS. 8A-B).

MKO Mice are Better at Endurance Exercise than WT Mice

To begin to assess the effect of MTCH2 KO on muscle biology the performance of WT and MKO mice was examined on a treadmill instrument (TSE Systems). The TSE Treadmill System is a fully computerized electronically controlled system to investigate exercise physiology in mice. The treadmill is driven by a servo controlled motor which provides precise operator-defined speed profiles. In the cases in which the mouse does not successfully cope with the defined speed profile and starts to get tired it reaches the end of the floor grids and received a weak electric shock. The number of shocks the mouse get reflects its motoric ability. An exhaustion profile (which contains acceleration of speed in each minute) was applied (FIG. 9A). The shocks served as an indication for the exhaustion, and the measurement is the distance that the mice achieved before it got exhausted (20 shocks per minute for continuous 5 minutes). The MKO mice actually run more distance before the exhaustion and therefore performed better (FIG. 9B). To understand the reason for the different performances, the present inventors monitored the respiration of the mice during the exercise period. The Treadmill System also contains a calorimetric configuration (CaloTreadmill) for concomitant metabolic measurements. This system can be connected to the metabolic cages LabMaster system (TSE-systems). The calorimetry system is an open-circuit system that determines O₂ consumption and CO₂ production. FIG. 9C shows that the MKO mice demonstrate higher oxygen consumption as compared to the WT mice.

Knocking Out MTCH2 Leads to a Significant Increase in Energy Expenditure

MTCH2^{F/F} MCK-Cre⁺ mice showed significance increase in energy expenditure. Energy expenditure is expressed as Heat production in absolute values (kcal/24 h) and statistically controlled for lean body weight, as illustrated in FIGS. 9D and 9E.

Knocking Out MTCH2 Leads to a Significant Increase in Cardiac Function

Echocardiographic analysis showed that similar heart size measurements: (9F) diameter (9G) systole and (9H) diastole left ventricular anterior wall (LVAW) and left ventricular posterior wall (LVPW) were found in both control and MTCH2^{F/F} MCK-Cre⁺ mice. However, MTCH2^{F/F} MCK-Cre⁺ mice showed increase in heart function measurements: (9I) ejection fraction (EF) (9J) stroke volume and (9K) fractional shortening (FS).

MTCH2 Early Muscle-Conditional Knockout Mice (MyKO) are Born Smaller Than WT Mice

To generate a MTCH2 early muscle-conditional knockout mouse, the MTCH2 f/f mice were bred with mice carrying a transgene for Cre-recombinase expressed under control of the Myf5 promoter, which is the earliest to be expressed among the myogenic regulatory factors (MRFs). Western blot analysis of several tissues prepared from the MTCH2 f/f and MTCH2 f/f myf5 mice demonstrated the absence of MTCH2 from skeletal muscle, while, interestingly, increased levels of MTCH2 in the heart, liver and brain tissues (FIG. 10). These results indicate specific deletion of MTCH2 in the skeletal muscle (in the MTCH2 f/f myf5 mice; MyKO mice for short), which seems to result in a compensation/upregulation of MTCH2 in other tissues.

After obtaining several litters from the MyKO mice, it was noticed that the MyKO pups are smaller than the MTCH2 f/f pups. FIG. 11A shows photographs of 2 MyKO mice on the left side and one WT mouse on the right side. To further follow this phenomenon, the pups were tattooed and their weight was monitored before and after weaning. FIG. 11B demonstrates that before weaning MyKO females have significantly less weight. This phenotype remained also after weaning (FIG. 11C). This phenomenon was also observed in male mice (FIGS. 11D and 11E).

### Example 3

### The Role of MTCH2 in the Brain and in the Testis

The brain is the centre of the nervous system and its function is to exert centralized control over the other organs of the body. Mitochondria are key organelles that allow efficient energy production to sustain neuronal membrane potential and firing activity. When mitochondrial functionality is compromised, changes in cellular energetics are observed and the whole-body energy balance is affected. Mitochondrial carrier homolog 2 (MTCH2) is an outer membrane mitochondrial protein, identified as a receptor-like protein for the pro-apoptotic BID protein. To determine the functional importance of MTCH2 in the brain, the present inventors generated a MTCH2 forebrain-conditional knockout mouse (BKO) using the CamKII-Cre deleter mice.

Results

Knocking out MTCH2 in mice resulted in embryonic lethality; therefore, to explore the role of MTCH2 in the brain, MTCH2^{f/f} mice were mated with mice carrying a transgene for Cre-recombinase expressed under control of the alpha subunit of the calcium/calmodulin-dependent protein kinase II (CamKIIa) promoter. Initially, CamKIIa cre mice were crossed with ROSA26 reporter mice to monitor the tissue/cellular expression pattern of the cre transgene in these mice (and thus where to expect MTCH2 KO) (FIGS. 12A-B).

BKO Mice Gain Less Weight on Regular Chow Diet

BKO mice and their wild-type littermates were monitored every week, at the same hour, for 3 months. In the first three weeks after birth there were no differences. However, BKO female mice started to show less weight gain from the 6th week of age (FIG. 13), and the males starting from the 10th week of age (data not shown).

BKO Females are More Active During the Night Cycle and Consume More Food

TSE Labmaster/ Phenomaster Platform (TSE System) was used for indirect calorimetry and activity measurement on 11-week old BKO and MTCH2f/f females (8 from each genotype) of matched body weight. Food intake, respiratory exchanges and energy expenditure were measured when mice were fed ad libitum (for 3 days) and also during the re-feeding response after starvation. BKO mice are hyperactive during the night cycle and, probably as a result of increased locomotor activity, they display higher oxygen consumption as well as increased CO2 and heat production (FIGS. 14A-D and FIGS. 16A-B).

The cumulative food intake during the 3-day period also tends to be higher (FIG. 15B; p=0,06), meaning that the BKO mice consume more food and this can be possibly due to their increased activity and/or the lower leptin levels detected in their serum (FIGS. 16A-B).

Mice during fasting and re-feeding were also examined the. BKO females showed higher locomotor activity during an overnight fast; suggesting increased foraging behaviour (data not shown). Moreover, they showed significantly higher rebound food intake after the O.N. fasting (FIG. 14B; p=0,03).

Behavioral characterization of BKO females: BKO mice, when housed with a running wheel, cover more distance then wild type littermates (FIG. 16A); although they seem to have impaired motor planning and coordination as they perform worse in the rotarod test (FIG. 16C). In correlation with this last finding, BKO mice display impaired learning and spatial memory disability when challenged in the Morris Water Maze task (FIGS. 16D and E).

FIG. 16F illustrates that administration of Ritalin represses the hyperactivity in BKO mice.

It was shown that BKO females are more active than the wild-type littermates (MTCH2^{F/F} vs BKO injected with saline; p=0.034; 2way ANOVA- Multiple comparison). The same mice receive 2 mg/kg intraperitoneal injection of ritalin (Novartis), notably the BKO treated with RITALIN behave like the control group (BKO saline vs BKO 2 mg/Kg Ritalin; p=0.03; 2way ANOVA- Multiple comparison).

BKO males are infertile due to cre expression in the testis: Surprisingly, abundant Cre-recombinase expression was detected in the testis, whereas in all the other organs, as predicted, the Cre is not expressed. Cre recombinase leakage in the male reproductive system results in a 90% reduction in MTCH2 mRNA levels. When F3 BKO males were mated with MTCH2f/f females no litters were born. Thus, the present inventors decided to further investigate this phenomenon and set up a fertility experiment: two-months-old males were mated with 2 C57BL6J females for a period of 4 months. The total number of pups per male was recorded at birth. In the breeding cage with the MTCH2f/f male, 71 pups were counted (FIG. 17A). On the other hand, only in one of the 5 cages with the BKO males, 4 pups were born. The pups were found dead on the same day they were born, presumably killed by the parents; for this reason it was impossible to identify any defects in the newborns. To assess spermatozoa motility, mature spermatozoa released from ductus deferens of 3 BKO and 3 MTCH2f/f mice were collected. Sperm numbers and motility were evaluated after incubation at 37° C. for 30 min in M199 medium (Invitrogen). BKO mice had severe hypospermia compared to WT mice (data not shown) and the overall motility is dramatically reduced (FIG. 17B).

Histological analysis of BKO testis show that there is a severe degeneration of the seminiferous tubules (ST): the number of spermatozoa is severely reduced as well as the spermatogenic cells lining the ST (FIGS. 18A-I). In addition, macrophages and increased amount of eosinophilic debris and scattered apoptotic/necrotic cells were found in the BKO testis. However, the number of Leydig cells is comparable to the MTCH2f/f.

The epididymis is composed by tubules that collect a large number of mature spermatozoa. In the BKO mice, there is a profound reduction in the number of mature spermatozoa. In the tail of the epididymis, the few mature spermatozoa present are admixed with abundant eosinophilic debris and macrophages (FIGS. 19A-H). In the head, spermatozoa are very rare; some tubules contain eosinophilic material and a low number of macrophages (FIGS. 19A-H).

### Example 4

### The role of MTCH2 in the Liver

Liver specific deletion of MTCH2 results in accelerated respiration on succinate, a complex II substrate: To explore whether MTCH2 is involved in OXPHOS regulation, the respiration rate of LKO and WT control purified liver mitochondria was monitored using a Clarke-type oxygen electrode. Using these mitochondria the present inventors measured respiration induced by substrates for individual electron transport chain complexes in the presence/absence of appropriate inhibitors. Succinate, a substrate for respiration via complex II, gave the most profound effect. In the presence of succinate the LKO mitochondria showed a two-fold increase in state 3 respiration rate compared to the WT control and also state 4 respiration rate was significantly increased (FIG. 20). “State 3” is the active respiring state that starts upon ADP addition to mitochondria in the presence of excess substrate, while the slower rate, after all the ADP has been phosphorylated to form ATP, is referred to as “state 4”. State 4 is also achieved by the addition of oligomycin that prevents state 3 respiration completely by inhibition of the ATP synthase (inhibitor of OXPHOS), and allowing only state 4 respiration (i.e., it has no direct effect on electron transport or the chemiosmotic gradient). Thus, MTCH2 deficiency alters both the coupled respiration state (state 3) which consists of both electron transport chain (ETC) and ATP synthesis activities, and the leaky or uncoupled respiration state (state 4) which is composed only from the ETC activity.

LKO isolated hepatocytes show an elevated rate of 13-oxidation: Using primary cultured hepatocytes the present inventors addressed whether MTCH2 modulates hepatic lipid catabolism, also known as FA (3-oxidation, a process that takes place at the mitochondria [9,10-³H]Myristate and [9,10-³H]Palmitate are commonly used in ³H20 release assays for detecting medium- and long-chain FA oxidation defects by fibroblast monolayers from patients with FA oxidation disorders. FA (3-oxidation was measured in primary hepatocytes isolated from LKO and WT livers by assessing the ability of the cells to convert tritium-radiolabled long chain FAs: Palmitate (C-16) and Myristate (C-14) into radiolabled-water (see FIG. 21A for the experimental description). MTCH2/MIMP deficient primary hepatocytes exhibited significantly higher rates of (3-oxidation compared to the WT hepatocytes when using both Myristate and Palmitate as substrates (FIG. 21B).

LKO livers have elevated levels of ATP: The elevation in OXPHOS and FA β-oxidation in the LKO mitochondria/hepatocytes may arise due to a compensatory mechanism(s). One hypothesis is that the MTCH2 deficient mitochondria have a defect in ATP production. Thus, the accelerated oxygen consumption rate could be explained by the need of the cell to overcome this problem. If this theory were true it may be anticipated that the ATP content in the livers of MTCH2 mice would be lower or equal to that of WT controls. In contrast to this hypothesis, but in correlation with all the other mitochondrial activities that were elevated in the LKO mice, also the ATP content was higher in liver homogenates of LKO mice compared to WT littermate controls (FIG. 22). A possible explanation for these results is that LKO mitochondria preferentially use FAs over glucose to generate ATP. Since, catabolism of one molecule of palmitate (C16) yields 129 ATP molecules whereas, catabolism of one glucose molecule produces 38 ATP molecules, preferentially using FAs will result in higher levels of ATP.

LKO Mice Gain Less Weight than WT Mice on High Fat Diet

LKO mice and their corresponding WT control littermates were challenged with high fat diet (HFD). LKO (n=10) and WT (n=7) mice were fed on a HFD (45% fat energy) for 27-32 weeks, and a parallel cohort of LKO (n=5) and WT (n=4) mice was fed on normal diet (ND; 10% fat energy) for 27 weeks. WT mice fed on HFD for 15 weeks gained significantly more weight compared to WT mice fed on ND (FIGS. 23A and 23B). In contrast, LKO mice did not gain significantly more weight when fed on HFD compared to ND (FIGS. 23A and 23B; of note, no differences in food intake between the two groups was found). To attempt to define the reason(s) for these differences a variety of biochemical parameters in the serum of the mice (before and 14 weeks after the start of the diet) were analyzed that included glucose, total cholesterol, HDL cholesterol, triglycerides, total bilirubin, blood urea and the activity of liver enzymes (AST, ALT, LDH). The only parameter that showed a significant difference between the mice was triglycerides (TG). The serum TG levels were significantly higher in the WT mice compared to the LKO mice fed on HFD for 14 weeks (FIG. 23C). Interestingly, there was also a significant (but smaller) difference between the WT and LKO mice at the beginning of the experiment (FIG. 23C, 0 weeks) suggesting that the absence of MTCH2 is affecting the serum TG levels on normal diet and the HFD enhances this affect.

MTCH2 deletion in the liver has profound effects on hepatic fatty acid levels: The results described above suggest that MTCH2 plays a role in fatty acid (FA) metabolism. To assess whether MTCH2 deletion has an effect on lipid levels that are related to FA metabolism in the liver the present inventors performed global analysis of lipids from WT and LKO liver samples using multidimensional mass spectrometry-based shotgun lipidomics (MDMS-SL). MDMS-SL is a matured technology for the automated identification and quantification of individual lipid molecular species. The initial results show a profound difference in the levels of several lipid classes between the WT and LKO liver samples (FIG. 24). One of the interesting differences is in the levels of acyl-carnitine, which is an important indicator of mitochondrial activity. The fact that the acyl-carnitine levels are higher in the LKO livers suggests that MTCH2 deletion increases the mitochondrial uptake of FA, which may lead to an increase in FA oxidation.

### Example 5

The role of MTCH2 in Regulating Embryonic Stem Cells During Embryogenesis Abstract

MTCH2^{-/-} mESC Possess Growth and Metabolic Defects Complete knockout of MTCH2 in mice results in embryonic lethality at E7.5. In order to further understand the reasons behind this effect, the present inventors generated stable clones of MTCH2^{-/-} mESC from E3.5 MTCH2^{F/F} blastocysts treated in vitro with recombinant Cre-recombinase. Initially, they assessed the growth properties of these cells and found that the MTCH2^{-/-} mESC grew significantly slower (FIG. 25A). To assess whether this growth impairment was related to the pluripotency of the cells, they monitored the expression of the core pulripotency markers OCT-4, SOX2, Klf-4 and Nanog, and found no differences (not shown). The embryoid bodies (EB's) formation assay was used to test the cells wherein no significant differences in the expression of gene markers for each of the three different linages were found. To address the growth and pluripotency properties in vivo, a teratoma assay was performed. Mice were injected with either MTCH2^{F/F} or MTCH2^{-/-} stable mESC clones, and teratoma/tumor generation was monitored. Three weeks post injection the tumors were removed, weighed, followed by histology analysis of each tumor separately. The tumors obtained from the MTCH2^{-/-} mESC were much smaller in size and less vascularized when compared with tumors obtained from the MTCH2^{F/F} cells (FIG. 25B). Histology analysis revealed that the MTCH2^{-/-} cells were able to differentiate properly to all three germ layers (FIG. 25C). These results together imply that MTCH2 knockout results in an isolated growth defect without perturbing mESC differentiation and pluripotency. Based on the proliferation impairment described above, the present inventors decided to explore the metabolic activity of the MTCH2^{-/-} mESC. Initially they determined the levels of lactate in the medium of the cells, and were surprised to find that they were significantly higher in the MTCH2^{-/-} mESC (FIG. 25D), although these cells are less proliferative. Surprisingly, these results seem to be the opposite from the results we obtained in MTCH2^{F/F} Vavl-cre⁺ HSPCs that show lower levels of lactate. To further explore this issue in mESCs dynamic flux of metabolites using gas chromatography mass-spectroscopy (GC-MS) analysis with uniformly labeled ¹³C-Glucose and ¹³C-Glutamine was performed, to determine the rate of pyruvate oxidation and glutaminolysis, respectively (FIG. 25E). Consistent with the initial results, MTCH2^{-/-} mESC showed higher lactate production, as concluded from the higher levels of labeled M+3 lactate arising from ¹³C-labeled glucose (FIG. 25F). However, no differences in the levels of alanine production nor in the levels of the intermediate TCA cycle products (not shown) were detected. These results suggest that less pyruvate is entering the TCA cycle in the MTCH2^{-/-} mESC and more is converted to lactate and is secreted to the medium. Interestingly, MTCH2^{-/-} mESC showed a higher rate of glutaminolysis, as concluded from the higher levels of all the TCA cycle metabolites arising from ¹³C-labeled glutamine (FIG. 25G). These results suggest that the MTCH2^{-/-} mESC hyper-utilize glutamine to replenish the TCA cycle, perhaps to compensate for the lower levels of pyruvate oxidation. Together with the high OXPHOS detected in HSPCs, these results could suggest mixed metabolic signaling to stem cells which disturbs their ability to either proliferate (mESC) or to maintain quiescent (HSC).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating a mitochondrial related disease or condition in a subject in need thereof comprising administering to the subject a therapeutically effective amount of an agent which regulates an amount and/or activity of mitochondrial carrier homologue 2 (MTCH2) or an agonist thereof, thereby treating the mitochondrial related disease or condition.

2. (canceled)

3. The method of claim 1, wherein said agonist comprises BH3-interacting domain death agonist (BID).

4. The method or of claim 1, wherein said regulates is down-regulates.

5. The method of claim 4, wherein said disease or condition is a fat-related disease, radiation injury, reduced exercise endurance or a cardiac disease.

6-7. (canceled)

8. The method of claim 5, wherein said fat-related disease is selected from the group consisting of diabetes, metabolic syndrome and obesity.

9. (canceled)

10. The method of claim 4, wherein said agent is an oligonucleotide directed to an endogenous nucleic acid sequence expressing said MTCH2 or said agonist thereof.

11. The method of claim 1, wherein said regulates is up-regulates.

12. The method of claim 11, wherein said disease or condition is selected from the group consisting of retarded growth, hyperactivity, learning and memory disability, a hematological cancer and male infertility.

13. A method of increasing mobilization of hematopoietic precursors from the bone marrow to the peripheral blood in a subject in need thereof, the method comprising
administering to the subject an agent which downregulates an activity and/or expression of a component participating in the mitochondrial apoptotic pathway.

14-16. (canceled)

17. The method of claim 13, wherein said subject is a donor subject.

18. The method of claim 13, wherein said subject is a recipient subject or a recipient subject in need of an organ transplantation.

19-22. (canceled)

23. The method of claim 13, wherein said subject has an immune deficiency.

24-27. (canceled)

28. The method of claim 13, wherein said at least one component participating in the mitochondrial-related apoptotic pathway is selected from the group consisting of mitochondrial carrier homologue 2 (MTCH2), BID, Caspase-8, Bax and Bak.

29. The method of claim 13, wherein said at least one component participating in the mitochondrial-related apoptotic pathway is MTCH2.

30. A method of propagating stem cells comprising culturing the stem cells in a culture medium comprising an agent that upregulates an activity and/or expression of a component participating in the mitochondrial apoptotic pathway under conditions that allow propagation of the stem cells, but do not allow differentiation of the stem cells, thereby propagating the stem cells.

31-32. (canceled)

33. The method of claim 30, wherein said stem cells comprise pluripotent stem cells.

34. (canceled)

35. The method of claim 30, wherein said stem cells comprise hemapoietic stem cells.

36. The method of claim 33, wherein said at least one component participating in the mitochondrial-related apoptotic pathway is selected from the group consisting of mitochondrial carrier homologue 2 (MTCH2), BID, Caspase-8, Bax and Bak.

37. The method of claim 30, wherein said at least one component participating in the mitochondrial-related apoptotic pathway is MTCH2.

38-39. (canceled)

40. The method of claim 13 comprising harvesting said hematopoietic precursors from the peripheral blood.
